(19) United States
(12) Patent Application Publication
(10) Pub. No.: US 2024/0059685 A1
(43) Pub. Date: Feb. 22, 2024

(54) PIEZO1 AGONISTS FOR THE PROMOTION OF BONE FORMATION

(71) Applicant: BIOVENTURES, LLC, Little Rock, AR (US)

(72) Inventors: Hong-yu Li, Little Rock, AR (US); Jinhu Xiong, Little Rock, AR (US); Wei Yan, Little Rock, AR (US); Charles O'Brien, Little Rock, AR (US); Maria Schuller De Almeida, Little Rock, AR (US)

(21) Appl. No.: 17/766,498

(22) PCT Filed: Oct. 5, 2020
(86) PCT No.: PCT/US2020/054279

§ 371 Date: Apr. 4, 2022

Related U.S. Application Data
(60) Provisional application No. 62/910,773, filed on Oct. 4, 2019.

(51) Int. Cl. C07D 417/04; A61P 19/08

(52) U.S. Cl. CPC C07D 417/04; A61P 19/08

(57) ABSTRACT

Disclosed herein are Piezo1 agonists. Also disclosed herein are methods of stimulating tissue anabolism in a subject comprising administering an effective amount of a Piezo1 agonist and methods for chemically mimicking mechanical stimulation of a cell expressing Piezo1 comprising contacting a cell expressing Piezo1 with an effective amount of a Piezo1 agonist.

### CROSS-REFERENCE TO RELATED APPLICATIONS

This application claims priority to U.S. Provisional Application No. 62/910,773, filed Oct. 4, 2019, the contents of which are incorporated by reference in its entirety.

### REFERENCE TO SEQUENCE LISTING

This application is being filed electronically via EFS-Web and includes an electronically submitted Sequence Listing in .txt format. The .txt file contains a sequence listing entitled “169852_00074_ST25.txt” created on Sep. 28, 2020 and is 3.05 KB in size. The Sequence Listing contained in this .txt file is part of the specification and is hereby incorporated by reference herein in its entirety.

### BACKGROUND

Mechanical signals play critical roles in bone growth and homeostasis (Turner et al., 2009, Ozcivici et al., 2010). Mechanical stimuli increase bone mass by stimulating the activity and production of bone forming osteoblasts (Meakin et al., 2014, Klein-Nulend et al., 2012). In contrast, loss of mechanical signals decreases bone mass by reducing bone formation and stimulating production of bone resorbing osteoclasts (Kondo et al., 2005, Nakamura et al., 2013, Xiong et al., 2011). Osteocytes, which are cells buried in the bone matrix and derived from osteoblasts, are able to sense changes in mechanical load and orchestrate bone resorption and formation (Bonewald, 2011, Klein-Nulend et al., 2013). However, the molecular mechanisms by which osteocytes sense changes in mechanical loads remain unclear.

A variety of cell surface proteins and structures, including integrins, focal adhesions, and primary cilia, have been proposed to sense mechanical signals in bone cells (Litzenberger et al., 2010, Nguyen and Jacobs, 2013, Rubin et al., 2006). In addition, several lines of evidence suggest that ion channels are involved in the sensing of mechanical signals by osteocytes (Hung et al., 1995, Lu et al., 2012, Lewis et al., 2017, Li et al., 2002). These differing theories suggesting numerous different cell surface proteins, structures, or ion channels highlight the lack of definitive understanding of the molecular mechanisms for promoting bone formation in response to mechanical loading. Without such understanding, there is no insight into which cell surface proteins, structures, or ion channels to target or compounds capable of doing so to mimic mechanical loading for the promotion of bone formation and health.

### BRIEF SUMMARY OF THE INVENTION

Disclosed herein are Piezo1 agonists. Also disclosed herein are methods of stimulating tissue anabolism in a subject comprising administering an effective amount of a Piezo1 agonist and methods for chemically mimicking mechanical stimulation of a cell expressing Piezo1 comprising contacting a cell expressing Piezo1 with an effective amount of a Piezo1 agonist.

In one aspect of the invention Piezo1 agonists are disclosed. The Piezo1 agonists may be a compound having formula (I) or a pharmaceutically acceptable salt thereof.

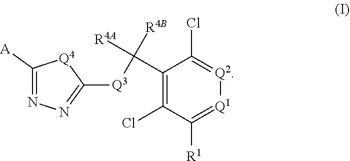

In some embodiments, Q¹ and Q² are CR² and CR³, respectively, and each of R¹, R², and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl. In particular embodiments, R¹, R², and R³ are not each hydrogen. R^{4A} and R^{4B} may be independently selected from hydrogen or a C₁-C₆ alkyl or R^{4A} and R^{4B} are together selected from an oxo group. Q³ may be selected from S, O, or NH. Q⁴ may be selected from S, O, or NR⁷, wherein R⁷ is selected from hydrogen, a C₁-C₆ alkyl, or an aryl. A is a substituted or unsubstituted heteroaryl having at least 1 N in the heteroaryl ring.

In some embodiments, Q¹ and Q² are CR² and N, respectively, and each of R¹ and R² are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl.

In some embodiments, Q¹ and Q² are N and CR³, respectively, and each of R¹ and R³, are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl.

Another aspect of the invention comprises a method for stimulating tissue anabolism. The method may comprise administering to the subject an effective amount of a Piezo1 agonist or a pharmaceutical composition comprising the effective amount of the Piezo1 agonist, wherein the tissue comprises cells expressing Piezo1.

Another aspect of the invention comprises a method for chemically mimicking mechanical stimulation of a cell expressing Piezo1. The method may comprise contacting the cell expressing Piezo1 with an effective amount of a Piezo1 agonist.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1F demonstrate that Piezo1 mediates mechanotransduction in an osteocyte cell line. (FIG. 1A) mRNA levels of calcium channels regulated by fluid shear stress in MLO-Y4 cells determined by RNA-seq (here and throughout, values are the mean±s.d.). (FIG. 1B) qPCR of Piezo1 and Piezo2 mRNA in MLO-Y4 cells cultured under static or fluid shear stress conditions for 2 hours. *p<0.05 versus static, using Student's t-test. (FIG. 1C) Piezo1 and Piezo2 mRNA levels in cortical bone of 3-month-old wildtype C57BL/6J mice. (FIG. 1D) Intracellular calcium concentration measured in control or Piezo1 knock-down MLO-Y4 cells before and after the start of fluid flow. Arrow indicates the time when fluid flow starts. (FIG. 1E) qPCR of Piezo1, Ptgs2, and Tnfrsf11b in control or Piezo1 knock-down MLO-Y4 cells cultured under static or fluid shear stress conditions for 2 hours. n=3 per group. (FIG. 1F) qPCR of Piezo1, Ptgs2, and Tnfrsf11b in control or Piezo1 overexpressed MLO-Y4 cells cultured under static or fluid shear stress conditions for 2 hours. n=3 per group. *p<0.05 with the comparisons indicated by the brackets using 2-way ANOVA. Left bar of each paired set indicates the static condition and right bar indicates fluid shear stress.

FIGS. 2A-2M demonstrate that loss of Piezo1 in osteoblasts and osteocytes decreases bone formation and bone mass. (FIG. 2A) qPCR of loxP-flanked Piezo1 genomic DNA isolated from tibial cortical bone of Dmp1-Cre; Piezo1^{f/f} (n=6) and Piezo1^{f/f} (n=6) littermates. *p<0.05 using Student's t-test. (FIG. 2B) Serial BMD of female Dmp1-Cre; Piezo1^{f/f} mice and their littermate controls at 5, 8, and 12 weeks of age. *p<0.05 using 2-way ANOVA at a given age. (FIG. 2C) X-ray images of tibia from 12-week-old Dmp1-Cre; Piezo1^{f/f} and Piezo1^{f/f} littermate. Arrowhead indicates the location of fracture. Representative μCT images (scale bar, 0.1 mm) (FIG. 2D) and cortical thickness, periosteal circumference, and endocortical circumference analysis (FIG. 2E) of the femoral diaphysis in Dmp1-Cre; Piezo1^{f/f} (n=9) and Piezo1^{f/f} (n=9) littermates. (FIG. 2F) Cortical thickness measured in the 4th lumbar vertebra of 12-week-old female Dmp1-Cre; Piezo1^{f/f} (n=9) and Piezo1^{f/f} (n=9) littermates. (FIG. 2G) Bone volume per tissue volume (BV/TV) measured in the femur and the L4 vertebra of 12-week-old female Dmp1-Cre; Piezo1^{f/f} (n=9) and Piezo1^{f/f} (n=7) mice. (FIG. 2H) Representative μCT images of the distal femur. Scale bar, 1 mm. (FIG. 2I) Stiffness, ultimate force, Young's modulus, and ultimate stress measured in the femurs of Dmp1-Cre; Piezo1^{f/f} (n=9) and Piezo1^{f/f} (n=9) littermates. (FIG. 2J) Tissue mineral density measured in cortical bone in femoral diaphysis of Dmp1-Cre; Piezo1^{f/f} (n=9) and Piezo1^{f/f} (n=9) littermates. (FIG. 2K) Representative histological cross sections (left, yellow dotted line indicates periosteal surface and white dotted line indicates endocortical surface; scale bar=100 μm ) and quantification of mineralizing surface in periosteal and endocortical surface (right) at the femoral diaphysis of 5-week-old female Dmp1-Cre; Piezo1^{f/f} (n=7) and Piezo1^{f/f} (n=5) littermates. (FIG. 2L) Mineralizing surface per bone surface (MS/BS), mineral apposition rate (MAR), and bone formation rate per bone surface (BFR/BS). (FIG. 2M) Osteoblast number (N.Ob/B.Pm), and osteoclast number (N.Oc/B.Pm) measured in cancellous bone of lumbar vertebra 1-3 from 12-week-old female Dmp1-Cre; Piezo1^{f/f} (n=5) and Piezo1^{f/f} (n=5) littermates. *p<0.05 using Student's t-test. Left bar of each paired set indicates Piezo1^{f/f} and right bar indicates Dmp1-Cre; Piezo1^{f/f}.

FIGS. 3A-3D demonstrate that loss of Piezo1 in osteoblasts and osteocytes blunts the skeletal response to mechanical loads. (FIG. 3A) Schematic illustration of anabolic loading on mouse tibia. (FIG. 3B) Cortical thickness (Ct.Th) in the tibial shaft of 4-month-old loaded or control Dmp1-Cre; Piezo1^{f/f} (n=5) and Piezo1^{f/f} (n=7) littermates. (FIG. 3C) Mineralizing surface (MS/BS), mineral apposition rate (MAR), and bone formation rate (BFR/BS) in periosteal surface of the tibia of 4-month-old female Dmp1-Cre; Piezo1^{f/f} (n=5) and Piezo1^{f/f} (n=7) littermates. (FIG. 3D) Representative histological cross section images of the tibial shaft of 4-month-old female Dmp1-Cre; Piezo1^{f/f} and Piezo1^{f/f} littermates. Scale bar, 100 μm. *p<0.05 with the comparisons indicated by the brackets using 2-way ANOVA. Left bar of each paired set indicates control and right bar indicates loaded.

FIGS. 4A-4G demonstrate that Piezo1 controls Wnt1 expression via YAP1 and TAZ. (FIG. 4A) qPCR of Wnt1 mRNA in tibial cortical bone of 5-week-old female Piezo1^{f/f} (n=6) and Dmp1-Cre; Piezo1^{f/f} mice (n=6). *p<0.05 using Student's t-test. (FIG. 4B) Relative mRNA levels of Wnt1, Sost, Tnfsf11 (RANKL), and Tnfrsf11b (OPG) in tibia cortical bone of 12-week-old female Piezo1^{f/f} (n=9) and Dmp1-Cre; Piezo1^{f/f} (n=9) mice. *p<0.05 using Student's t-test. (FIG. 4C) Wnt1 and Cyr61 mRNA levels in control or Piezo1 knock-down MLO-Y4 cells cultured under static or fluid shear stress conditions. *p<0.05 with the comparisons indicated by the brackets using 2-way ANOVA. (FIG. 4D) Ptgs2, Wnt1, and Cyr61 mRNA levels in control or Yap1/Taz knock-down MLO-Y4 cells cultured under static or fluid shear stress conditions. *p<0.05 with the comparisons indicated by the brackets using 2-way ANOVA. (FIG. 4E) YAP1 immunofluorescence in control or Piezo1 knock-down MLO-Y4 cells cultured under static or fluid shear stress conditions. Scale bar, 100 μm. (FIG. 4F) Quantification of mean fluorescence intensity in nucleus versus cytoplasm in the cells described in (FIG. 4E). (FIG. 4G) Wnt1 and Cyr61 mRNA levels measured in tibia of female Dmp1-Cre; Piezo1^{f/f} (n=8) and Piezo1^{f/f} (n=7) mice loaded with one bout of compressive loading. Mice were harvested 5 hours after loading. *p<0.05 with the comparisons indicated by the brackets using 2-way ANOVA. FIGS. 4A-4B: Left bar of each paired set indicates Piezo1^{f/f} and right bar indicates Dmp1-Cre; Piezo1^{f/f}. FIGS. 4C-4D, and 4F: Left bar of each paired set indicates static and right bar indicates fluid flow. FIG. 4G: Left bar of each paired set indicates control and right bar indicates loaded.

FIGS. 5A-5I demonstrate that activation of Piezo1 mimics the effects of mechanical stimulation on osteocytes. (FIG. 5A) Intracellular calcium concentration measured in control or Piezo1 knock-down MLO-Y4 cells immediately after the treatment of DMSO or 10 μM Yoda1. (FIG. 5B) qPCR of Ptgs2, Wnt1, and Tnfrsf11b in control or Piezo1 knock-down MLO-Y4 cells treated with DMSO or 10 μM Yoda1 for 2 hours. n=3 per group. *p<0.05 versus vehicle treated controls of the same genotype by 2-way ANOVA. (FIG. 5C) qPCR of Ptgs2, Wnt1, and Tnfrsf11b in control or Yap1/Taz knock-down MLO-Y4 cells treated with DMSO or 10 μM Yoda1 for 2 hours. n=3 per group. *p<0.05 versus vehicle treated controls of the same genotype by 2-way ANOVA. (FIG. 5D) qPCR of Ptgs2, Wnt1, Tnfrsf11b, Cyr61, and Sost in ex vivo cultured femoral cortical bone from 5-week-old mice treated with DMSO or 10 μM Yoda1 for 4 hours. n=3 per group. (FIG. 5E) qPCR of Wnt1 in tibia of C57BL/6J mice treated with Vehicle or Yoda1 for 4 hours. n=12 per group. (FIG. 5F) Schedule of in vivo Yoda1 administration. Cortical thickness and cancellous BV/TV in distal femur (FIG. 5G) and the 4^{th} lumbar (FIG. 5H) of 4-month-old vehicle or Yoda1 treated female C57BL/6J mice (n=12 per group). (FIG. 5I) Circulating osteocalcin levels in the serum of 4-month-old vehicle or Yoda1 treated female C57BL/6J mice (n=12 per group). *p<0.05 versus vehicle treated controls by Student's t-test. Left bar of each paired set indicates vehicle and right bar indicates Yoda1.

FIGS. 6A-6B show a sequencing analysis of mRNA isolated from MLO-Y4 cells cultured under static or fluid shear stress conditions. (FIG. 6A) PCA analysis of RNAseq. (FIG. 6B) Volcano plot of differentially expressed transcripts in MLO-Y4 cells cultured under fluid shear stress (FF) versus static (ST) conditions.

FIGS. 7A-7B show the sequencing analysis of mRNA isolated from MLO-Y4 cells cultured under static or fluid shear stress conditions. (FIG. 7A) GO-enrichment analysis of genes isolated from MLO-Y4 cells cultured under fluid shear stress (FF) and static (ST) conditions. (FIG. 7B) Calcium channels expressed in MLO-Y4 cells.

FIGS. 8A-AJ show that loss of Piezo1 in osteoblasts and osteocytes decreases bone mass. (FIG. 8A) Body weight of female and male Dmp1-Cre; Piezo1^{f/f} (n=9 f, 8 m) and Piezo1^{f/f} (n=9 f, 9 m) mice at 12 weeks of age. (FIG. 8B) Femoral, spinal, and total body bone mineral density of male mice of indicated genotypes at 5, 8, and 12 weeks of age. (FIG. 8C) Femoral cortical thickness of 12-week-old male Dmp1-Cre; Piezo1^{f/f} (n=8) and Piezo1^{f/f} mice (n=9). (FIG. 8D) Total cross sectional area, cortical bone area, medullary area in the midshaft of femur in 12-week-old female Dmp1-Cre; Piezo1^{f/f} (n=5) and Piezo1^{f/f} (n=5) mice. (FIG. 8E) Femur length of 12-week-old female and male Dmp1-Cre; Piezo1^{f/f} (n=9 f, 8 m) and Piezo1^{f/f} (n=9 f, 9 m) mice. *p<0.05 using 2-way ANOVA. (FIG. 8F) Vertebral cortical thickness of 12-week-old male Dmp1-Cre; Piezo1^{f/f} (n=8) and Piezo1^{f/f} (n=9) mice. Trabecular number (Tb. N), trabecular thickness (Tb. Th), and trabecular separation (Tb. Sp) measured in the femur (FIG. 8G) and the L4 vertebra (FIG. 8H) of 12-week-old female Dmp1-Cre; Piezo1^{f/f} (n=9) and Piezo1^{f/f} (n=9) mice. Bone volume per tissue volume (BV/TV), trabecular number, trabecular thickness, and trabecular separation measured in the femur (FIG. 8I) and the L4 vertebra (FIG. 8J) of 12-week-old male Dmp1-Cre; Piezo1^{f/f} (n=8) and Piezo1^{f/f} (n=9) mice. *p<0.05 using Student's t-test. Left bar of each paired set indicates Piezo1^{f/f} and right bar indicates Dmp1-Cre; Piezo1^{f/f}.

FIGS. 9A-9F demonstrate that deletion of Piezo1 in osteoblasts and osteocytes decreases cortical bone. (FIG. 9A) Representative images of cross sections of femoral diaphysis from 5-week-old Dmp1-Cre; Piezo1^{f/f} and Piezo1^{f/f} mice. Empty lacunae (FIG. 9B) and osteocyte number (FIG. 9C) measured in the longitudinal section of L1-3 vertebrae in 12-week-old female Dmp1-Cre; Piezo1^{f/f} (n=5) and Piezo1^{f/f} (n=5) mice. (FIG. 9D) Representative histological images of osteocytes in vertebral cortical and cancellous bone of 12-week-old female Dmp1-Cre; Piezo1^{f/f} and Piezo1^{f/f} mice. (FIG. 9E) Caspase 3 activity measured in Piezo1 knock-down MLO-Y4 and control cells. (FIG. 9F) Alizarin Red staining of bone marrow stromal cells isolated from 5-week-old female Dmp1-Cre; Piezo1^{f/f} (n=3) and Piezo1^{f/f} (n=3) mice and cultured for 21 days in osteoblast differentiation medium (n=3 wells/group). Left bar of each paired set indicates Piezo1^{f/f} and right bar indicates Dmp1-Cre; Piezo1^{f/f}.

FIGS. 10A-10D show that deletion of Piezo1 from Dmp1-Cre-targeted cells does not affect muscle mass. (FIG. 10A) Abundance of Piezo1 genomic DNA measured by qRT-PCR in gastrocnemius of Dmp1-Cre; Piezo1^{f/f} and Piezo1^{f/f} mice. (FIG. 10B) Piezo1 mRNA in tibia and gastrocnemius muscle in male wild type mice (n=5). Lean body weight (FIG. 10C) and gastrocnemius muscle mass (FIG. 10D) measured in 12-week-old Dmp1-Cre; Piezo1^{f/f} (n=9 f, 8 m) and Piezo1^{f/f} (n=9 f, 9 m) mice. *p<0.05 using Student's t-test. Left bar of each paired set indicates Piezo1^{f/f} and right bar indicates Dmp1-Cre; Piezo1^{f/f}.

FIGS. 11A-11C demonstrates that loss of YAP1 and TAZ in osteoblasts and osteocytes decreases cortical bone. (FIG. 11A) Cortical thickness (left), periosteal circumference (middle), and endocortical circumference (right) at the femoral diaphysis of Dmp1-Cre; Yap1^{f/f}, Taz^{f/f} (n=10) and Yap1^{f/f}, Taz^{f/f} (n=12) littermates. Left bar of each paired set indicates Yap1^{f/f}Taz^{f/f} and right bar indicates Dmp1-Cre; Yap1^{f/f}, Taz^{f/f}. (FIG. 11B) mRNA of Piezo1 in control and Piezo1 knock-down MLO-Y4 cells. Left bar of each paired set indicates sh-luc and right bar indicates sh-Piezo1. (FIG. 11C) mRNA levels of Yap1 and Taz in control and Yap1/Taz knock-down MLO-Y4 cells. Left bar of each paired set indicates sh-luc and right bar indicates sh-Yap1/Taz. *p<0.05 using Student's t-test.

FIG. 12 demonstrates that deletion of Piezo1 in osteoblastic cells blunted their response to fluid flow. qRT-PCR of Piezo1, Ptgs2, Wnt1 , and Cyr61 in control (Cas9) and Piezo1 knockout (Cas9+Piezo1 sgRNAs) UAMS-32 cells cultured under static and fluid flow conditions. *p<0.05 using 2-way ANOVA. Osteoblastic UAMS-32 cells were transfected with Cas9 or Cas9 with two sgRNAs targeting introns 3 and 4 of the Piezo1 gene. Single Cells were then flow-sorted into 96-well plates for screening. Cells with homologous deletion of the exon 4 of Piezo1 were pooled together for analysis. Left bar of each paired set indicates static and right bar indicates fluid flow.

FIG. 13A shows Body weight of C57BL/6J mice before and after 2 weeks of vehicle or Yoda1 administration (n=12 mice per group).

FIG. 13B shows Serum CTX measured by ELISA in mice as described in (FIG. 13A).

FIGS. 14A-14C show relative gene expression of Wnt1 (FIG. 14A), Ptgs2 (FIG. 14B), and Tnfrsf11b (FIG. 14C) in MLO-Y4 cells treated with 10 μM of the labeled compounds for 2 hours. *p<0.05 using t-test compared to Veh. #, p<0.05 using t-test compared to Yoda1.

FIGS. 15A-15C show relative gene expression of Ptgs2 (FIG. 15A and FIG. 15B) and Wnt1 (FIG. 15C) in MLO-Y4 cells treated with 10 μM of the labeled compounds for 2 hours. *p<0.05 using t-test compared to Vehicle.

FIG. 16 shows intracellular calcium concentration in control (sh-Luc) or Piezo1 knock-down (sh-Piezo1) MLO-Y4 cells treated with 10 μM of the labeled compounds.

FIG. 17A shows dose dependent induction of intracellular calcium concentration in control (sh-Luc) or Piezo1 knock-down (sh-Piezo1) MLO-Y4 cells by the labeled compounds.

FIG. 17B shows dose dependent stimulation of ATP production in MLO-Y4 cells treated with the labeled compounds for 30 minutes.

FIGS. 18A-18B show Yoda1 and 170 increase mitochondria oxygen consumption rate (FIG. 18A) and ATP production (FIG. 18B) in osteoblastic cells. *, p<0.05

FIGS. 19A-19B show Yoda1 and 170 increase mitochondria staining in osteoblastic cells and that this effect requires Piezo1. *, p<0.05

### DETAILED DESCRIPTION OF THE INVENTION

Disclosed herein are Piezo1 agonists and methods of making and using the same. Piezo1 agonists may be used to promote tissue anabolism. As a result, the Piezo1 agonists disclosed herein may be used to chemically, rather than mechanically, stimulate cells having Piezo1 receptors.

As demonstrated in the Examples that follow, we identify Piezo1 calcium channels are involved in mechanosensation in osteocytes. Piezo1 is a mechanosensitive ion channel that is highly expressed in osteocytes. Its expression and activity are increased by mechanical stress. In addition, conditional deletion of Piezo1 in osteoblasts and osteocytes decreased cortical thickness and cancellous bone volume. Moreover, the skeletal response to anabolic loading was significantly blunted in mice lacking Piezo1 in osteoblasts and osteocytes. Importantly, administration of Piezo1 agonists increase bone mass in vivo. As a result, Piezo1 agonists chemically mimic mechanical loading and allow for the administration of pharmaceutical compositions to promote tissue anabolism.

### Piezo1 Agonists and Methods for Stimulating Anabolism in a Subject

Methods for stimulating anabolism in a subject with the use of Piezo1 agonists are provided. Suitably the method for stimulating tissue anabolism in a subject comprises administering to the subject an effective amount of a Piezo1 agonist or a pharmaceutical composition comprising the effective amount of the Piezo1 agonist.

The Piezo ion channel family consists of two members, Piezo1 and Piezo2. While Piezo2 is expressed predominately in neurons, Piezo1 is mainly expressed in non-neuronal cells (Murthy et al., 2017). Piezo1, a mechanosensitive ion channel, is highly expressed in osteocytes. A “Piezo1 agonist” is a compound that is capable of binding to Piezo1 and activating a biological response as a result. Suitably the biological response is tissue anabolism such as bone anabolism. “Anabolism” is a process of forming larger molecules via smaller molecules. Anabolic processes build tissues and organs. These processes can result in growth and/or cell differentiation. The tissue anabolism may be bone anabolism that may be characterized by an increase in bone mass, bone mineralization, bone strength, bone density, healing of bone in a subject, or any combination thereof.

As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with the Piezo1 agonist compounds disclosed herein. For example, a “subject in need of treatment” may include a subject in need of treatment to prevent or reverse loss of bone mass, bone mineral content, or bone strength.

In some embodiments, the tissue comprises cells expressing Piezo1. In some embodiments, the cells expressing Piezo1 are osteoblasts or osteocytes. “Osteoblasts” are cells with a single nucleus that synthesize bone by secreting the matrix necessary for bone formation. Osteoblasts are involved in the creation and mineralization of bone tissue. “Osteocytes” are star-shaped bone cells. They are the most commonly found cells in mature bone tissue. An osteocyte forms when an osteoblast becomes embedded in the matrix it has secreted.

In some embodiments, the tissue is a bone tissue. “Bone tissue” is a hard tissue, a type of dense connective tissue. Bone tissue contains different types of bone cells, such as osteoblasts, osteocytes, and osteoclasts. Osteoblasts and osteocytes are involved in the formation and mineralization of the bone, osteoclasts are involved in the resorption of bone tissue. Bone tissue is a mineralized tissue of two types, cortical bone and cancellous bone. The “bone” is a composite material that contains minerals bound to a matrix. The matrix is mostly composed of elastic collagen fibers. The minerals of the bone are small crystals containing calcium and phosphate, called hydroxyapatite.

In some embodiments, the subject is in need of a treatment to prevent or reverse loss of bone mass, bone mineral content, or bone strength. “Bone mass” is the measurement of the amount of minerals (mostly calcium and phosphorous) contained in a certain volume of bone. “Bone mineral content,” “bone density,” or “bone mineral density” may be used interchangeably and refer to the amount of bone mineral in bone tissue. Bone mineral content is measured as mass of mineral per volume of bone. In the clinic, bone mineral content may be measured by proxy according to optical density per square centimeter of bone surface upon imaging. “Bone strength” is the bone's resistance to fracture. It is related to, but not equivalent with, bone mineral density. Bone mineral density is a strong predictor of fracture, but there are also other factors, such as bone structure, bone remodeling, and bone quality that can be considered.

In some embodiments, the subject displays or is predisposed to developing a bone-depletive disorder. A “bone-depletive disorder” is a disease that causes bones to become brittle and more likely to fracture. Non limiting examples of bone-depletive disorders are senile osteoporosis, postmenopausal osteoporosis, steroid-induced osteoporosis, low bone-turnover osteoporosis, osteopenia, or osteomalacia.

In some embodiments, the subject is in need of a bone healing treatment. A “bone healing treatment” is a process of rebuilding a fractured bone. The bone healing process has three overlapping stages: inflammation, bone production, and bone remodeling. Inflammation starts immediately after the bone is fractured and lasts for several days. This provides the initial structural stability and framework for producing new bone. Bone production begins when the clotted blood formed by inflammation is replaced with fibrous tissue and cartilage (known as soft callus). As healing progresses, the soft callus is replaced with hard bone (known as hard callus). In bone remodeling, bone continues to form and becomes compact, returning to its original shape. In addition, blood circulation in the area improves. Once adequate bone healing has occurred, weight bearing (such as standing or walking) encourages bone remodeling.

In some embodiments, the subject has a bone fracture. A “bone fracture” is a medical condition in which there is a partial or complete break in the continuity of the bone. A bone fracture may be the result of high force impact or stress, or a minimal trauma injury which is the result of certain medical conditions that weaken the bones, such as osteoporosis, osteopenia, bone cancer, or osteogenesis imperfecta, where the fracture is termed a pathological fracture.

As used herein the term “effective amount” refers to the amount or dose of the compound, upon single or multiple dose administration to the subject, which provides the desired effect in the subject under diagnosis or treatment. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

The disclosed methods may include administering an effective amount of the disclosed compounds (e.g., as present in a pharmaceutical composition) for treating a bone-depletive disorder. Treating a bone-depletive disorder may include preventing or reversing loss of bone mass, bone mineral content, or bone strength.

The disclosed methods may also include administering an effective amount of the disclosed compounds (e.g., as present in a pharmaceutical composition) for bone healing. Bone healing may include promoting or accelerating the process of rebuilding a fractured bone.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

### Chemically Mimicking Mechanical Stimulation of a Cell Expressing Piezo1

Another aspect of the invention is methods for chemically mimicking mechanical stimulation of a cell expressing Piezo1 with the use of a Piezo1 agonist. Suitably the method for chemically mimicking mechanical stimulation of a cell expressing Piezo1 comprises contacting the cell expressing Piezo1 with an effective amount of a Piezo1 agonist.

As used herein, “chemically mimicking mechanical stimulation” refers to the ability of a compound to reproduce the effect of mechanical stimulation on a cell expressing Piezo1. In some embodiments, chemically mimicking the mechanical stimulation of Piezo1 includes an increase in intracellular calcium concentration, an increase in expression of Piezo1, an increase in expression of Ptgs2, an increase in expression of Wnt1, an increase in expression of Tnfrsf11b, an increase in expression of Cyr61, an increase in expression of Cox-2, a decrease in expression of Sost, or any combination thereof.

In some embodiments, the method increases intracellular calcium concentration. Different tissues contain calcium in different concentrations. Within a typical cell, the intracellular concentration of ionized calcium is roughly 100 nM, but is subject to increases during various cellular functions. In bone cells, for example, intracellular calcium concentration is activated by strain, pressure and/or fluid flow. Suitably, the effective amount of the Piezo1 agonist capable of increasing the intracellular calcium concentration by at least 50%, 100%, 150%, 200%, 250%, 300%, 350%, or at least 400%.

In some embodiments, the method increases the expression of a gene associated with tissue anabolism, suitably bone anabolism. Exemplary genes include, without limitation, Piezo1, Ptgs2, Wnt1, Tnfrsf11b, Cyr61, Cox-2, or any combination thereof. Suitably, the effective amount of the Piezo1 agonist capable of increasing the expression of the gene by at least 20%, 40%, 60%, 80%, 100%, 150%, or at least 200%.

In some embodiments, the method decreases the expression of a gene associated with tissue anabolism, suitably bone anabolism. Exemplary genes include, without limitation, Sost. Suitably, the effective amount of the Piezo1 agonist is capable of decreasing the expression of the gene by at least 20%, 40%, 60%, or 80%.

### Piezo1 Agonists

Another aspect of the invention provides for Piezo1 agonists. In one aspect of the invention, the Piezo1 agonists have the formula

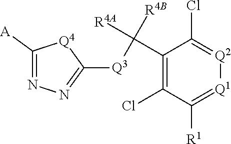

In some embodiments, the agonist substituents Q¹ and Q² may be CR² and CR³, CR² and N, or N and CR³, respectively.

When Q¹ and Q² are CR² and CR³, respectively, R² and R³ are independently selected from hydrogen, a halogen, NO₂, a C₁-C₆ haloalkyl, a C(O)OR⁶, or a C₁-C₆ alkoxy. R⁶ may be independently selected from hydrogen or a C₁-C₆ alkyl. In some embodiments when Q¹ and Q² are CR² and CR³, respectively, R¹, R², and R³ are not each hydrogen if R^{4A} and R^{4B} are each hydrogen, Q³ and Q⁴ are each S, and A is pyrazin-2-yl. In particular embodiments, the compound is not 2-((2,6-dichlorobenzyl)thio)-5-(pyrazin-2-yl)-1,3,4-thiadiazole. Compounds of this type may be prepared from the reaction of thiolates with haloaryls (e.g., as shown in Scheme 1).

When Q¹ and Q² are CR² and N, respectively, R² may be selected from a hydrogen, a halogen, NO₂, a C₁-C₆ haloalkyl, a C(O)OR⁶, or a C₁-C₆ alkoxy. R⁶ may be independently selected from hydrogen or a C₁-C₆ alkyl.

When Q¹ and Q² are N and CR³, respectively, R³ may be selected from a hydrogen, a halogen, NO₂, a C₁-C₆ haloalkyl, a C(O)OR⁶, or a C₁-C₆ alkoxy. R⁶ may be independently selected from hydrogen or a C₁-C₆ alkyl.

In some embodiments, R¹ may be independently selected from a hydrogen, a halogen, NO₂, a C₁-C₆haloalkyl, a C(O)OR⁶, or a C₁-C₆ alkoxy. R⁶ may be independently selected from hydrogen or a C₁-C₆ alkyl.

In some embodiments, R^{4A} and R^{4B} are independently selected from hydrogen, a C₁-C₆ alkyl, or R^{4A} and R^{4B} are an oxo group.

In some embodiments, Q³ is selected from S, O, or NH.

In some embodiments, Q⁴ is selected from S, O, or NR⁷. R⁷ may be independently selected from hydrogen, a C₁-C₆ alkyl, or an aryl.

In some embodiments, A is a substituted or unsubstituted heteroaryl having at least 1 nitrogen in the heteroaryl ring.

Exemplary compounds of Formula I include the following:

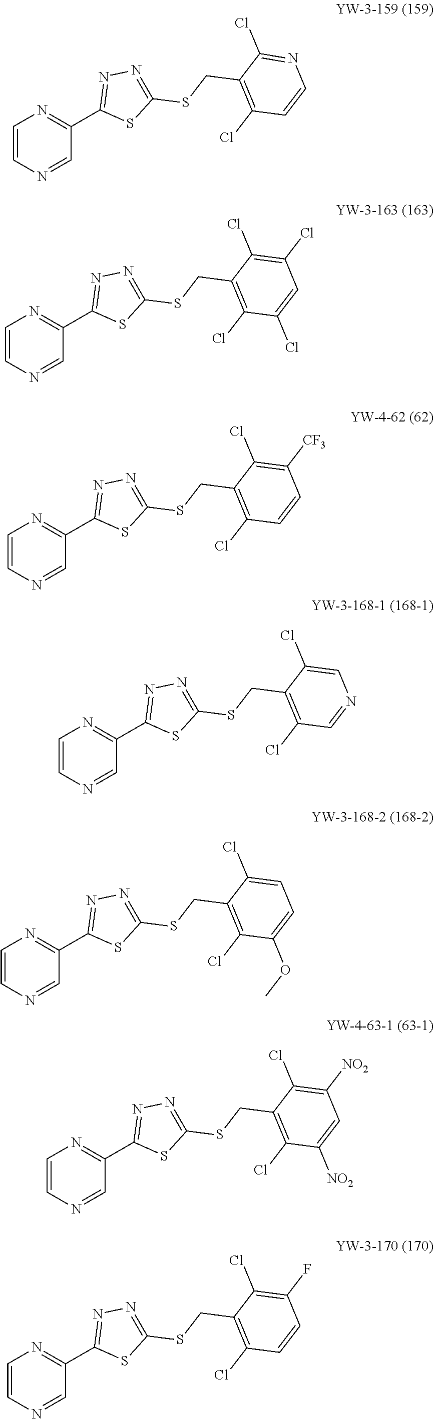

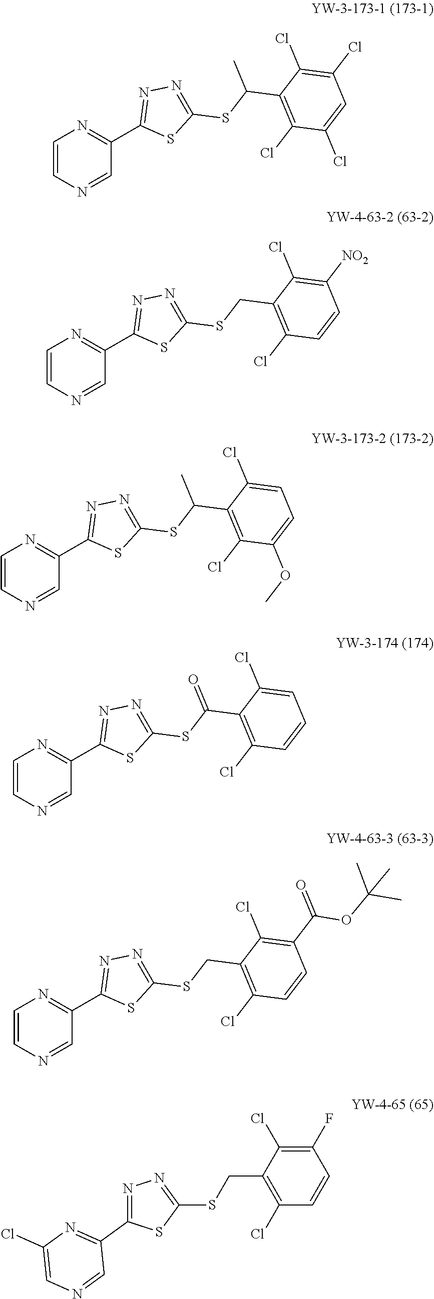 [SMILES: CC(Sc1nnc(-c2cnccn2)s1)c1c(Cl)c(Cl)cc(Cl)c1Cl ; O=[N+]([O-])c1ccc(Cl)c(CSc2nnc(-c3cnccn3)s2)c1Cl ; COc1ccc(Cl)c(C(C)Sc2nnc(-c3cnccn3)s2)c1Cl ; O=C(Sc1nnc(-c2cnccn2)s1)c1c(Cl)cccc1Cl ; CC(C)(C)OC(=O)c1ccc(Cl)c(CSc2nnc(-c3cnccn3)s2)c1Cl ; Fc1ccc(Cl)c(CSc2nnc(-c3cncc(Cl)n3)s2)c1Cl]

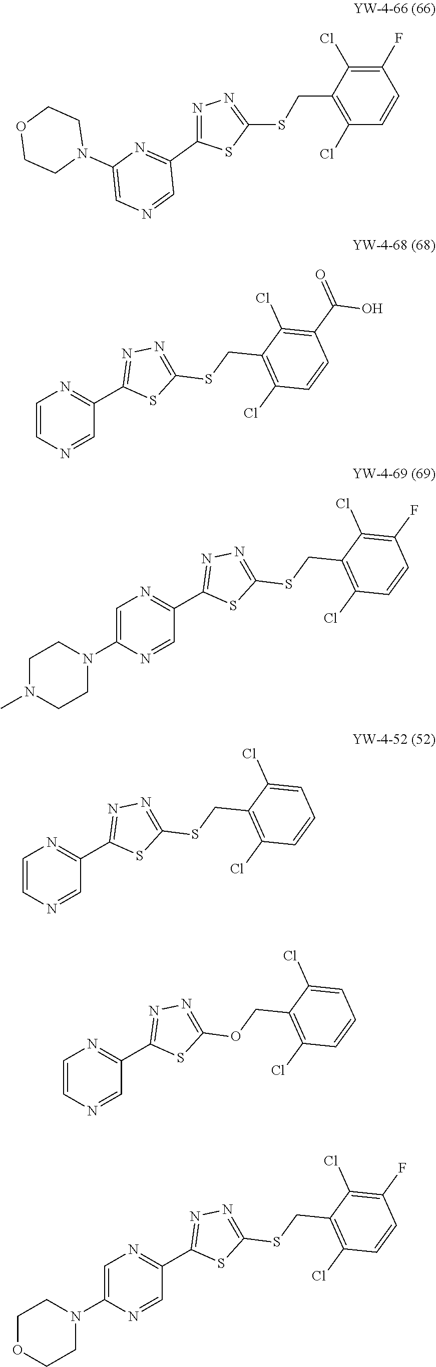 [SMILES: Fc1ccc(Cl)c(CSc2nnc(-c3cncc(N4CCOCC4)n3)s2)c1Cl ; O=C(O)c1ccc(Cl)c(CSc2nnc(-c3cnccn3)s2)c1Cl ; CN1CCN(c2cnc(-c3nnc(SCc4c(Cl)ccc(F)c4Cl)s3)cn2)CC1 ; Clc1cccc(Cl)c1CSc1nnc(-c2cnccn2)s1 ; Clc1cccc(Cl)c1COc1nnc(-c2cnccn2)s1 ; Fc1ccc(Cl)c(CSc2nnc(-c3cnc(N4CCOCC4)cn3)s2)c1Cl]

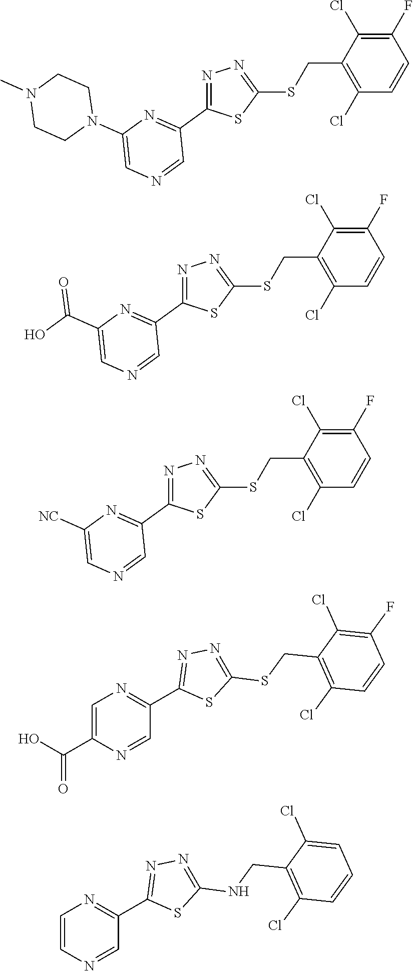 [SMILES: CN1CCN(c2cncc(-c3nnc(SCc4c(Cl)ccc(F)c4Cl)s3)n2)CC1 ; O=C(O)c1cncc(-c2nnc(SCc3c(Cl)ccc(F)c3Cl)s2)n1 ; N#Cc1cncc(-c2nnc(SCc3c(Cl)ccc(F)c3Cl)s2)n1 ; O=C(O)c1cnc(-c2nnc(SCc3c(Cl)ccc(F)c3Cl)s2)cn1 ; Clc1cccc(Cl)c1CNc1nnc(-c2cnccn2)s1]

In some embodiments, the Piezo1 agonist is one or more of YW-3-170, YW-3-159, YW-3-163, YW-4-62, and YW-4-63-2.

The compounds described herein may be prepared from the reaction of a heteroaryl compound and a haloaryl compound. For example, heteroaryl compounds may be prepared according to scheme 1.

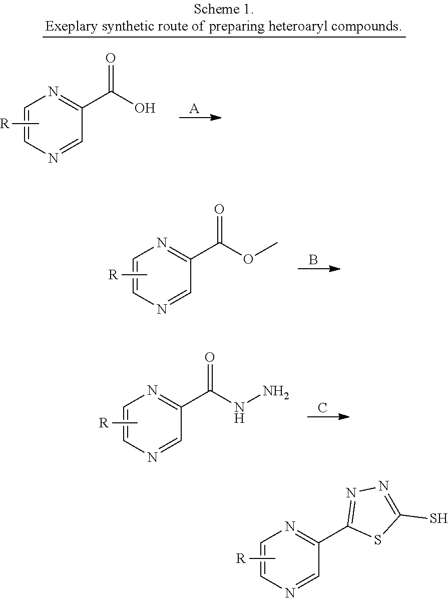

To arrive at the end product, compounds go through (A) esterification, (B) hydrazination, and (C) cyclization steps. Exemplary procedures for steps A-C, as shown on Scheme 1, are described below.

### General Procedure A: Esterification

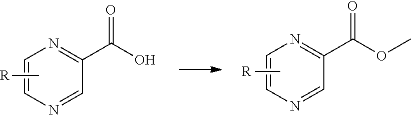

To a solution of acid (1.0 eq) and 4-dimethylaminopyridine (0.1 eq) in methanol was added 1-ethyl-3-(3-dimethylaminopropyl)carbodiimide (1.2 eq) dropwise at 0° C. under. The mixture was allowed to warm to room temperature and stirred overnight. The solution was quenched with saturated sodium bicarbonate aqueous solution (20 V). The resulting mixture was extracted with ethyl acetate. The organic layer was washed with water and brine, dried over anhydrous sodium sulfate and concentrated under vacuum. The residue was purified through flash chromatograph to get the desired product.

### General Procedure B: Hydrazination

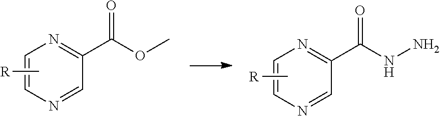

To a solution of ester (1.0 eq) in ethanol, hydrazine (1.2 eq) was added. The mixture was refluxed for 2 h. The reaction mixture was concentrated to get the desired product without further purification.

### General Procedure C: Cyclization

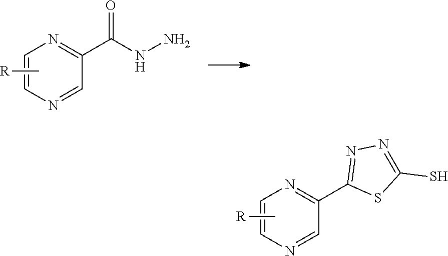

To a solution of hydrazine (1.0 eq) and potassium hydroxide (1.2 eq) in ethanol, carbon disulfide (1.2 eq) was added dropwise. The resulted slurry was stirred overnight and filtered. The residue was added to sulfuric acid and stirred at 90° C. for 2 h. After cooling to room temperature, the solution was added dropwise into ice. The precipitated solid was filtered and dissolve in sodium hydroxide aqueous solution (2 mol/L). Acetic acid was added slowly to the solution until no more solid crashed out. The slurry was filtered and the residue was obtained as the product.

Haloaryl compounds may be prepared according to scheme 2.

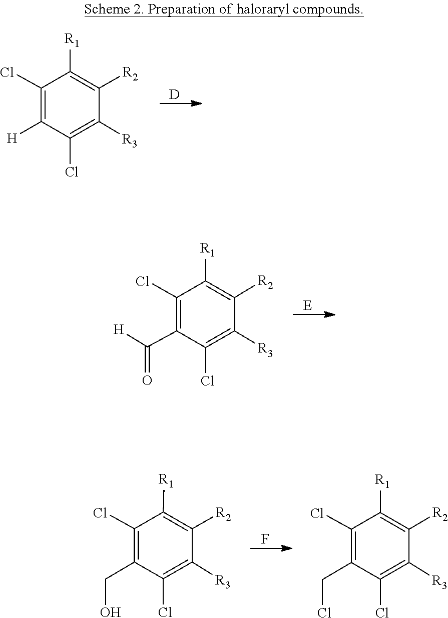

The synthesis comprises the steps of (D) acylation, (E) reduction, and (F) halogenation. The general procedures for steps D-F, as shown on Scheme 2, are described below.

### General procedure D: acylation

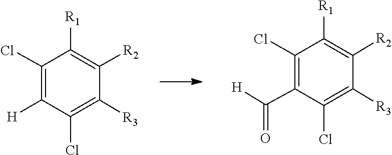

To a solution of starting material (1.0 eq) in anhydrous tetrahydrofuran, n-BuLi solution (1.1 eq) was added dropwise at −78° C. under nitrogen atmosphere. The mixture was stirred for 30 min. N,N-dimethylformamide (3.0 eq) was added to the mixture in one portion at −78° C. The solution was further stirred for 30 min and quenched with saturated sodium bicarbonate aqueous solution (20 V). The resulted mixture was extracted with ethyl acetate. The organic layer was washed with water and brine, dried over anhydrous sodium sulfate and concentrated under vacuum. The residue was purified through flash chromatograph to get the desired product.

### General Procedure E: Reduction

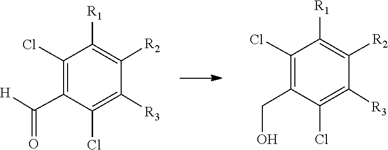

To a solution of aldehyde (1.0 eq) in methanol, sodium borohydride (1.4 eq) was added dropwise at 0° C. The mixture was allowed to warm to room temperature. After the consumption of the aldehyde was observed by thin layer chromatograph. The solution neutralized with saturated ammonium chloride aqueous solution. The resulted mixture was extracted with ethyl acetate. The organic layer was washed with water and brine, dried over anhydrous sodium sulfate and concentrated under vacuum. The residue was purified through flash chromatograph to get the desired product.

### General Procedure F: Halogenation

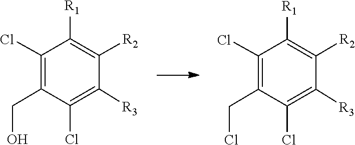

To a solution of alcohol (1.0 eq) and trimethylamine (2.0 eq) in DCM, methanesulfonyl chloride (1.5 eq) was added dropwise at 0° C. The mixture was allowed to warm to room temperature. After the consumption of the alcohol was observed by thin layer chromatography. The solution was extracted with ethyl acetate. The organic layer was washed with water and brine, dried over anhydrous sodium sulfate and concentrated under vacuum. The residue was purified through flash chromatograph to get the desired product.

To arrive at the Piezo1 agonist compounds the haloaryl and heteroaryl compounds are combined. Scheme 3 exemplifies the methodology of making these compounds.

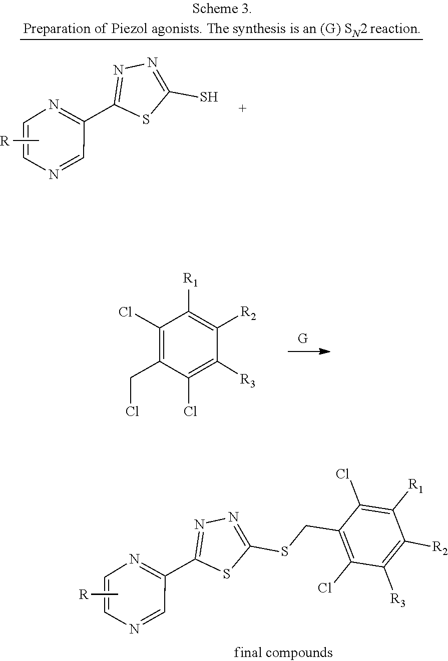

The general procedure G, from Scheme 3, is described below.

### General Procedure G: Synthesis of Final Compounds

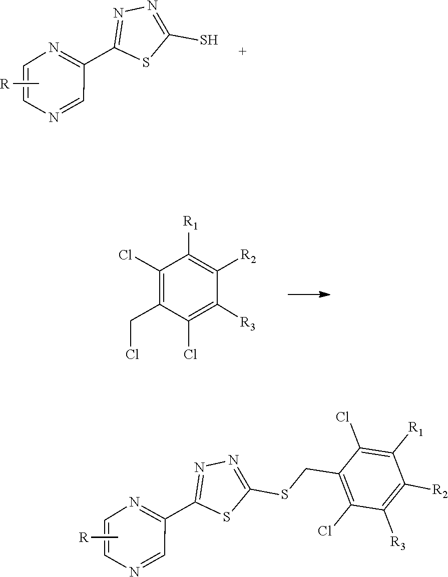

A mixture of thiol (1.0 eq), chloride/bromide (1.0 eq) and potassium carbonate (2.0 eq) in acetone was refluxed for 2 h. After the consumption of the starting materials was observed by thin layer chromatograph. The solution was extracted with ethyl acetate. The organic layer was washed with water and brine, dried over anhydrous sodium sulfate and concentrated under vacuum. The residue was purified through flash chromatograph to get the desired product.

### Pharmaceutical Compositions

The compounds utilized in the methods disclosed herein may be formulated as pharmaceutical compositions that include: (a) a therapeutically effective amount of one or more Piezo1 agonist; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods of disclosed herein may be administered as a single compound or a combination of compounds. For example, a compound that promotes Piezo1 activity may be administered as a single compound or in combination with another compound that promotes Piezo1 or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

### Definitions

As used herein, an asterick “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like

The term “heteroalkyl” as used herein refers to an “alkyl” group in which at least one carbon atom has been replaced with a heteroatom (e.g., an O, N, or S atom). One type of heteroalkyl group is an “alkoxyl” group

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a C₅-C₁₄, C₅-C₁₂, C₅-C₈, or C₅-C₆ membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O—alkyl, —O—alkenyl, —O—alkynyl, and the like.

An “epoxide” is a cyclic ether with a three-atom ring typically include two carbon atoms and whose shape approximates an isosceles triangle. Epoxides can be formed by oxidation of a double bound where the carbon atoms of the double bond form an epoxide with an oxygen atom.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —R¹C(O)N(R²)—, —R¹C(O)N(R²)R³—, —C(O)N R²R³, or —C(O)NH₂, wherein R₁, R² and R³ are independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.)

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The present disclosure is not limited to the specific details of construction, arrangement of components, or method steps set forth herein. The compositions and methods disclosed herein are capable of being made, practiced, used, carried out, and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

No admission is made that any reference, including any non-patent or patent document cited in this specification, constitutes prior art. In particular, it will be understood that, unless otherwise stated, reference to any document herein does not constitute an admission that any of these documents form part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Synthetic Methods

The disclosed compounds can be synthesized according to scheme 4 shown below.

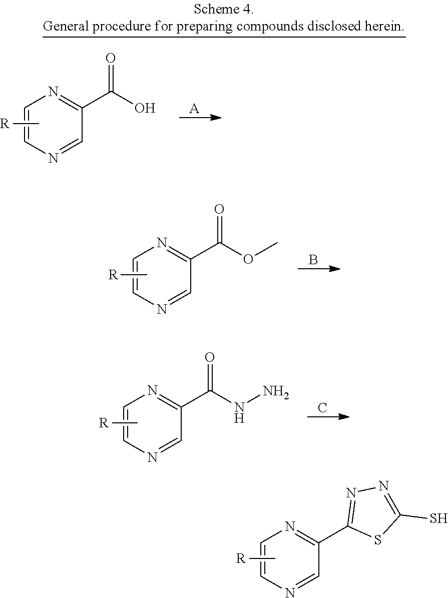

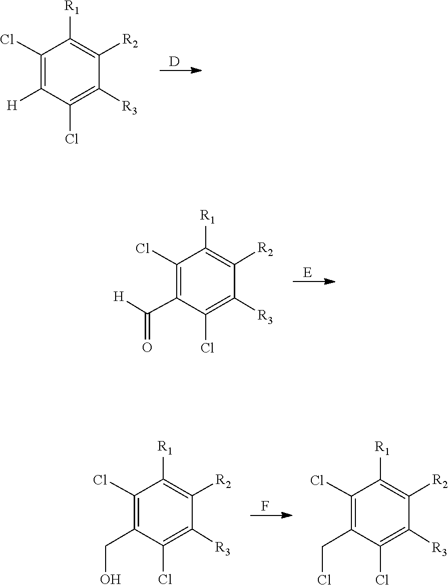

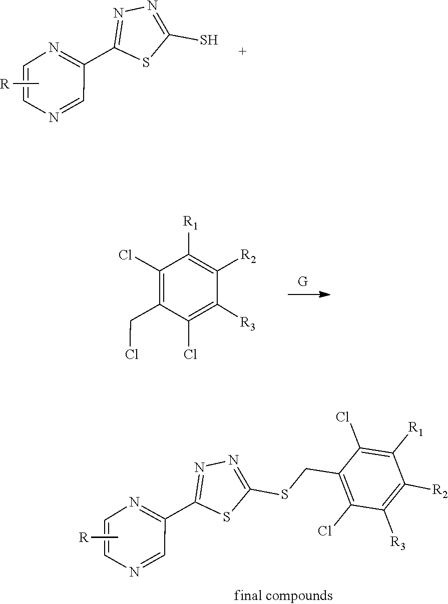

The general procedure for steps A-G shown in Scheme 4 is described below.

### General Procedure A: Esterification

To a solution of acid (1.0 eq) and 4-dimethylaminopyridine (0.1 eq) in methanol was added 1-ethyl-3-(3-dimethylaminopropyl)carbodiimide (1.2 eq) dropwise at 0° C. under. The mixture was allowed to warm to room temperature and stirred overnight. The solution was quenched with saturated sodium bicarbonate aqueous solution (20 V). The resulting mixture was extracted with ethyl acetate. The organic layer was washed with water and brine, dried over anhydrous sodium sulfate and concentrated under vacuum. The residue was purified through flash chromatograph to get the desired product.

### General Procedure B: Hydrazination

To a solution of ester (1.0 eq) in ethanol was added hydrazine (1.2 eq). The mixture was refluxed for 2 h. The reaction mixture was concentrated to get the desired product without further purification.

### General Procedure C: Cyclization

To a solution of hydrazine (1.0 eq) and potassium hydroxide (1.2 eq) in ethanol was added carbon disulfide (1.2 eq) dropwise. The resulted slurry was stirred overnight and filtered. The residue was added to sulfuric acid and stirred at 90° C. for 2 h. After cooled to room temperature, the solution was added dropwise into ice. The precipitated solid was filtered and dissolve in sodium hydroxide aqueous solution (2 mol/L). Acetic acid was added slowly to the solution until no more solid crashed out. The slurry was filtered and the residue was obtained as the product.

### General Procedure D: Acylation

To a solution of starting material (1.0 eq) in anhydrous tetrahydrofuran was added n-BuLi solution (1.1 eq) dropwise at −78° C. under nitrogen atmosphere. The mixture was stirred for 30 min. N,N-dimethylformamide (3.0 eq) was added to the mixture in one portion at −78° C. The solution was further stirred for 30 min and quenched with saturated sodium bicarbonate aqueous solution (20 V). The resulted mixture was extracted with ethyl acetate. The organic layer was washed with water and brine, dried over anhydrous sodium sulfate and concentrated under vacuum. The residue was purified through flash chromatograph to get the desired product.

### General Procedure E: Reduction

To a solution of aldehyde (1.0 eq) in methanol was added sodium borohydride (1.4 eq) dropwise at 0° C. The mixture was allowed to warm to room temperature. After the consumption of the aldehyde was observed by thin layer chromatograph. The solution neutralized with saturated ammonium chloride aqueous solution. The resulted mixture was extracted with ethyl acetate. The organic layer was washed with water and brine, dried over anhydrous sodium sulfate and concentrated under vacuum. The residue was purified through flash chromatograph to get the desired product.

### General Procedure F: Halogenation

To a solution of alcohol (1.0 eq) and trimethylamine (2.0 eq) in DCM was added methanesulfonyl chloride (1.5 eq) dropwise at 0° C. The mixture was allowed to warm to room temperature. After the consumption of the alcohol was observed by thin layer chromatograph. The solution was extracted with ethyl acetate. The organic layer was washed with water and brine, dried over anhydrous sodium sulfate and concentrated under vacuum. The residue was purified through flash chromatograph to get the desired product.

### General Procedure G: Synthesis of Final Compounds

A mixture of thiol (1.0 eq), chloride/bromide (1.0 eq) and potassium carbonate (2.0 eq) in acetone was refluxed for 2 h. After the consumption of the starting materials was observed by thin layer chromatograph. The solution was extracted with ethyl acetate. The organic layer was washed with water and brine, dried over anhydrous sodium sulfate and concentrated under vacuum. The residue was purified through flash chromatograph to get the desired product.

### Synthetic Example A. Preparation of 2-(pyrazin-2-yl)-5-((2,3,5,6-tetrachlorobenzyl) thio)-1,3,4-thiadiazole

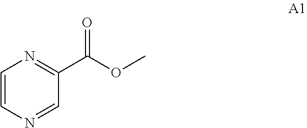

Intermediate A1 was prepared from pyrazine-2-carboxylic acid following general procedure A, beige solid (yield 90%).

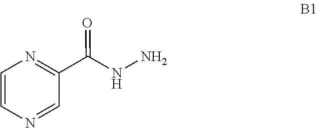

Intermediate B1 was prepared from A1 following general procedure B, yellow solid (yield 99%).

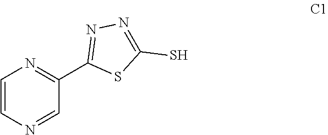 [SMILES: Sc1nnc(-c2cnccn2)s1]

Intermediate C1 was prepared from B1 following general procedure C, yellow solid (yield 40%). ¹H NMR (400 MHz, DMSO-d₆) δ 9.26 (d, J=1.5 Hz, 1H), 8.80 (d, J=2.6 Hz, 1H), 8.75 (dd, J=2.5, 1.6 Hz, 1H).

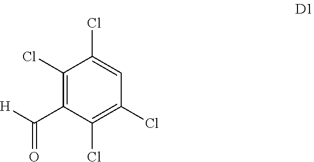 [SMILES: O=Cc1c(Cl)c(Cl)cc(Cl)c1Cl]

Intermediate D1 was prepared from 1,2,4,5-tetrachlorobenzene following general procedure D, white solid (yield 80%). ¹H NMR (400 MHz, CDl₃) δ 10.36 (s, 1H), 7.77 (s, 1H).

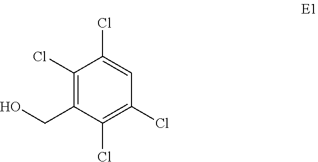 [SMILES: OCc1c(Cl)c(Cl)cc(Cl)c1Cl]

Intermediate E1 was prepared from D1 following general procedure E, white solid (yield 73%).

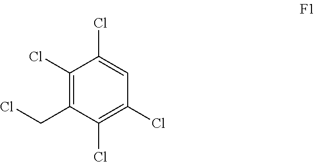 [SMILES: ClCc1c(Cl)c(Cl)cc(Cl)c1Cl]

Intermediate F1 was prepared from E1 following general procedure F, off-white solid (yield 74%). 1H NMR (400 MHz, CDl₃) δ 7.62 (s, 1H), 4.92 (s, 2H).

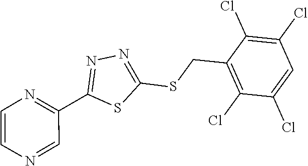 [SMILES: Clc1cc(Cl)c(Cl)c(CSc2nnc(-c3cnccn3)s2)c1Cl]

The above compound was prepared from C1 and F1 following general procedure G, off-white solid (yield 57%). 1H NMR (400 MHz, CDl₃) δ 9.56 (d, J=1.5 Hz, 1H), 8.68 (d, J=2.5 Hz, 1H), 8.61 (dd, J=2.4, 1.7 Hz, 1H), 7.62 (s, 1H), 5.11 (s, 2H). LCMS: 423.

### Synthetic Example B. Preparation of 2-((2,6-dichloro-3-fluorobenzyl)thio)-5-(pyrazin-2-yl)-1,3,4-thiadiazole

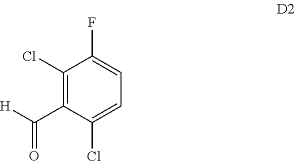 [SMILES: O=Cc1c(Cl)ccc(F)c1Cl]

Intermediate D2 was prepared from 2,4-dichloro-1-fluorobenzene following general procedure D, white solid (yield 60%). ¹H NMR (400 MHz, CDl₃) δ 10.45 (s, 1H), 7.38 (dd, J=8.9, 4.5 Hz, 1H), 7.29 (t, J=8.4 Hz, 1H).

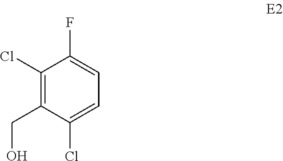 [SMILES: OCc1c(Cl)ccc(F)c1Cl]

Intermediate E2 was prepared from D2 following general procedure E, white solid (yield 80%).

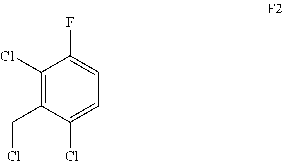 [SMILES: Fc1ccc(Cl)c(CCl)c1Cl]

Intermediate F2 was prepared from E2 following general procedure F, colorless oil (yield 94%). 1H NMR (400 MHz, CDl₃) δ 7.33 (dd, J=8.8, 4.7 Hz, 1H), 7.11 (t, J=8.5 Hz, 1H), 4.86 (s, 2H).

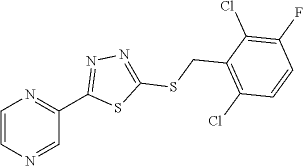 [SMILES: Fc1ccc(Cl)c(CSc2nnc(-c3cnccn3)s2)c1Cl]

The above compound was prepared from C1 and F2 following general procedure G, off-white solid (yield 51%). 1H NMR (400 MHz, CDl₃) δ 9.56 (s, 1H), 8.67 (d, J=2.3 Hz, 1H), 8.60 (s, 1H), 7.34 (dd, J=8.8, 4.5 Hz, 1H), 7.12 (t, J=8.4 Hz, 1H), 5.02 (s, 2H). LCMS: 373.

### Synthetic Example C. Preparation of 2-((2,6-dichloro-3-(trifluoromethyl)benzyl) thio)-5-(pyrazin-2-yl)-1,3,4-thiadiazole

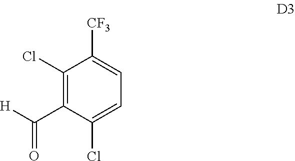 [SMILES: O=Cc1c(Cl)ccc(C(F)(F)F)c1Cl]

Intermediate D3 was prepared from 2,4-dichloro-1-(trifluoromethyl)benzene following general procedure D, pale-yellow solid (80%). 1H NMR (400 MHz, CDl₃) δ 10.47 (s, 1H), 7.79 (dd, J=8.4, 6.3 Hz, 1H), 7.56-7.49 (m, 1H).

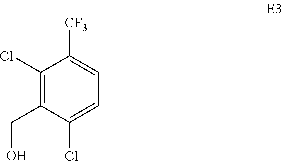 [SMILES: OCc1c(Cl)ccc(C(F)(F)F)c1Cl]

Intermediate E3 was prepared from D3 following general procedure E, white solid (83%).

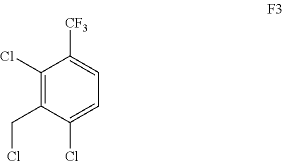 [SMILES: FC(F)(F)c1ccc(Cl)c(CCl)c1Cl]

Intermediate F3 was prepared from E3 following general procedure F, white solid (yield 90%). ¹H NMR (400 MHz, CDl₃) δ 7.63 (d, J=8.5 Hz, 1H), 7.47 (d, J=8.6 Hz, 1H), 4.93 (s, 2H).

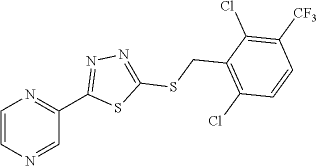 [SMILES: FC(F)(F)c1ccc(Cl)c(CSc2nnc(-c3cnccn3)s2)c1Cl]

The above compound was prepared from C1 and F3 following general procedure G, pale-yellow solid (yield 62%). ¹H NMR (400 MHz, CDl₃) δ 9.56 (d, J=1.5 Hz, 1H), 8.68 (d, J=2.5 Hz, 1H), 8.61 (dd, J=2.5, 1.6 Hz, 1H), 7.64 (d, J=8.6 Hz, 1H), 7.49 (d, J=8.5 Hz, 1H), 5.11 (s, 2H). LCMS: 423.

### Synthetic Example D. Preparation of 2-((2,6-dichloro-3-nitrobenzyl)thio)-5-(pyrazin-2-yl)-1,3,4-thiadiazole

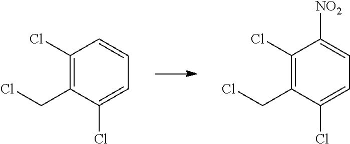 [SMILES: ClCc1c(Cl)cccc1Cl ; O=[N+]([O-])c1ccc(Cl)c(CCl)c1Cl]

To a solution of 1,3-dichloro-2-(chloromethyl)benzene (1.0 eq) in sulfuric acid (5V) was added fuming nitric acid (1.1 eq) dropwise at 40° C. The mixture was further stirred for 1 h, and the consumption of starting material was observed by thin layer chromatography. The mixture was cooled to room temperature and poured into ice. The resulting mixture was extracted with ethyl acetate. The organic layer was washed with water and brine, dried over anhydrous sodium sulfate and concentrated under vacuum. The residue was purified through flash chromatograph to get 1,3-dichloro-2-(chloromethyl)-4-nitrobenzene as colorless oil, yield 58%. ¹H NMR (400 MHz, CDl₃) δ 7.75 (d, J=8.7 Hz, 1H), 7.51 (d, J=8.8 Hz, 1H), 4.93 (s, 2H).

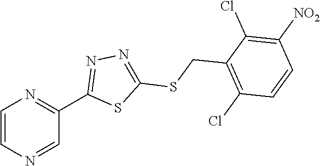 [SMILES: O=[N+]([O-])c1ccc(Cl)c(CSc2nnc(-c3cnccn3)s2)c1Cl]

The above compound was prepared from C1 and 1,3-dichloro-2-(chloromethyl)-4-nitrobenzene following general procedure G, yellow solid (yield 50%). 1H NMR (400 MHz, CDl₃) δ 9.55 (d, J=1.5 Hz, 1H), 8.68 (d, J=2.5 Hz, 1H), 8.63-8.59 (m, 1H), 7.75 (d, J=8.8 Hz, 1H), 7.52 (d, J=8.8 Hz, 1H), 5.11 (s, 2H).

### Stimulation Of Piezo1 Promotes Bone Anabolism

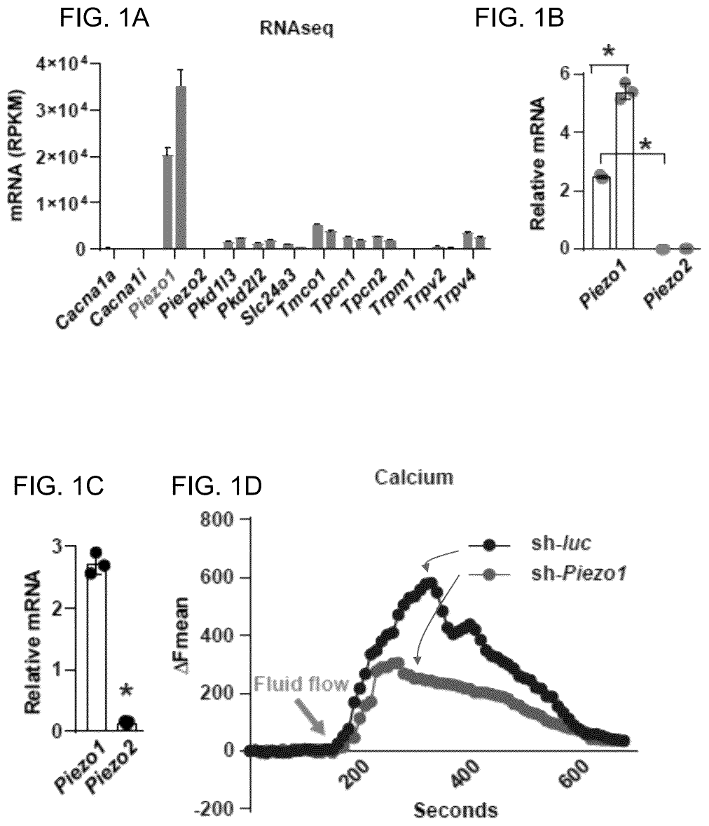

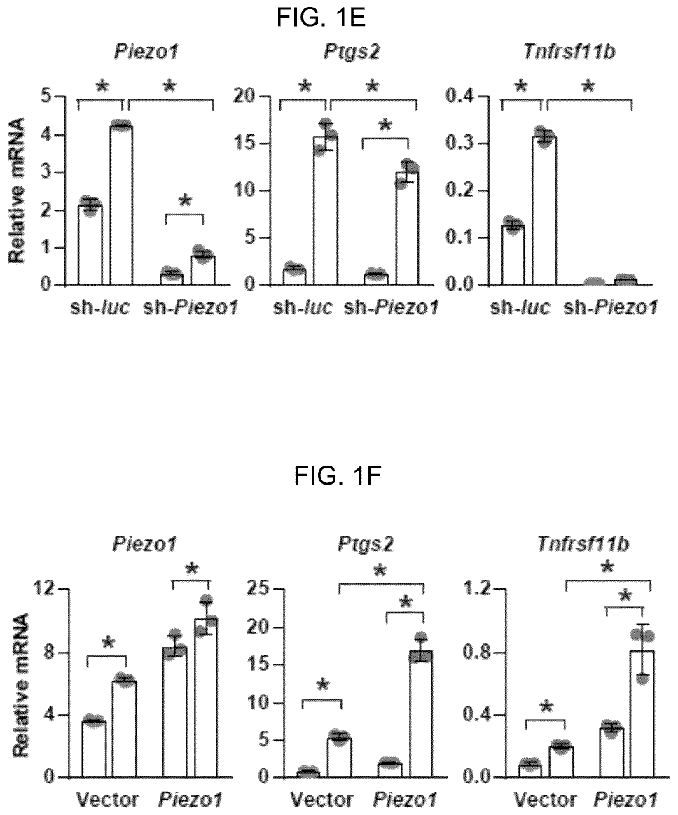

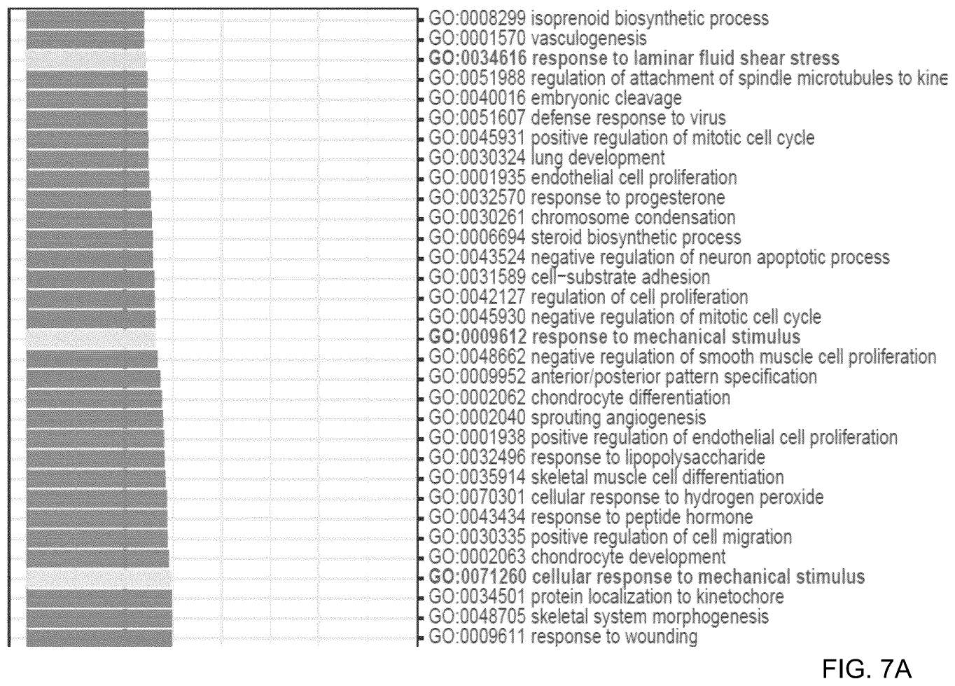

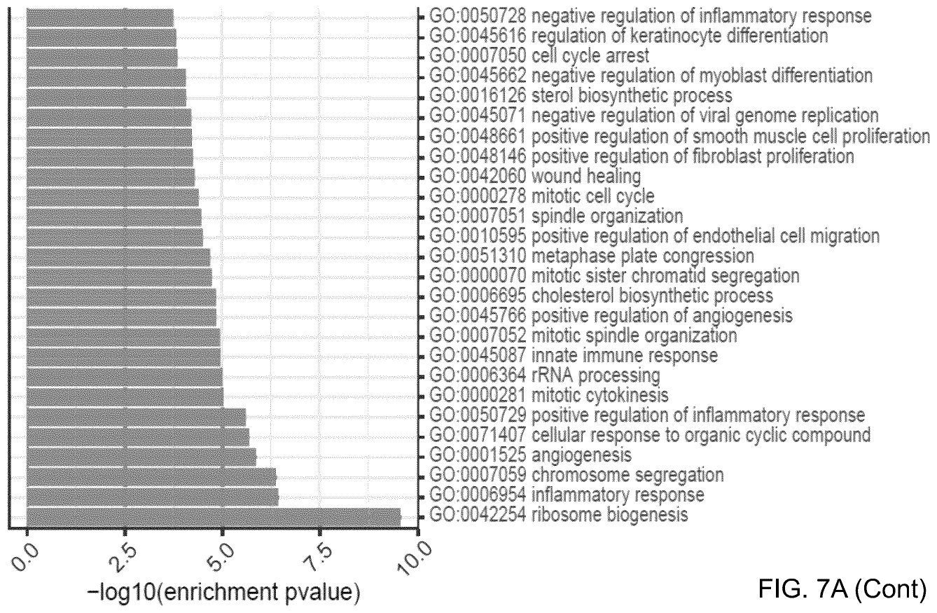

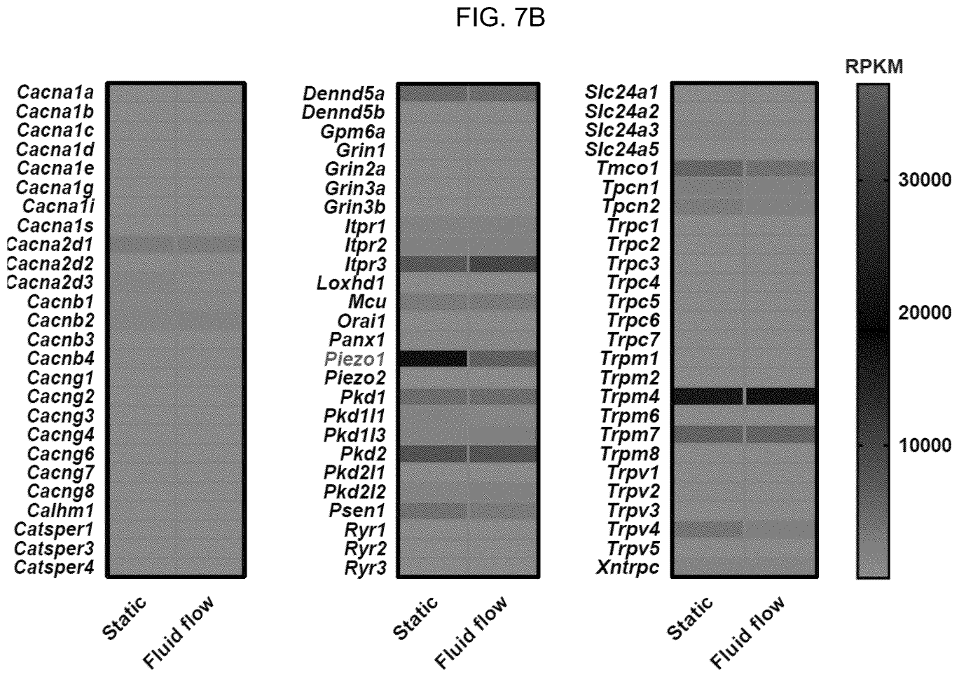

Piezo1 mediates mechanotransduction in an osteocyte cell line. To identify calcium channels that respond to mechanical signals in osteocytes, we compared gene expression profiles of the osteocytic cell line MLO-Y4 under static and fluid flow conditions by RNA-seq. Principal components analysis and volcano plot of transcripts indicated that a significant number of genes were differentially expressed in MLO-Y4 cells under static versus fluid shear stress (FIGS. 1A-1F and 6A-6B). GO-enrichment analysis revealed enrichment in genes known to respond to mechanical signals, thereby validating the fluid flow experiment (FIG. 7A). We then identified differentially expressed genes related to calcium channels. Piezo1 was the most highly expressed among 78 calcium channels detected in MLO-Y4 cells under static condition (FIG. 7B). In addition, Piezo1 was also highly up-regulated by fluid flow in MLO-Y4 cells as determined by RNA-seq (FIG. 1A) and RT-qPCR (FIG. 1B). The Piezo ion channel family consists of two members, Piezo1 and Piezo2. While Piezo2 is expressed predominately in neurons, Piezo1 is mainly expressed in non-neuronal cells (Murthy et al., 2017). Consistent with this previous evidence, the expression of Piezo1 was approximately 200-fold higher than that of Piezo2 in MLO-Y4 cells (FIG. 1B). Piezo1 expression was also much higher than Piezo1 in osteocyte-enriched cortical bone isolated from 12-week-old mice (FIG. 1C). Therefore, we focused our remaining analysis on Piezo1. Knock-down of Piezo1 mRNA in MLO-Y4 cells significantly blunted the increase in intracellular calcium induced by fluid-flow (FIG. 1D). Knock-down of Piezo1 also blunted fluid-flow stimulation of Ptgs2 and Tnfrsf11b (FIG. 1E), two well-known targets of fluid shear stress in osteocytes (Wadhwa et al., 2002, Zhao et al., 2016). Conversely, overexpression of Piezo1 in MLO-Y4 cells increased the expression of Ptgs2 and Tnfrsf11b and enhanced their induction by fluid shear stress (FIG. 1F). These results demonstrate that Piezo1 contributes to the response of MLO-Y4 cells to fluid shear stress.

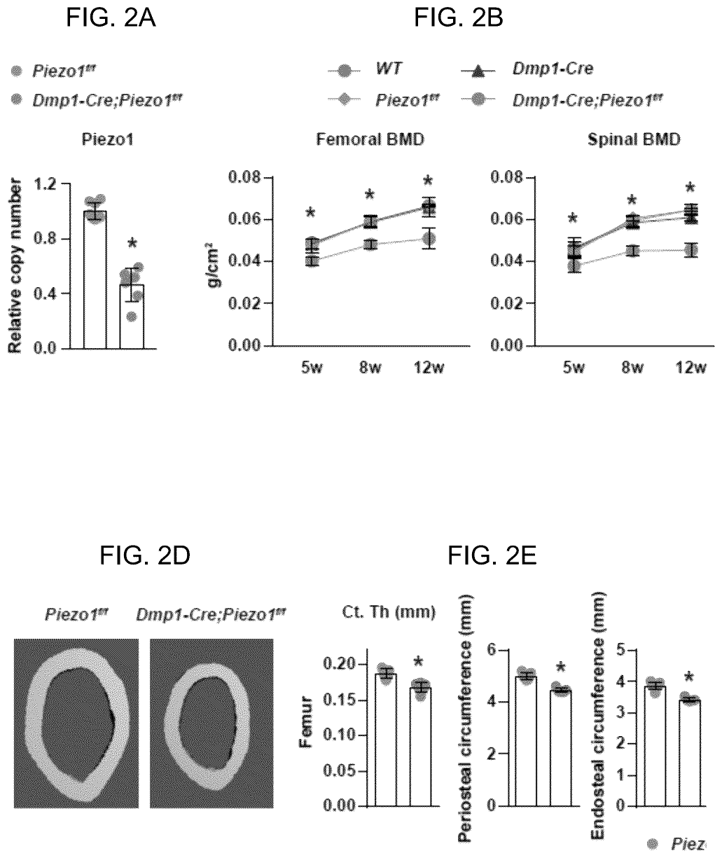

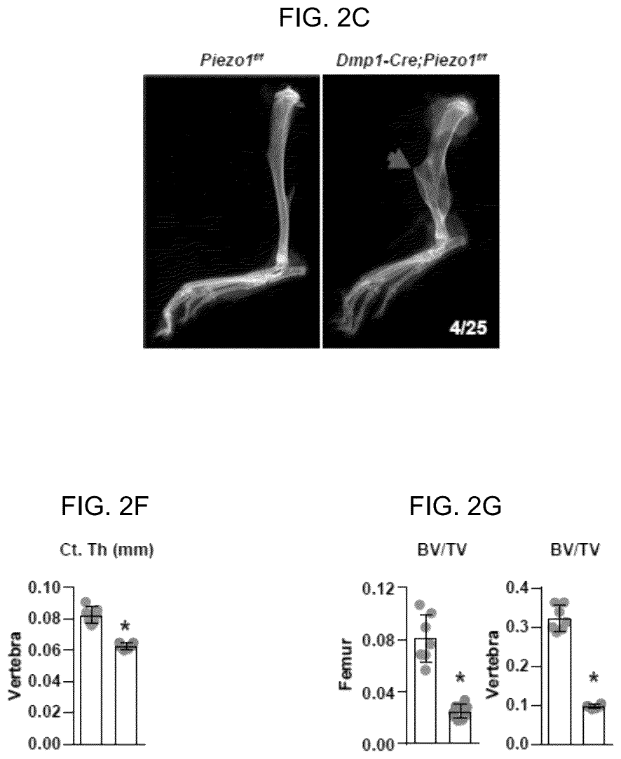

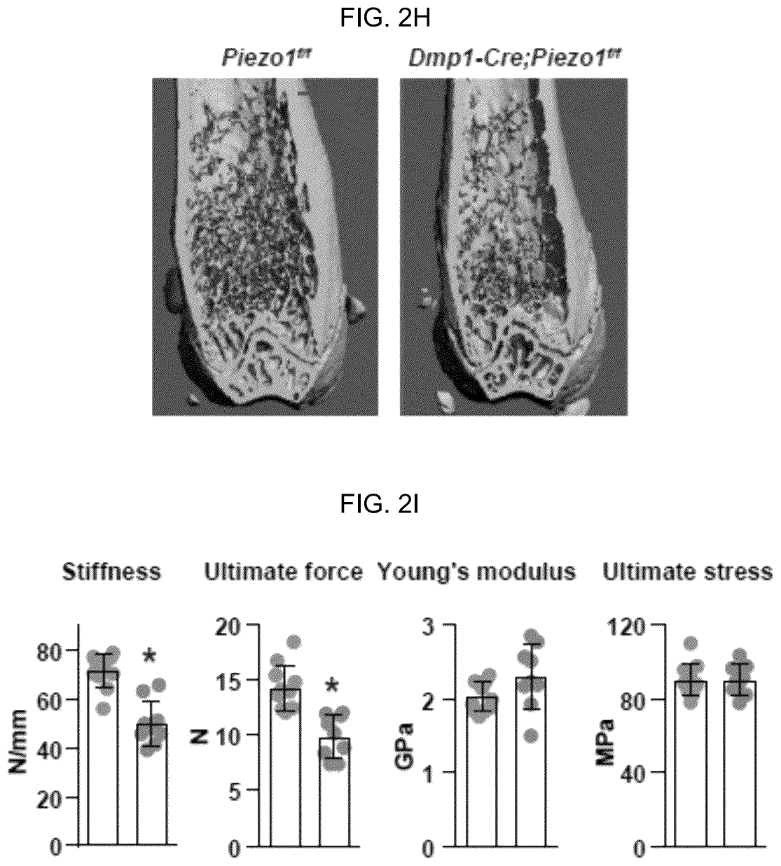

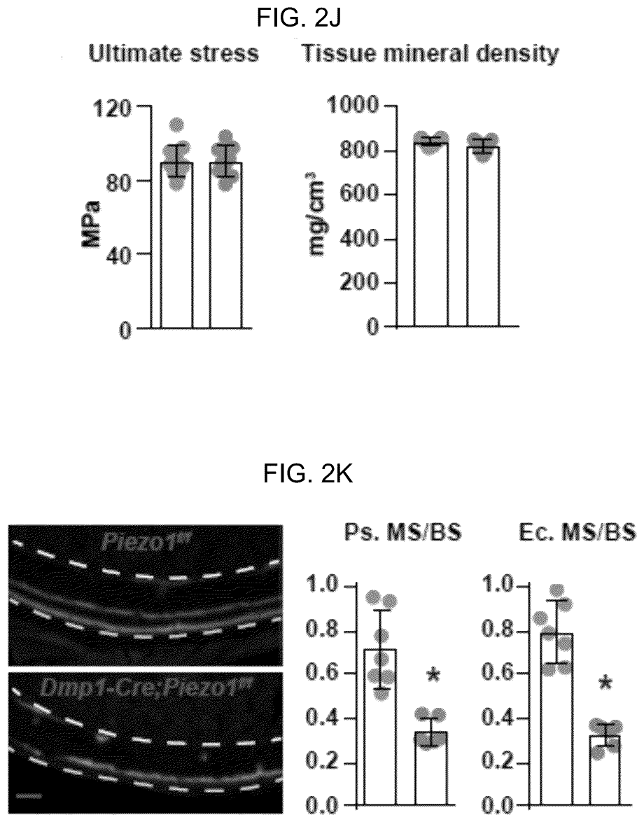

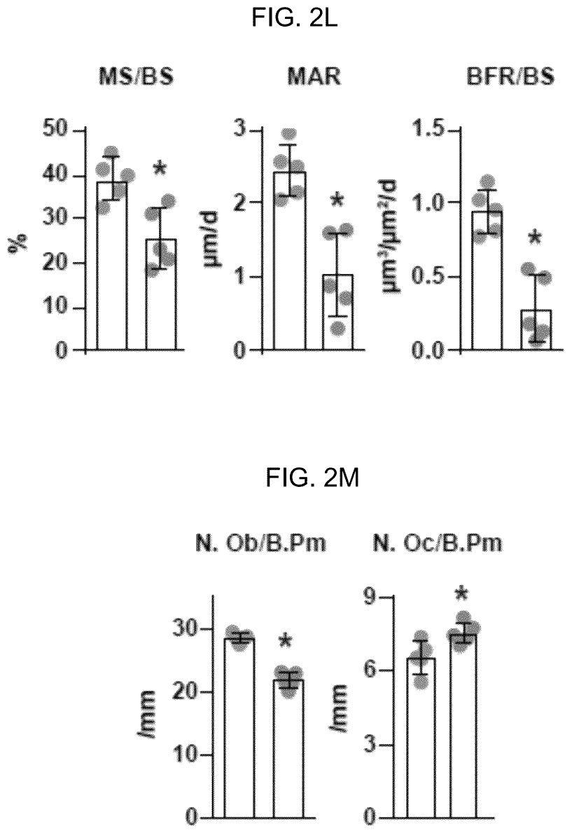

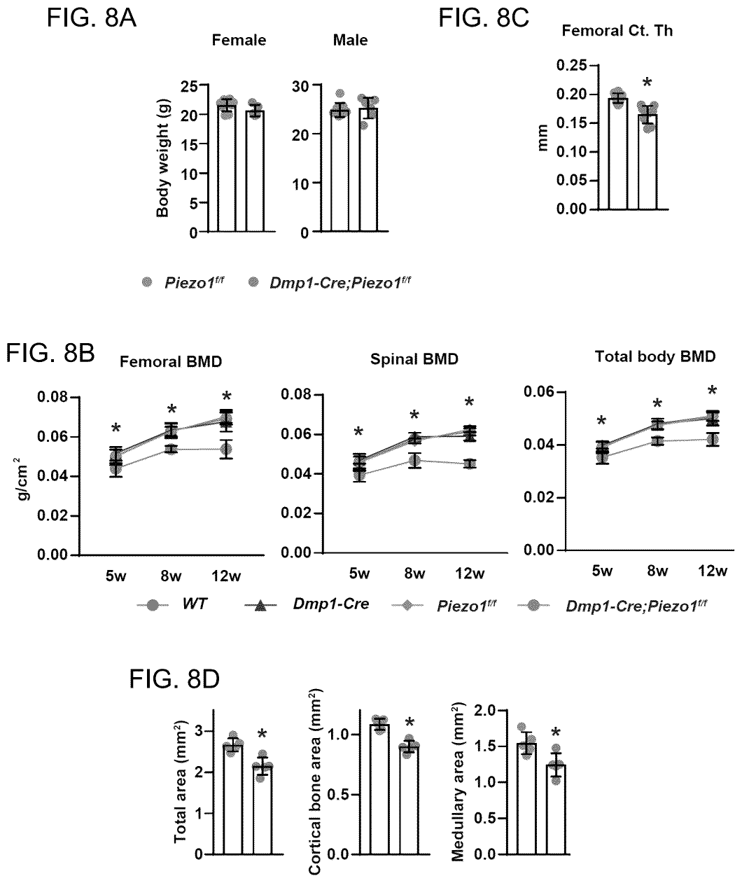

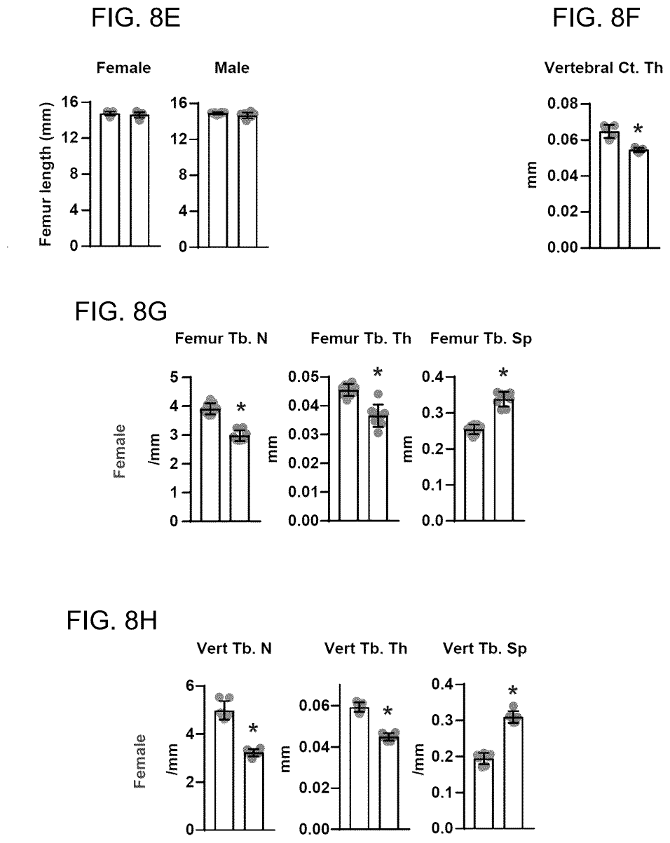

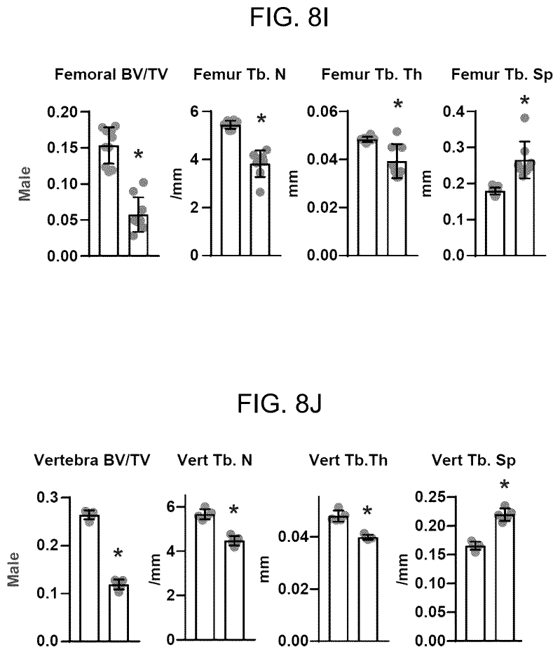

Loss of Piezo1 in osteoblasts and osteocytes decreases bone formation and bone mass. To determine the role of Piezo1 in osteocytes in vivo, we deleted Piezo1 by crossing Piezo1^{f/f} mice (Cahalan et al., 2015) with Dmp1-Cre transgenic mice, which express the Cre recombinase in osteoblasts and osteocytes (Bivi et al., 2012, Xiong et al., 2015). Deletion of the Piezo1 gene was confirmed by qPCR of genomic DNA isolated from osteocyte-enriched cortical bone (FIG. 2A). Mice lacking the Piezo1 gene in osteoblasts and osteocytes, hereafter referred to as Dmp1-Cre; Piezo1^{f/f} mice, exhibited normal body weight compared to their control Piezo1^{f/f} littermates (FIG. 8A). Both female and male Dmp1-Cre; Piezo1^{f/f} mice exhibited low bone mineral density (BMD) at 5, 8, and 12 weeks of age as measured by dual energy x-ray absorptiometry (DXA) and the difference increased as the mice matured (FIG. 2B and FIG. 8B). Since the three control groups, including wild-type (WT), Dmp1-Cre, and Piezo1^{f/f} littermates, displayed similar BMD, we used Piezo1^{f/f} littermates as controls in the remaining studies. Spontaneous fractures were observed in the tibia of conditional knockout mice at a frequency of 0.16 (FIG. 2C). Detailed analysis of the skeletal phenotype of these mice at 12 weeks of age by micro-CT revealed that femoral cortical thickness was lower in Dmp1—Cre; Piezo1^{f/f} mice compared with controls in both sexes (FIGS. 2D-2E and 8C). Periosteal and endocortical circumferences were also decreased in the femur of Dmp1-Cre; Piezo1^{f/f} mice (FIG. 2E). In line with these changes, the total cross sectional area, cortical bone area, and medullary area were reduced in the conditional knockout mice (FIG. 8D). In contrast to the changes in bone width, the length of the femurs was not different between genotypes indicating that longitudinal bone growth was normal in conditional knockout mice (FIG. 8E). A decrease in cortical bone thickness was also detected in vertebrae of Dmp1-Cre; Piezo1^{f/f} female and male mice (FIGS. 2F and 8F). Analysis of cancellous bone in the femur and vertebra revealed that bone volume over tissue volume, trabecular number, and trabecular thickness were decreased, while trabecular separation was increased in female Dmp1-Cre; Piezo1^{f/f} mice compared to their control littermates (FIGS. 2G-2H and 8G-8H). Similar results were obtained in male mice (FIGS. 8I-8J).

Biomechanical testing by 3-point bending showed that the femurs from Dmp1-Cre; Piezo1^{f/f} mice had reduced stiffness and ultimate force (FIG. 2I). However, the Young's modulus and ultimate stress did not change, suggesting that the lower strength was due to differences in size and mass rather than changes in bone material properties (FIG. 2I). Consistent with this, the tissue mineral density of femoral cortical bone was unaffected by deletion of Piezo1 (FIG. 2J).

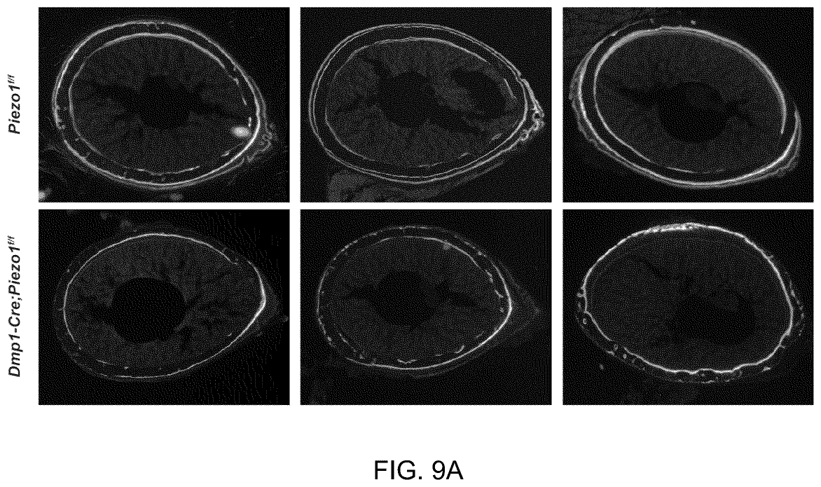

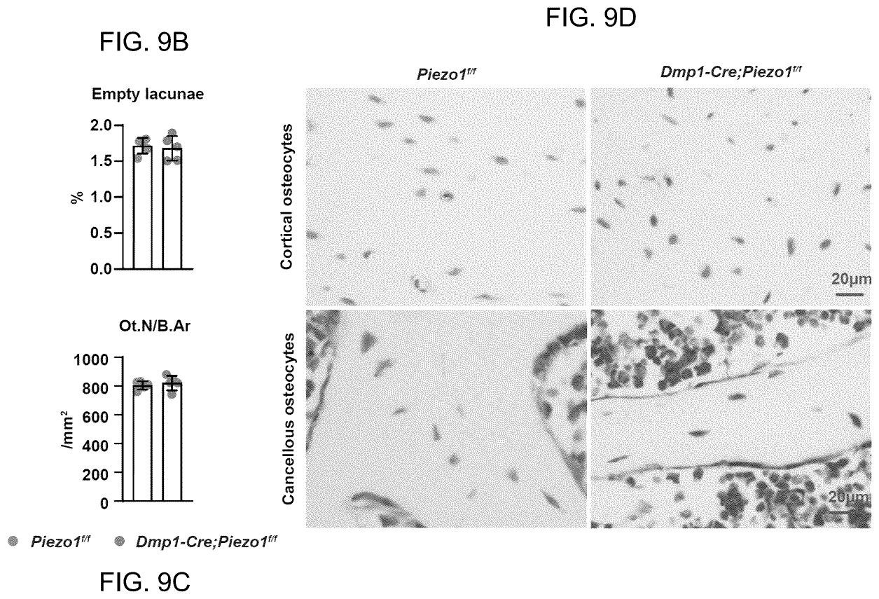

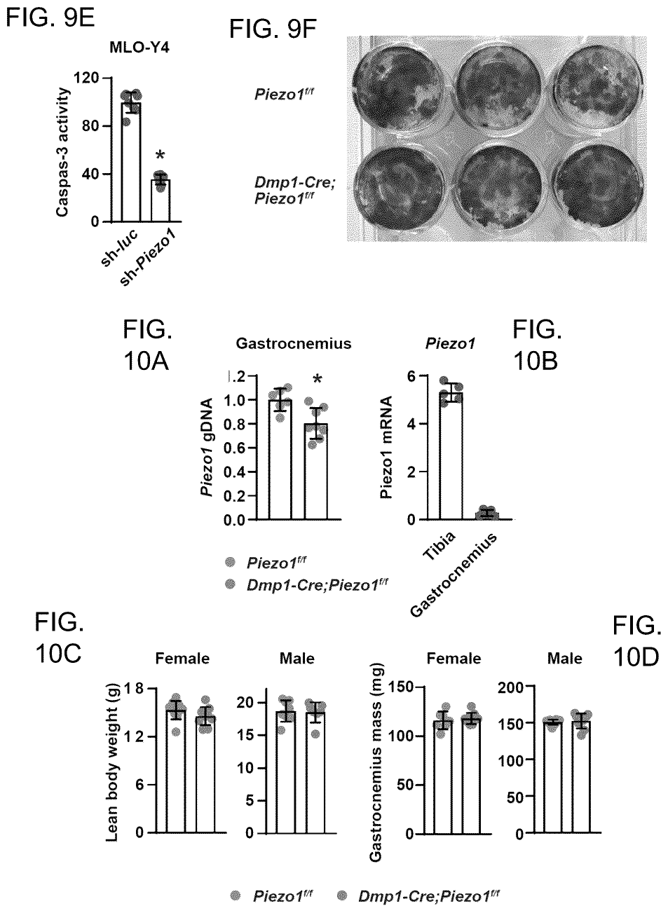

To evaluate the cellular changes underlying the skeletal phenotype of the conditional knockout mice, we performed bone histomorphometry of femoral cortical bone and found that periosteal and endocortical mineralizing surfaces were significantly reduced in Dmp1-Cre; Piezo1^{f/f} mice at 5 weeks, an age of rapid bone growth (FIG. 2K and FIG. 9A). Bone formation at the outer (periosteal) surfaces of bone is a critical process for the enlargement of the skeleton. While double labels were easily seen in control mice, double labels were not observed in the conditional knockout mice, indicating that the bone formation rate at the periosteum of Dmp1-Cre; Piezo1^{f/f} mice was extremely low. Histomorphometric analysis of vertebral trabecular bone also revealed a decrease in mineralizing surface, mineral apposition rate, and bone formation rate in the conditional knockout mice (FIG. 2L). In line with these changes, osteoblast number was lower in Dmp1-Cre; Piezo1^{f/f} mice (FIG. 2M). In addition, we observed an increase in osteoclast number in the conditional knockout mice (FIG. 2M).

To evaluate whether cell death could account for the changes seen with Piezo1 deletion, we measured the percentage of empty osteocyte lacunae and osteocyte number. We did not observe changes in the percentage of empty osteocyte lacunae or the number of osteocytes normalized to bone area in Dmp1-Cre; Piezo1^{f/f} mice compared to their littermate controls (FIG. 9B, 9C). Consistent with these results, we did not observe any apparent morphological changes in osteocytes in the conditional knockout mice (FIG. 9D). In addition, knock-down of Piezo1 in MLO-Y4 cells decreased, rather than increased, Capase3 activity (FIG. 9E). These results indicate that Piezo1 deletion does not increase osteocyte death in vitro or in vivo. We also analyzed osteoblastogenesis in vitro and found normal osteoblast differentiation of bone marrow stromal cells from Dmp1-Cre; Piezo1^{f/f} mice, as indicated by Alizarin Red staining (FIG. 9F).

Since the Dmp1-Cre transgene also leads to recombination in a sub-population of muscle cells (Lim et al., 2017), we measured Piezo1 deletion in gastrocnemius muscle, lean body weight, and gastrocnemius muscle mass to determine whether altered muscle mass could have contributed to the skeletal phenotype. We detected about 20% deletion of the Piezo1 gene in the conditional knockout mice (FIG. 10A). In addition, Piezo1 expression in gastrocnemius muscle was about times lower than that in bone (FIG. 10B). More importantly, we did not observe any difference in lean body weight or gastrocnemius muscle mass between the conditional knockout mice and 10 their control littermates (FIG. 10C, 10D). These results demonstrate that Piezo1 in osteoblasts, osteocytes, or both, is essential for normal bone size and mass.

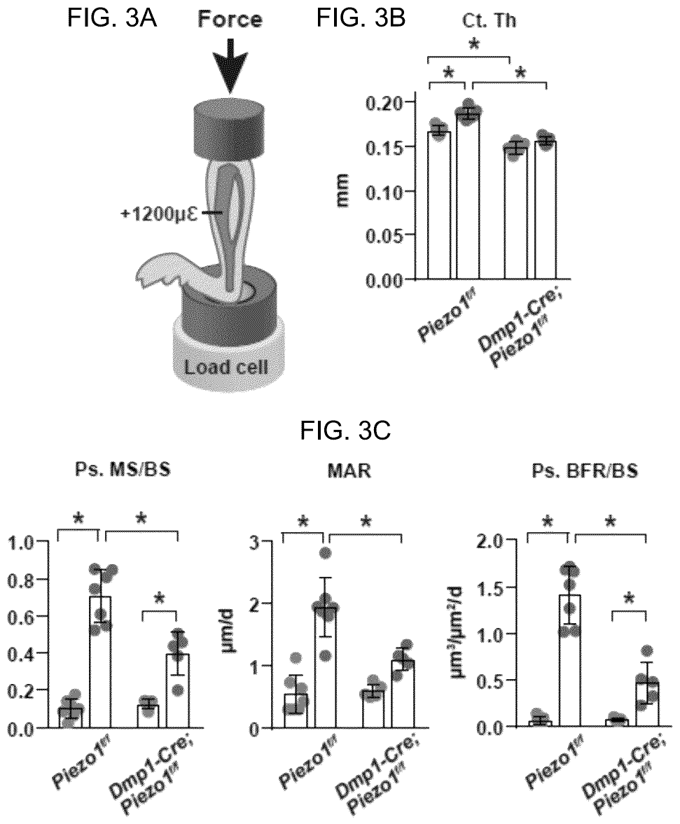

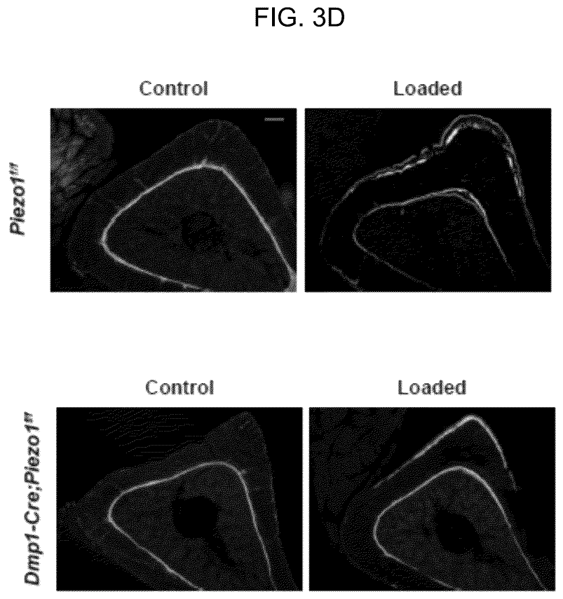

Loss of Piezo1 in osteoblasts and osteocytes blunts the skeletal response to mechanical loads. To determine whether Piezo1 in osteoblasts or osteocytes is required for the skeletal response to increased mechanical loading, we loaded the left tibia of 16-week-old female Dmp1-Cre; Piezo1^{f/f} mice and their control littermates with +1200 με peak strain at the midshaft, as illustrated in FIG. 3A. Two weeks of anabolic loading increased tibial cortical thickness in control mice but not in conditional knockout mice (FIG. 3B). Consistent with the changes in bone mass, loading increased periosteal bone formation rate in control mice, due to increases in both mineralizing surface and mineral apposition rate (FIG. 3C, 3D). The load-stimulated bone formation was significantly blunted in conditional knockout mice (FIG. 3C, 3D). These results suggest that Piezo1 in osteoblasts, osteocytes, or both, plays an essential role in the response of the skeleton to mechanical loads.

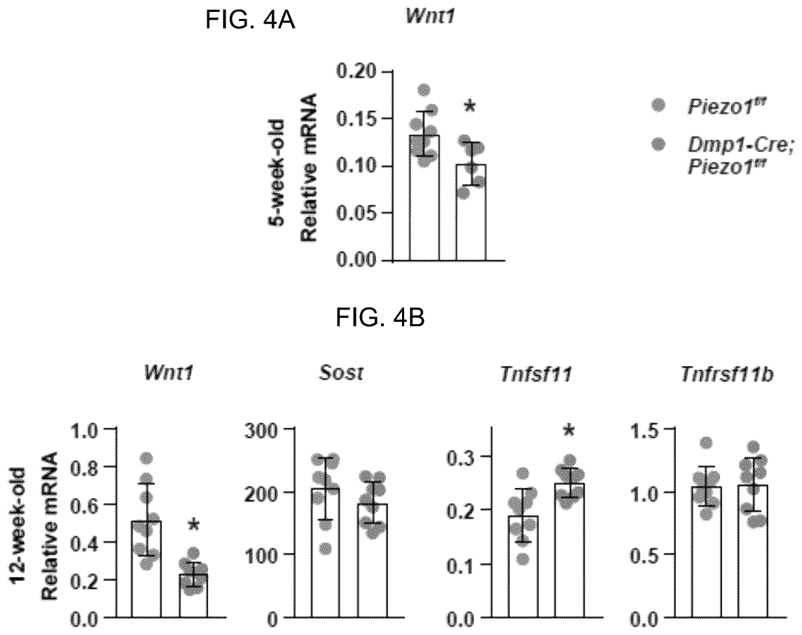

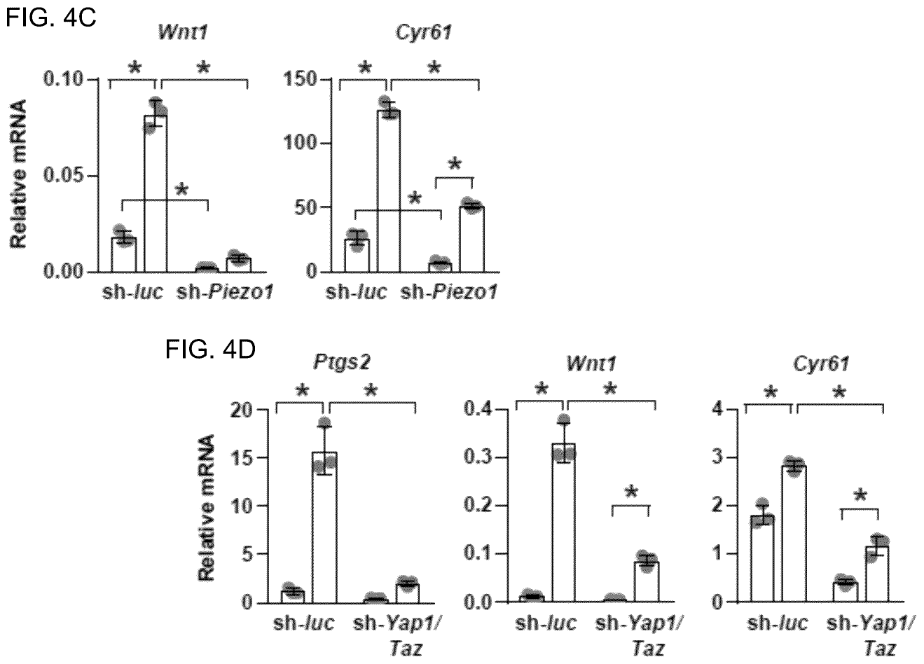

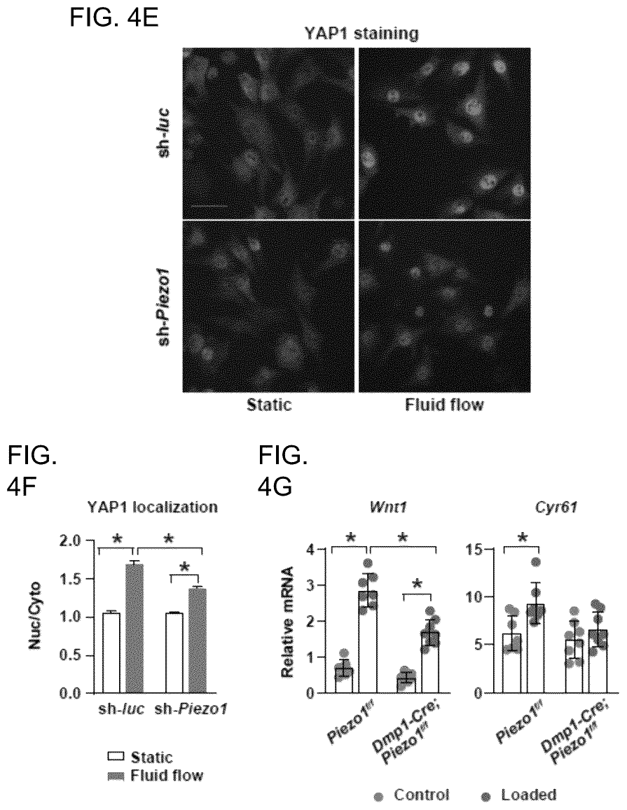

Piezo1 controls Wnt1 expression via YAP1 and TAZ. To understand the molecular mechanisms by which Piezo1 increases bone mass, we compared expression of genes known to influence bone formation and resorption between Dmp1-Cre; Piezo1^{f/f} mice and control littermates. Production of Wnt1 or the Wnt signaling inhibitor Sclerostin (Sost) by osteocytes represent critical stimulatory or inhibitory signals to bone formation, respectively (Luther et al., 2018, Li et al., 2008). Wnt1 mRNA was lower in cortical bone shafts of conditional knockout mice at both 5 and 12 weeks of age while the expression of Sost was unaffected (FIG. 4A, 4B). Consistent with increased osteoclast number, expression of the essential pro-osteoclastogenic cytokine RANKL (Tnfsf11) was higher in the conditional knockout mice (FIG. 4B). In contrast, expression of OPG (Tnfrsf11b), a secreted decoy receptor for RANKL, was not different between the genotypes (FIG. 4B), despite our observation of reduced Tnfrsf11b expression in MLO-Y4 cells lacking Piezo1 (FIG. 1E).

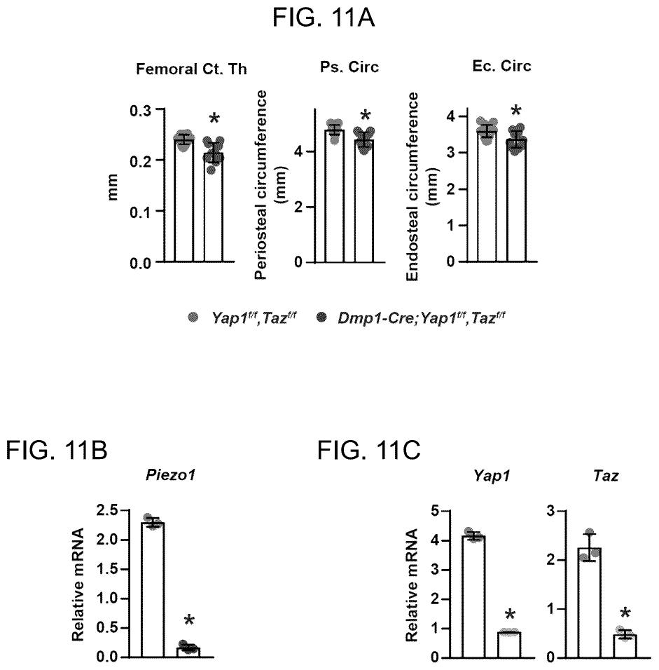

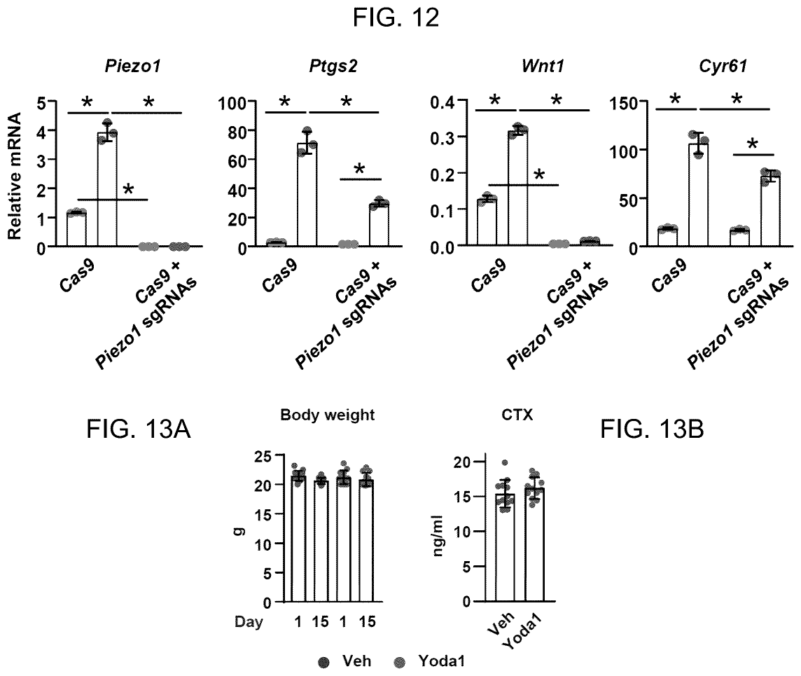

The expression of Wnt1 can be stimulated by mechanical loading in mice (Holguin et al., 2016). Therefore, we determined whether mechanical signals increase Wnt1 expression via Piezo1. Fluid shear stress increased Wnt1 expression in MLO-Y4 cells but this was blunted after knock-down of Piezo1 (FIG. 4C). Basal expression of Wnt1 was also reduced by Piezo1 knock-down (FIG. 4C). YAP1 and TAZ are two related transcriptional cofactors that can be activated by mechanical signals, including fluid flow and matrix rigidity, and recently Piezo1 has been shown to control their activity (Wang et al., 2016, Dupont et al., 2011, Pathak et al., 2014). We have shown previously that deletion of Yap1 and Taz using Dmp1-Cre decreases bone mass, due to both reduced bone formation and increased osteoclast number (Xiong et al., 2018). Here, we analyzed the diaphysis of femurs of these mice and found that cortical thickness, periosteal circumference, and endocortical circumference were significantly decreased in Dmp1-Cre; Yap1^{f/f}, Taz^{f/f} mice compared to their Yap1^{f/f}, Taz^{f/f} littermates (FIG. 11A). Because these changes were similar to the ones seen in cortical bone of Dmp1-Cre; Piezo1^{f/f} mice, we examined whether Piezo1 controls Wnt1 expression via YAP1 and TAZ. Silencing the Piezo1 gene in MLO-Y4 cells decreased the expression of Cyr61, a YAP1 and TAZ target gene, and blunted fluid shear stress induction of Cyr61 expression (FIG. 4C). We then silenced the Yap1 and Taz genes in MLO-Y4 cells to examine whether these factors are required for the stimulation of Wnt1 by fluid shear stress. We found that lack of Yap1 and Taz blunted the response to fluid flow including the increase in Ptgs2, Wnt1, and Cyr61 expression (FIG. 4D). Knock-down of Piezo1 and Yap1/Taz was confirmed by mRNA abundance (FIG. 11B, 11C). Importantly, silencing Piezo1 blunted YAP1 activation caused by fluid shear stress, indicated by blunted nuclear translocation of YAP1 (FIG. 4E, 4F). Similarly, we deleted Piezo1 in UAMS-32 cells, a murine osteoblastic cell line, using CRISPR/Cas9 and found that expression of Ptgs2, Wnt1, and Cyr61 induced by fluid flow were blunted in Piezo1 knock out cells (FIG. 12). To determine whether Piezo1 is required for Wnt1 expression induced by mechanical loading in vivo, we applied one bout of compressive loading on the tibia of Dmp1-Cre; Piezo1^{f/f} mice and their Piezo1^{f/f} littermates with +1200 με peak strain at the midshaft. Mechanical loading increased Wnt1 and Cyr61 expression in control mice (FIG. 4G). However, these increases were blunted in Dmp1-Cre; Piezo1^{f/f} mice (FIG. 4G). Taken together, these results indicate that stimulation of Piezo1 by mechanical signals increases Wnt1 expression at least in part via activation of YAP1 and TAZ.

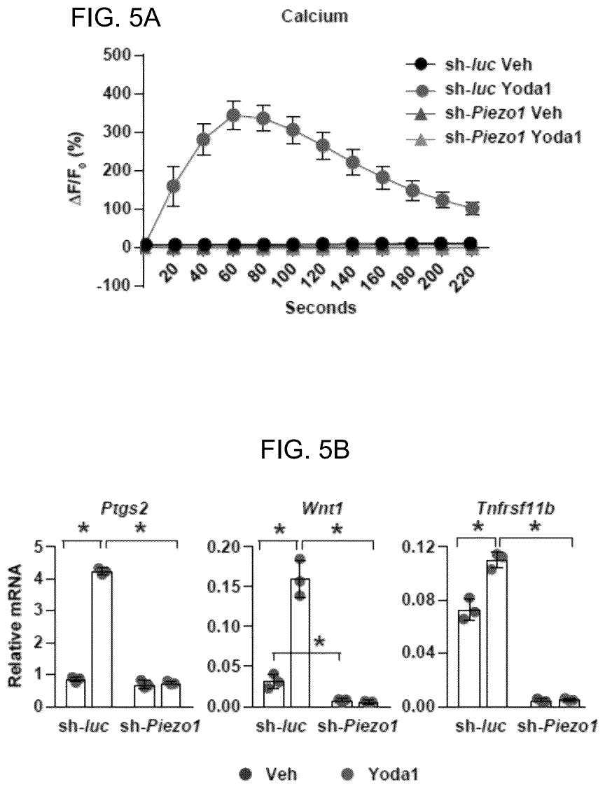

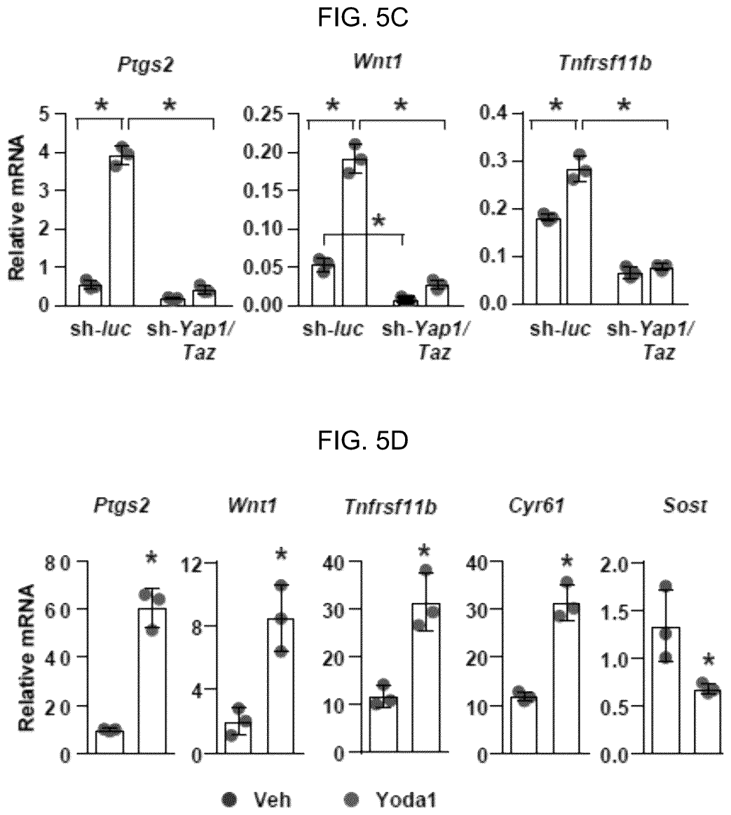

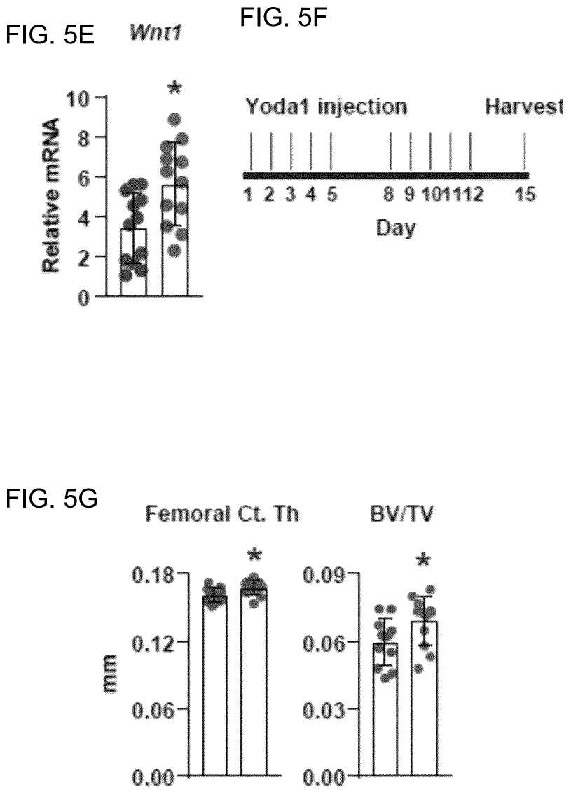

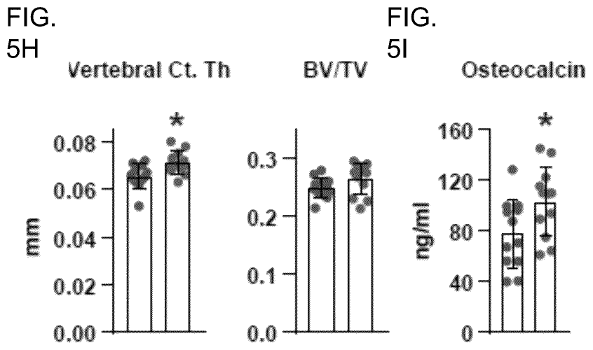

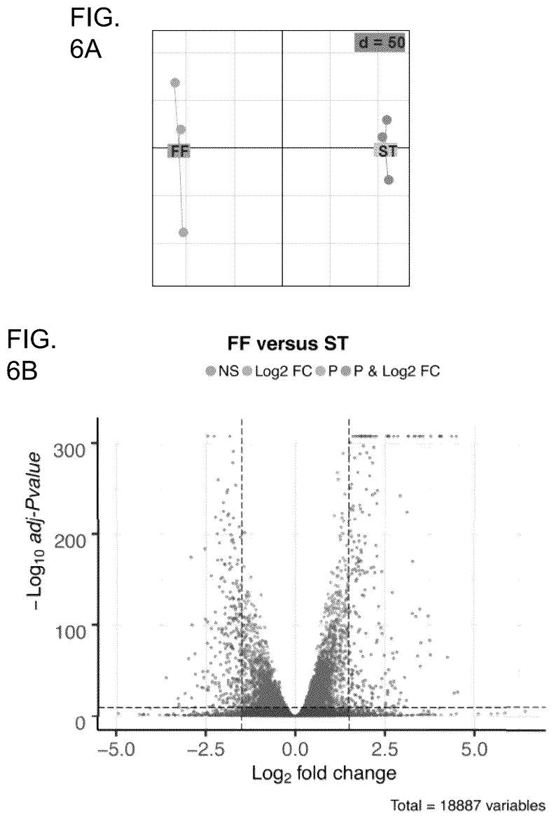

Activation of Piezo1 mimics the effects of mechanical stimulation on osteocytes. Finally, we determined whether activation of Piezo1 is sufficient to mimic the effects of mechanical stimulation in osteocytes and bone. Treatment of MLO-Y4 cells with Yoda1, a small molecule agonist of Piezo1 (Syeda et al., 2015), increased intracellular calcium concentration (FIG. 5A), and stimulated expression of Ptgs2, Wnt1, and Tnfrsf11b (FIG. 5B), similar to the effect of fluid flow on these cells. Importantly, silencing of Piezo1 completely prevented the increase of intracellular calcium (FIG. 5A), as well as the changes in gene expression induced by Yoda1 (FIG. 5B). Likewise, silencing Yap1 and Taz in MLO-Y4 cells significantly blunted the increase of Ptgs2, Wnt1, and Tnfrsf11b by Yoda1, indicating that the response to Yoda1 also requires YAP1 and TAZ (FIG. 5C). Yoda1 also promoted expression of Ptgs2, Wnt1, Tnfrsf11b, Cyr61, and decreased Sost in cortical bone organ cultures from C₅₇BL/6J mice (FIG. 5D). Importantly, Yoda1 increased Wnt1 expression in osteocyte-enriched cortical bone in vivo (FIG. 5E). These results demonstrated that Yoda1 mimics the response to fluid flow in authentic osteocytes.

To determine whether Yoda1 is able to increase bone mass in vivo, we administered Yoda1 to 4-month-old female WT C₅₇BL/6J mice for 2 weeks (FIG. 5F). Yoda1 did not alter body weight but increased cortical thickness and cancellous bone mass in the distal femur (FIG. 5G). Yoda1 also increased cortical thickness in the vertebra (FIG. 5H). However, we did not detect changes in cancellous bone volume in vertebrae (FIG. 5H). Consistent with the effect on bone mass, the serum levels of osteocalcin, a bone formation marker, were increased in Yoda1-treated mice (FIG. 5I). In contrast, we did not observe changes in the serum levels of CTX, a bone resorption marker (FIG. 13B). Our results demonstrate that activation of Piezo1 by Yoda1 mimics the effects of fluid shear stress on osteocytes and increases bone mass in mice.

### Discussion

Loss of function studies in epithelial cells have shown that Piezo1 responds to various forms of mechanical forces, including membrane stretch, static pressure, and fluid shear stress (Li et al., 2014, Gudipaty et al., 2017, Miyamoto et al., 2014). Moreover, Piezo1 can be activated by mechanical perturbations of the lipid bilayer alone, demonstrating its role in mechanosensation (Syeda et al., 2016). Here, the rapid response of MLO-Y4 cells to fluid shear stress is blunted by knocking-down Piezo1 indicating its important role in mechanosensation in bone cells. In addition, the basal skeletal phenotype of mice lacking Piezo1 in osteoblasts and osteocytes suggests that they have a reduced ability to respond to mechanical stimulation. Direct testing of this idea by performing an anabolic loading regime confirmed that the bones of the conditional knockout mice were less responsive to mechanical signals than controls. This decrease cannot be attributed to intrinsic cell defect since cell survive is not affected by Piezo1 deletion. Thus, our studies demonstrate that Piezo1 plays a critical role in sensing mechanical signals and maintaining bone homeostasis. In humans, truncation mutations in Piezo1 cause a recessive form of generalized lymphatic dysplasia but a musculoskeletal phenotype has not been reported (Fotiou et al., 2015). Nonetheless, SNPs in the human Piezo1 locus are associated with low bone mineral density and increased fracture risk (Morris et al., 2019).

We identified Wnt1 as a potential downstream effector of Piezo1. Previous studies have shown that mechanical loading increases Wnt1 expression in murine bone (Holguin et al., 2016, Kelly et al., 2016). Importantly, deletion of Wnt1 in osteoblasts and osteocytes using a Dmp1-Cre transgene produced a skeletal phenotype that resembles the one we observed by deletion of Piezo1 using the same Cre driver strain (Joeng et al., 2017). Taken together, these results suggest that mechanical signals stimulate Wnt1 expression via activation of Piezo1. Cell culture studies demonstrate that Piezo1 is required for YAP1 nuclear localization in neural stem cells (Pathak et al., 2014). Consistent with this, we found that Piezo1 controls nuclear translocation of YAP1 induced by fluid flow in MLO-Y4 cells. YAP1 and TAZ have been implicated as mediators of the response to mechanical signals in a variety of cell types (Dupont et al., 2011, Hansen et al., 2015). Our finding that YAP1 and TAZ are required for stimulation of Wnt1 by fluid flow or Yoda1 suggests that mechanical activation of Piezo1 stimulates Wnt1 expression in osteocytes, at least in part, by activating YAP1 and TAZ. Consistent with this idea, deletion of Yap1 and Taz in mature osteoblasts and osteocytes caused a skeletal phenotype that was similar to deletion of Piezo1, albeit less pronounced (Xiong et al., 2018). The milder bone phenotype of Yap1/Taz conditional knockout mice suggests that YAP1 and TAZ are not the only downstream effectors of Piezo1 in osteoblast lineage cells.

Similar to unloading, deletion of Piezo1 in osteoblasts and osteocytes led to not only decreases in bone formation, but also increases in RANKL expression and bone resorption. Indeed, increased RANKL expression as well as osteoclast number have been observed in hind-limb unloaded mice (Xiong et al., 2011). In our previous studies, we detected an increase in osteoclast number in mice that lack Yap1 and Taz in osteoblasts and osteocytes (Xiong et al., 2018), suggesting that YAP1 and TAZ are downstream effectors of Piezo1 in controlling osteoclast formation. Thus, loss of Piezo1 in osteoblasts and osteocytes mimics the overall effect of unloading on the skeleton, further supporting the idea that Piezo1 is a mechanosensor in bone.

Activation of Piezo1 using a Piezo1 agonist mimics the effects of fluid flow in various cell types including endothelial cells, erythrocytes, platelets, and smooth muscle cells (Cahalan et al., 2015, Li et al., 2014, Ilkan et al., 2017, Rode et al., 2017). In addition, Piezo1 agonist administration promotes lymphatic valve formation during development (Choi et al., 2019). Here, we showed that Piezo1 activation mimics the impact of mechanical stimulation in cultured osteocytic cells as well as ex vivo bone organ cultures. More importantly, administration of a Piezo1 agonist to mice increased bone mass and elevated a bone formation marker in the circulation, demonstrating that activation of Piezo1 is a potential target for anabolic bone therapy. It is important to note that bone anabolism requires only transient mechanical stimulation of the skeleton in rodents or humans (Vlachopoulos et al., 2018, Hinton et al., 2015, Kato et al., 2006). Therefore, it is possible that selectivity for bone anabolism may be achieved by administration regimes that result in only transient activation of Piezo1 by ligands such as Yoda1.

In summary, our studies demonstrate a critical role for Piezo1 in the maintenance of bone homeostasis and suggest that this occurs via mediation of mechanosensation in osteoblasts, osteocytes, or both. Our finding that activation of Piezo1 mimics the effects of mechanical stimulation on bone cells and increases bone mass in mice sets the stage for exploration of this pathway as a therapeutic target for osteoporosis.

### Materials and Methods

TABLE 1
Resources

| Reagent type | | | | |
|---|---|---|---|---|
| (species) or | | Source or | | Additional |
| resource | Designation | reference | Identifiers | information |
| Genetic | Mouse: | Jackson | JAX: 029213; | |
| reagent (M. | Piezo1^{f/f} | Laboratories | RRID: IMSR_ | |
| musculus) | (Piezo1^{tm2.1Apat}/J) | | JAX: 029213 | |
| Genetic | Mouse: | Bivi et al., | N/A | |
| reagent (M. | Dmp1-Cre | 2012 | | |
| musculus) | | | | |
| Genetic | Mouse: | Xin et al., | N/A | |
| reagent (M. | Yap1^{f/f}; Taz^{f/f} | 2013 | | |
| musculus) | | | | |
| Genetic | Mouse: WT | Jackson | JAX: 000664; | |
| reagent (M. | C57BL/6J | Laboratories | RRID: IMSR_ | |
| musculus) | | | JAX: 000664 | |
| commercial | Mouse | Thermo | Cat# J64239 | |
| assay or kit | Osteocalcin | Fisher | | |
| | Immunoassay | | | |
| | Kit | | | |
| commercial | Fluo-8 | Abcam | Cat# ab112129 | |
| assay or kit | Calcium Flux | | | |
| | Assay Kit | | | |
| commercial | RatLaps ™ | Immunodiag | Cat# AC-06F1 | |
| assay or kit | (CTX-I) EIA | nostic | | |
| | kit | Systems | | |
| commercial | TruSeq | Illumina | Cat# 20020594 | |
| assay or kit | stranded | | | |
| | mRNA kit | | | |
| commercial | High- | Life | Cat# 4368813 | |
| assay or kit | capacity | technologies | | |
| | CDNA | | | |
| | reverse | | | |
| | transcription | | | |
| | kit | | | |
| commercial | RNeasy mini | QIAGEN | Cat# 74106 | |
| assay or kit | kit | | | |
| cell line | 293T | ATCC | CRL-3216 | |
| (Murine) | | | | |
| cell line | MLO-Y4 | Kato et, al. | | |
| (Murine) | | 1997 | | |
| cell | UAMS-32 | O'Brien et, | | Cell line |
| line(Murine) | | al. 1999 | | maintained in |
| | | | | Charles |
| | | | | O'Brien lab |
| transfected | Piezo1 shRNA | Zhang, et, al | Oligo | CCGGTCGG |
| construct (M. | forward | 2017 | | CGCTTGCT |
| musculus) | | | | AGAACTTC |
| | | | | ACTCGAGT |
| | | | | GAAGTTCT |
| | | | | AGCAAGCG |
| | | | | CCGATTTTT |
| | | | | G (SEQ ID |
| | | | | NO: 1) |
| transfected | Piezo1 shRNA | Zhang, et, al | Oligo | AATTCAAA |
| construct (M. | reverse | 2017 | | AATCGGCG |
| musculus) | | | | CTTGCTAG |
| | | | | AACTTCAC |
| | | | | TCGAGTGA |
| | | | | AGTTCTAG |
| | | | | CAAGCGCC |
| | | | | GA (SEQ ID |
| | | | | NO: 2) |
| transfected | Yap1 shRNA | Sigma- | TRCN0000238432 | |
| construct (M. | | Aldrich | | |
| musculus) | | | | |
| transfected | Taz shRNA | Sigma- | TRCN0000095951 | |
| construct (M. | | Aldrich | | |
| musculus) | | | | |
| sequenced- | Piezo1 | Life | Mm01241549_ | |
| based reagent | | technologies | m1 | |
| sequenced- | Piezo2 | Life | Mm01265861_ | |
| based reagent | | technologies | m1 | |
| sequenced- | Ptgs2 | Life | Mm00478374_ | |
| based reagent | | technologies | m1 | |
| sequenced- | Cyr61 | Life | Mm00487498_ | |
| based reagent | | technologies | m1 | |
| sequenced- | Wnt1 | Life | Mm01300555_ | |
| based reagent | | technologies | g1 | |
| sequenced- | Yap1 | Life | Mm01143263_ | |
| based reagent | | technologies | m1 | |
| sequence- | Taz | Life | Mm01289583_ | |
| based reagent | | technologies | m1 | |
| sequence- | Tnfsf11 | Life | Mm00441906_ | |
| based reagent | | technologies | m1 | |
| sequence- | Tnfrsf11b | Life | Mm00435452_ | |
| based reagent | | technologies | m1 | |
| sequence- | Sost | Life | Mm00470479_ | |
| based reagent | | technologies | m1 | |
| sequence- | Mrps2 | Life | Mm00475529_ | |
| based reagent | | technologies | m1 | |
| sequence- | Piezo1 | This paper | | GGTTATTC |
| based reagent | sgRNA | | | CTGTGAGG |
| | | | | CCCG (SEQ |
| | | | | ID NO: 3) |
| sequence- | Piezo1 | This paper | | TTAGGATT |
| based reagent | sgRNA | | | CGGCTCAC |
| | | | | AGAG (SEQ |
| | | | | ID NO: 4) |
| chemical | Yoda1 | Sigma- | Cat# SML1558 | |
| compound, | | Aldrich | | |
| drug | | | | |
| chemical | Puromycin | Sigma- | Cat# P8833 | |
| compound, | dihydrochloride | Aldrich | | |
| drug | | | | |
| chemical | G418 | Sigma- | Cat# G8168 | |
| compound, | disulfate | Aldrich | | |
| drug | | | | |
| Antibody | YAP1 | Cell | Cat# 14074S; | 1:200 |
| | | signaling | RRID: AB_2650491 | |
| Antibody | Goat anti- | Abcam | Cat# ab150077; | 1:200 |
| | Rabbit IgG | | RRID: AB_2630356 | |
| | (Alexa Fluor ® | | | |
| | 488) | | | |
| software, | Prism 8 | GraphPad | https://www. | |
| algorithm | | | graphpad.com/ | |
| | | | scientific- | |
| | | | software/prism/ | |
| software, | ImageJ | NIH | https://imagej. | |
| algorithm | | | nih.gov/ij | |

Mice. The generation of mice harboring Piezo1 conditional allele, termed Piezo1^{f/f} mice, was described previously (Cahalan et al., 2015). Mice harboring both Yap1 and Taz conditional alleles, termed Yap1^{f/f}; Taz^{f/f} mice were kindly provided by Eric N. Olson (UT Southwestern Medical Center, Texas) and were described previously (Xin et al., 2013). The 8 kb Dmp1-Cre transgenic mice were described previously (Bivi et al., 2012). To generate Dmp1-Cre; Piezo1^{f/f} mice and littermates, we mated Piezo1^{f/f} mice (crossed into C₅₇BL/6J for more than 10 generations) and Dmp1-Cre mice (crossed into C₅₇BL/6J for more than 10 generations). Dmp1-Cre; Yap1^{f/f}, Taz^{f/f} mice and littermates were obtained by mating Yap1^{f/f}, Taz^{f/f} mice (mixture of 129/Sv and C₅₇BL/6J) and Dmp1-Cre mice (crossed into C₅₇BL/6J for more than 10 generations). We housed all mice in the animal facility of the University of Arkansas for Medical Sciences. Animal studies were performed in strict accordance with the recommendations in the Guide for the Care and Use of Laboratory Animals of the National Institutes of Health. Animal use protocols (3782, 3805, and 3897) were approved by the Institutional Animal Care and Use Committee (IACUC) of the University of Arkansas for Medical Sciences. All of the animals were handled according to approved protocols.

To quantify cancellous bone formation, we injected mice with calcein (20 mg/kg body weight) intraperitoneally 7 and 3 days before harvest. To quantify periosteal and endocortical bone formation, we injected mice with calcein (20 mg/kg body weight) and Alizarin Red (20 mg/kg body weight) 10 and 3 days before harvest. For gene expression, we injected Yoda1 into 4-month-old female C₅₇BL/6J mice one time and harvested tibiae 4 hours later for RNA extraction. For bone mass evaluation, we injected Yoda1 into 4-month-old female C₅₇BL/6J mice 5 consecutive days per week for 2 weeks (day 1-5 and day 8-12) and harvested the mice at day 15 for analysis. Yoda1 (Sigma, St. Louis, MO) was dissolved in DMSO at 40 mM as a stock, diluted in 5% ethanol, and injected intraperitoneally at 5 μmol/kg body weight. Mice were rank-ordered by body weight and then assigned to Veh or Yoda1 groups to give identical group means. All investigators involved in data collection were blinded as to the genotype and group of the mice.

Cell line. HEK 293T cells were authenticated by ATCC. MLO-Y4 cells were created and authenticated in Dr. Lynda Bonewald's lab (Kato et al., 1997). We tested the MLO-Y4 cells by morphology and osteocytic gene expression such as RANKL and OPG. UAMS-32 cells were created and authenticated by Dr. Charles O'Brien (O'Brien et al., 1999, Fu et al., 2002). Cells were treated with plasmocin to prevent potential mycoplasma contamination.

Cell cultures. MLO-Y4 cells were cultured in α-MEM supplemented with 5% FBS, 5% BCS, and 1% penicillin/streptomycin/glutamine. Fifteen dynes/cm² oscillatory fluid shear stress was applied on MLO-Y4 cells at 1 Hz for 2 hours using an IBDI pump system (IBIDI, German). For Yoda1 treatment, cells were cultured in the presence of 10 μM Yoda1 (Sigma, St. Louis, MO) or DMSO for 2 hours. Immediately after the treatments, we isolated RNA from cells using RNeasy mini kit (Qiagen, German) for qPCR or RNA-seq analysis. To silence Piezo1, we generated Piezo1 shRNA expression plasmid using the following oligonucleotides in the pLKO.1-TRC cloning vector (Addgene Plasmid #10878, a gift from David Root): forward oligo: 5′-CCGGTC-GGCGCTTGCTAGAACTTCACTCGAGTGAAGTTCTAGCAAGCGCCGATTTTTG-3′ (SEQ ID NO: 1); reverse oligo: 5′-AATTCAAAAATCGGCGCTTGCTAGAACTTCACTCGAGTGAAGTTCTAGCAAGCGC-CGA-3′ (SEQ ID NO: 2) (Zhang et al., 2017). Yap1 shRNA (TRCN0000238432) and Taz shRNA (TRCN0000095951) were purchased from Sigma (St. Louis, MO). A shRNA against firefly luciferase was used as a control (Sigma, St. Louis, MO). For virus production, HEK293T cells were cultured in a 6-well culture plate and co-transfected with a total 3 μg of lentiviral shRNA vector, pMD2G (Addgene plasmid #12259, a gift from Didier Trono), and psPAX2 (Addgene plasmid #12260, a gift from Didier Trono) at the ratio of 2:0.9:0.4 using TransIT-LT1 transfection reagent (Mirus, Madison, WI). Culture media was changed 12 hours after transfection and viral supernatants were collected 48 hours after media change. Viral supernatants were filtered through a 0.45 μm filter and used immediately to transduce cells cultured in a 10 cm dish. Cells were then subjected to selection with G418 (100 μg/ml) or puromycin (25 μg/ml) for 5 days before treatment. To overexpress Piezo1 in MLO-Y4 cells, we transfected mPiezo1-IRES-eGFP (Addgene plasmid #80925, a gift from Ardem Patapoutian) into MLO-Y4 cells using TransIT-LT1 transfection reagent (Mirus, Madison, WI) and then treated these cells with 15 dynes/cm² oscillatory fluid shear stress at 1 Hz for 2 hours. Plasmids for expression of Cas9 and sgRNAs for knocking out Piezo1 in UAMS-32 cells were prepared by inserting oligonucleotides encoding the desired sgRNA sequence into the pX458 vector using the protocol recommended by the Zhang laboratory (Cong et al., 2013). Plasmids expressing Cas9 and Piezo1 sgRNAs were transfected into UAMS-32 cells using TransIT-LT1 transfection reagent (Mirus, Madison, WI). Cells were sorted into 96-well plates for single cell cloning 48 hours after transfection. Single cell colonies were then screened for homozygous deletion using the following primers: Forward: 5′-GCTGTCAGGGTAAGCAGTATC-3′ (SEQ ID NO: 5), Reverse: 5′-GGAATATGAGGACAGCAGTCC-3′ (SEQ ID NO: 6). All homozygous mutant cell colonies were then pooled together for further analysis. Cas9 transfected cells were used as a control. All in vitro cell culture experiments were performed three times with three technical replicates.

Femoral organ culture. Female mice at 5 weeks of age were euthanized in a CO₂ chamber. Femurs were dissected and both ends were removed in a culture hood. Bone marrow was then flushed out using PBS and the periosteal surface was scraped to remove periosteal cells. Femoral shafts were then cultured in a 12-well-plate with 1 ml of α-MEM supplemented with 10% FBS and 1% penicillin/streptomycin/glutamine for 24 hours. We then treated femur shafts with 10 μM Yoda1 (Sigma, St. Louis, MO) or DMSO for 4 hours. Femur shafts were then collected for RNA isolation and qPCR analysis. Ex vivo femoral organ culture was repeated twice with three biological replicates.

In vitro osteoblast differentiation. Bone marrow stromal cells were flushed out from long bones, collected into a 50 ml cubical tube, and filtered through a 40 μm cell strainer to obtain a single cell suspension. Bone marrow stromal cells were then seeded into a 12-well-plate at 5×10⁶ cells/well and cultured in α-MEM containing 10% fetal bovine serum, 1% penicillin/streptomycin/glutamine, 1% ascorbic acid, and 10 mM β-glycerolphosphate. Culture medium was changed every 3 days. After 21 days, the cultures were fixed with 10% buffered formalin and stained with an aqueous solution of 40 mM Alizarin Red to evaluate osteoblastogenesis.

RNA-seq analysis. Purified RNA was used as input for sequencing library preparation and indexing using the TruSeq stranded mRNA kit (Illumina, CA), following the manufacturer's protocol. The libraries were then pooled and sequenced using a NextSeq sequencer with 75 cycles of sequencing reaction. Data handling and processing were performed on the basis of a previous bioinformatics pipeline (Nookaew et al., 2012). The high-quality reads (phred quality score, >25; length after trimming, >20 bases) were obtained with the dynamic trimming algorithm in the SolexaQA++ toolkits (Cox et al., 2010), and aligned with the mouse genome version GRCm38 using BWA software (Li and Durbin, 2009). Then the alignment files (.bam) were used to generate read counts for statistical analysis. The differential gene expression analysis was performed using negative binomial based statistic (Love et al., 2014). The adjusted p-values were used for gene enrichment analysis based on Gene Ontology using the piano package (Varemo et al., 2013). Raw RNA-seq results have been deposited in GEO database under BioProject PRJNA551282 with accession numbers: SRR9598498, SRR9598497, SRR9598496, SRR9598495, SRR9598494, and SRR9598493.

Calcium concentration measurement. For intracellular calcium concentration measurement under fluid flow condition, 1×10⁵ MLO-Y4 cells were seeded in a μ-Slide I Luer (0.4 mm) fluid chamber slide (MIDI, Germany) overnight. One hour before initiating fluid flow, the culture medium was removed and 100 μl Hank's Buffer with Hepes (HHBS) containing 4μM Fluo-8 (Abcam, Cambridge, MA) was added to the culture, as described by the manufacturer. The cells were then cultured at 37° C. for 30 minutes. After additional incubation at room temperature for 30 minutes, the chamber slide was placed under a confocal microscope in order to record the intensity of fluorescence of MLO-Y4 cells. Fluorescence was recorded for 3 minutes before starting fluid flow using HHBS and then recorded for another 10 minutes. The increase of intracellular concentration was calculated by subtracting the initial mean fluorescence. For measuring intracellular calcium concentration in cells with Yoda1 treatment, we cultured 4,000 MLO-Y4 cells per well in a 96-well-plate. We preloaded the cells with Fluo-8 as described by the manufacturer and read the intensity of the fluorescence using a Victor X3 multi-label plate reader (Perkin Elmer, Waltham, MA) immediately after the treatment. We measured the fluorescence for 5 minutes at an interval of 20 seconds. The percentage of increase in intracellular calcium concentration was calculated as (Fₓ-F₀)/F₀.

Skeletal analysis. Tibial X-rays were obtained using an UltraFocus X-ray machine (Faxitron Bioptics, Tucson, Arizona) and BMD of the lumbar spine and femur were measured by dual-energy X-ray absorptiometry using a PIXImus Densitometer (GE-Lunar Corp.) Three dimensional bone volume and architecture of L4 vertebra, femur, and tibia were measured by μCT (model μCT40, Scanco Medical, Wayne, PA). The femur, vertebrae (L4), or tibia, were cleaned of soft tissues and fixed in 10% Millonig's formalin for 24 hours. Bone were then gradually dehydrated into 100% ethanol. Bone samples were loaded into a 12.3 mm diameter scanning tube and images acquired in the μCT40. The scans were integrated into 3D voxel images (1024×1024 pixel matrices for each individual planar stack) and a Gaussian filter (sigma=0.8, support=1) was used to reduce signal noise. Scanco Eval Program v.6.0 was used for measuring bone volume. A threshold of 220 mg/cm³ was applied to all scans at medium resolution (E=55 kVp, I=145 μA, integration time=200 ms) for trabecular bone measurements. The cortical bone and the primary spongiosa were manually excluded from the analysis. Trabecular bone measurements at the distal femur were made on 151 slices beginning 8-10 slices away from the growth plate and proceeding proximally. Trabecular bone measurements in the vertebra was determined using 100 slices (1.2 mm) of the anterior (ventral) vertebral body immediately inferior (caudal) to the superior (cranial) growth plate. All trabecular measurements were made by drawing contours every 10 to 20 slices and voxel counting was used for bone volume per tissue volume and sphere filling distance transformation indices, without pre-assumptions about the bone shape as a rod or plate for trabecular microarchitecture. Femoral cortical thickness, periosteal circumference, and endocortical circumference were measured at the mid-diaphysis. For tibial cortical thickness, we analyzed 18 slices 5 mm proximal from the distal tibiofibular junction. Vertebral cortical bone thickness was determined on the ventral cortical wall using contours of cross-sectional images, drawn to exclude trabecular bone. Cortical analysis were measured at a threshold of 260 mg/cm³. Calibration and quality control were performed weekly using five density standards and spatial resolution was verified monthly using a tungsten wire rod. We based beam-hardening correction on the calibration records. Corrections for 200 mg hydroxyapatite were made for all energies.

Histology. Lumbar vertebrae were fixed for 24 hours in 10% Millonig's formalin, dehydrated into 100% ethanol, embedded in methyl methacrylate, and then 5 μm longitudinal sections were obtained. After removal of plastic and rehydration, we stained sections for TRAP activity and counter-stained with T-blue. Quantitative histomorphometry was performed to determine osteoblast and osteoclast number using Osteomeasure system (OsteoMetrics, Decatur, GA) interfaced to an Axio image M2 (Carl Zeiss, NY). Bone formation rate was measured using unstained sections in Osteomeasure system. We used terminology recommended by the Histomorphometry Nomenclature Committee of the American Society for Bone and Mineral Research (Dempster et al., 2013). For quantification of periosteal and endocortical bone formation, femurs or tibiae were fixed in 10% Millonig's formalin for 24 hours, dehydrated into 100% ethanol, embedded in methyl methacrylate, and then 80 μm cross sections were obtained at the femoral mid-diaphysis for femoral sections and 5 mm proximal from the distal tibiofibular junction for tibial sections. We then measured mineralizing surface and mineral apposition rate using the Osteomeasure system.

Tibia axial loading. A cyclic axial load was applied to left tibia of mice to achieve +1200 με peak strain at the tibial midshaft using an Electroforce TA 5500 (TA Instruments, New Castle, DE). To determine the required load to achieve +1200 με peak strain for each genotype of experimental mice, axial loading was applied to harvested tibiae ex vivo. A single-element strain gauge (C₂A-06-015LW-120, VPG Micro-Measurements, Wendell, NC) was attached to the antero-medial surface of the tibia located 5 mm proximal from the distal tibiofibular junction using M-Bond 200 adhesive kit (VPG Micro-Measurements). We recorded the force-strain regressions using Electroforce TA 5500 software. We then applied the same amount of load to mice in vivo according to their genotype (8.5 Newton for Piezo1^{f/f} mice and 7.5 Newton for Dmp1-Cre; Piezo1^{f/f} mice). The left tibia of each mouse was loaded for 5 consecutive days per week for 2 weeks (day 1-5 and day 8-12), and the load was applied in 1200 cycles with 4-Hz triangle waveform and 0.1 s rest time between each cycle, a protocol shown to be anabolic (Sun et al., 2018). We injected calcein (Sigma, St. Louis, MO) and Alizarin Red (Sigma) intraperitoneally into mice 10 days and 3 days before euthanasia to label new bone formation. We euthanized the mice and collected tissues at day 15 for skeletal analysis. For gene expression analysis, we loaded left tibia of 4-month-old female mice with one bout of loading and harvested tibiae 5 hours after loading for RNA extraction.

Biomechanical testing. We performed three-point bending test on femurs at room temperature using a miniature bending apparatus with the posterior femoral surface lying on lower supports (7 mm apart) and the left support immediately proximal to the distal condyles. Load was applied to the anterior femoral surface by an actuator midway between the two supports moving at a constant rate of 3 mm/min to produce a physiological in vivo strain rate of 1% for the average murine femur. Maximum load (N) and displacement (mm) were recorded. The external measurements (length, width and thickness) of the femora were recorded with a digital caliper. We measured the moment of inertia in the midshaft of femur using μCT (model μ40, Scanco Medical). The mechanical properties were normalized for bone size and ultimate strength and stress (N/mm²; in megapascals and MPa) was calculated.

Quantitative PCR. Organs and whole bones were harvested from animals, removed of soft tissues, and stored immediately in liquid nitrogen. We prepared osteocyte-enriched bone by removing the ends of femurs and tibias and then flushing the bone marrow with PBS. We then scraped the bone surface with a scalpel and froze them in liquid nitrogen for later RNA isolation, or decalcified them for genomic DNA isolation. We isolated total RNA using TRIzol (Life technologies, NY), according to the manufacturer's instructions and prepared cDNA using High Capacity first strand cDNA synthesis kit (Life Technologies). We performed quantitative RT-PCR using the following Taqman assays from Applied Biosystems: Piezo1 (Mm01241549_m1); Piezo2 (Mm01265861_m1); Ptgs2 (Mm00478374_m1); Cyr61 (Mm00487498_m1); Wnt1 (Mm01300-555_g1); Yap1 (Mm011432-63_m1); Taz (Mm01289583_m1); Tnfsf11 Mm00441906_m1; Tnfrsf11b (Mm00435452_m1); Sost (Mm00470479_m1); and ribosomal protein S2 (Mrps2) (Mm00475529_m1). We calculated relative mRNA amounts using the ΔCt method (Livak and Schmittgen, 2001). We isolated genomic DNA from decalcified cortical bone after digestion with proteinase K and phenol/chloroform extraction. We obtained two custom Taqman assays from Applied Biosystems for quantifying Piezo1 gene deletion efficiency: one specific for sequences between the loxP sites and the other specific for sequences downstream from the 3′ loxP site.

Immunostaining. Cultured cells were fixed in 4% freshly prepared paraformaldehyde for 15 minutes. Slides were washed in PBST for 5 minutes, pretreated with PBS containing 0.1% Triton X-100 for 20 minutes, and blocked in 2.5% normal goat serum for one hour. Anti-YAP1 antibody (14074S, Cell signaling, Danvers, MA) was diluted 1:200 in PBST containing 2.5% normal goat serum and incubated with the chamber slides overnight at 4° C. followed by rinsing and additional incubation for 1 hour with goat anti-rabbit IgG H&L(1:200) (Alexa Fluor® 488) (ab150077, Abcam, Cambridge, MA). Non-immune goat IgG was used as a negative control. Slides were mounted with aqueous mounting medium (H-1000, VECTOR LABORATORIES, INC., Burlingame, CA). Stained slides were imaged using Axio imager M2 fluorescence microscope (Carl Zeiss, NY). Mean fluorescence intensity was quantified using ImageJ (NIH, Bethesda, Maryland).

Osteocalcin and CTX ELISA. Circulating osteocalcin and CTX in serum was measured using a mouse Osteocalcin enzyme immunoassay kit (Thermo Fisher) and RatLaps™ (CTX-I) EIA kit (Immunodiagnostic Systems, Boldon, UK) respectively according to the manual provided by manufacturers. Blood was collected by retro-orbital bleeding into 1.7 mL microcentrifuge tubes. Blood was then kept at room temperature for one hour and centrifuged at 1500×g for 10 minutes to separate serum from cells.

Statistical analysis. GraphPad Prism 7 software (GraphPad, San Diego) was used for statistical analysis. Two-way analysis of variance (ANOVA) or Student's t-test were used to detect statistically significant treatment effects, after determining that the data were normally distributed and exhibited equivalent variances. All t-tests were two-sided. P-values less than 0.05 were considered as significant. Error bars in all figures represent s.d.

### Piezo1 Agonists are Potent Stimulators of Piezo1

The compounds disclosed herein are potent stimulators of Piezo1. Piezo1 agonists described herein were prepared and tested as stimulators of Piezo1.

To identify Piezo1 stimulators, we treated MLO-Y4 cells with compounds Yoda1, YW-3-159, W-3-163, W-3-168-1, W-3-168-2, W-3-170, W-3-173-1, W-3-173-2, and YW-3-174. Gene expression of Wnt1, Ptgs2, and Tnfrsf11b was measured to determine the activation of Piezo1. In addition, intracellular calcium concentration was measured to determine the activity of Piezo1 channel. MLO-Y4 cells lacking Piezo1 channel were also treated with the compounds to demonstrate the specificity of the compounds. Yoda1 was used as a positive control in all experiments.

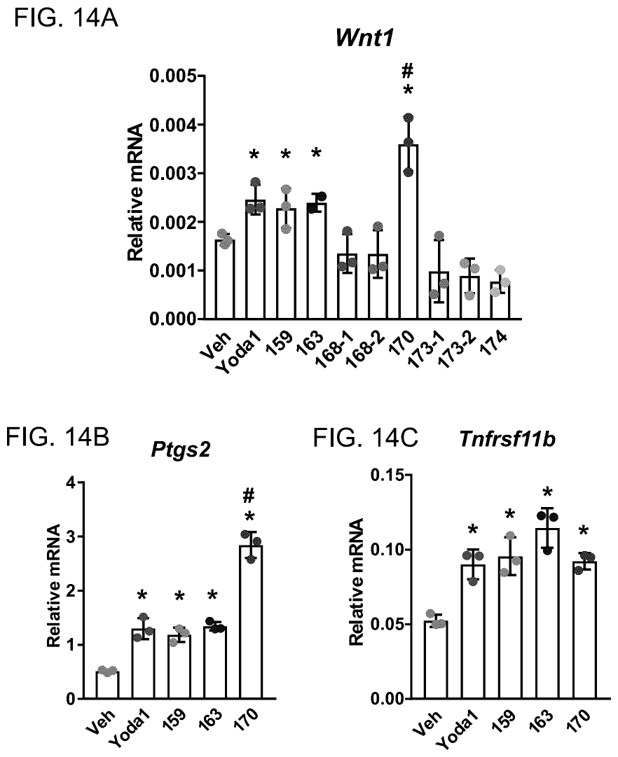

Each of Yoda1, W-3-159, W-3-163, and W-3-170 induced higher Wnt1 expression in MLO-Y4 cells treated than vehicle (Veh) (FIG. 14A). W-3-170 also induced higher Wnt1 expression than the positive control, Yoda1 (FIG. 14A).

Each of Yoda1, W-3-159, YW-3-163, and W-3-170 induced higher Ptgs2 expression in MLO-Y4 cells treated than vehicle (Veh) (FIG. 14B). W-3-170 also induced higher Ptgs2 expression than the positive control, Yoda1 (FIG. 14B).

Each of Yoda1, W-3-159, W-3-163, and W-3-170 induced higher Tnfrsf11b expression in MLO-Y4 cells treated than vehicle (Veh) (FIG. 14C).

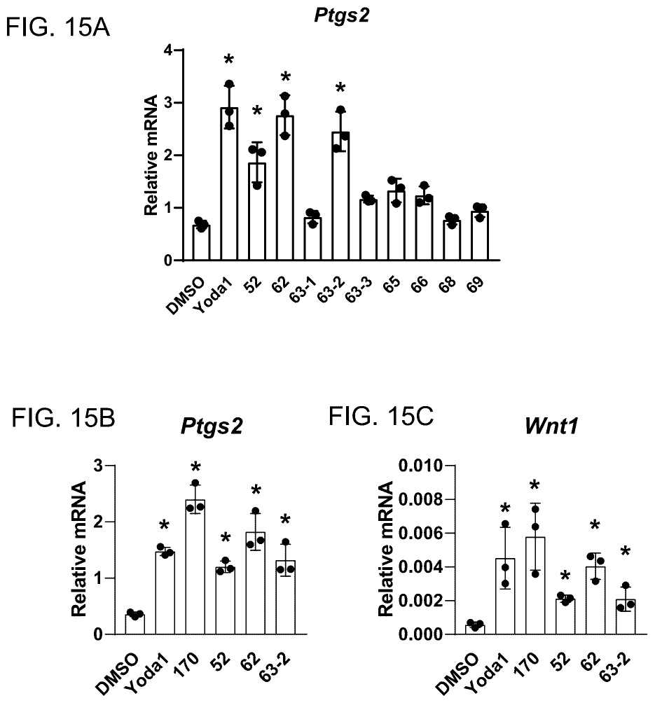

Additional compounds W-4-52, W-4-62, W-4-63-1, W-4-63-2, W-4-63-3, YW-4-66, W-4-68, and W-4-92 were also tested. W-4-62 and W-4-63-2 promoted expression of Ptgs2 and Wnt1 in MLO-Y4 cells (FIG. 15A-15C). YW-3-170 and Yoda1 were used as positive controls.

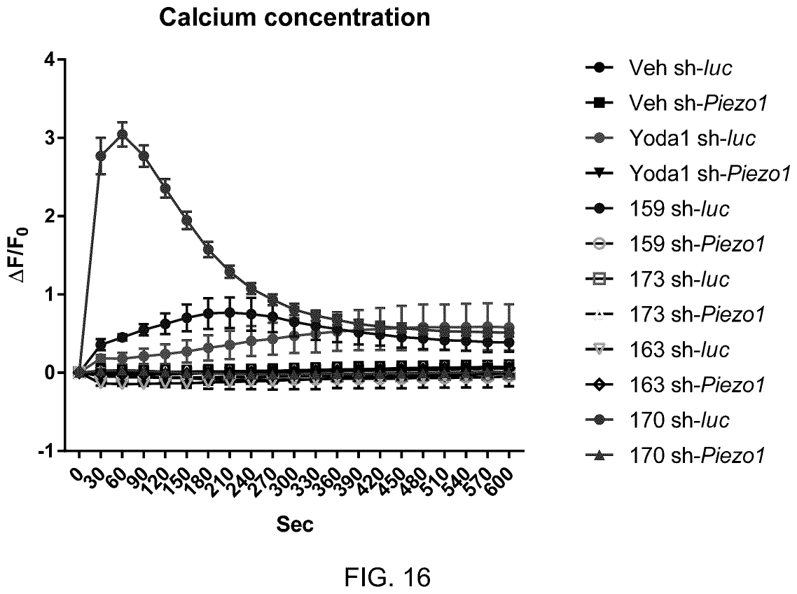

We then compared the intracellular calcium concentration induced by YW-3-159, YW-3-163, YW-3-170, and Yoda1 in MLO-Y4 cells and found that YW-3-170 induced much more calcium influx than Yoda1 (FIG. 16). More importantly, knockdown of Piezo1 completely blocked the calcium influx induced by these compounds, indicting their specificity (FIG. 16).

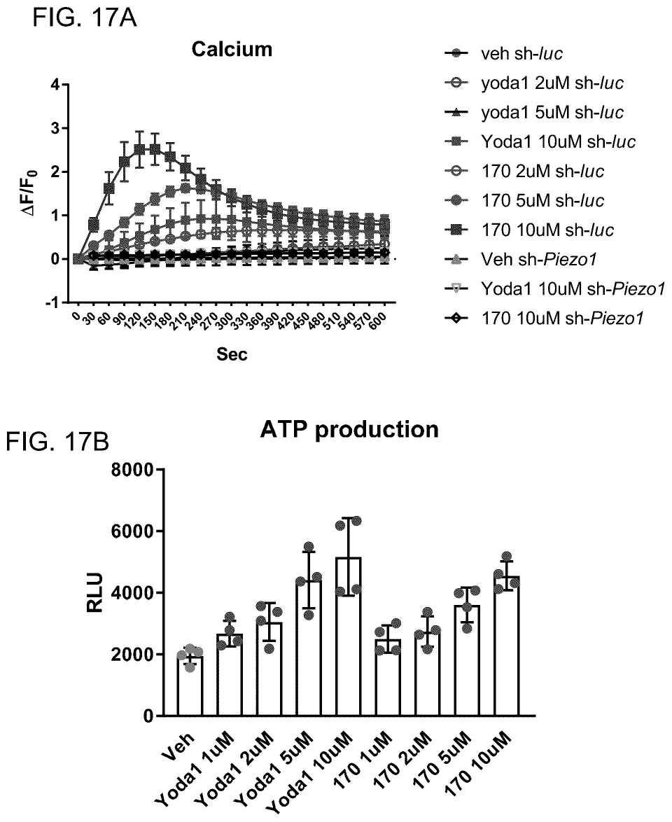

We next examined the calcium influx induced by YW-3-170 and Yoda1 at different doses. Our results showed that YW-3-170 increases intracellular calcium concentration dose-dependently and induces higher intracellular calcium concentration than Yoda1 at 2 μM, 5 μM, and 10 μM (FIG. 17A). Moreover, both YW-3-170 and Yoda1 increased ATP production in MLO-Y4 cells in a dose-dependent manner (FIG. 17B), similar to the effect of fluid shear stress. Taken together, these results demonstrated that YW-3-159, YW-3-163, YW-3-170, YW-4-62, and YW-4-63-2 are potent agonists for Piezo1.

We have identified several novel Piezo1 stimulators including YW-3-159, YW-3-163, YW-3-170, YW-4-62, and YW-4-63-2. These compounds exhibited comparable or superior activity in activating Piezo1 in MLO-Y4 cells vitro compared to Yoda1, which has been shown in mice to promote bone formation in this application. The specificity of these compounds to activate Piezo1 was demonstrated by the loss of their ability to stimulate calcium influx in Piezo1 -deficient cells. This is consistent with our findings that Yoda1 induced changes in gene expression of Wnt1, Ptgs2, and Tnfrsf11b requires Piezo1. Therefore, YW-3-159, YW-3-163, YW-3-170, YW-4-62, and YW-4-63-2 are potent and specific Piezo1 agonists.

### Comparative Example of Yoda1 and YW-3-170

Mitochondria are critical components of cells and their function has been associated with osteoblastogenesis and bone homeostasis i. In humans, mitochondrial point mutation m.3243A>G leads to mitochondrial dysfunction and is associated with decreased bone mass and strength. Increased mitochondrial function is also associated with mechanical stimulation. Therefore, we sought to determine whether activation of Piezo1 by Yoda1 or YW-3-170 increases mitochondria function in bone cells.

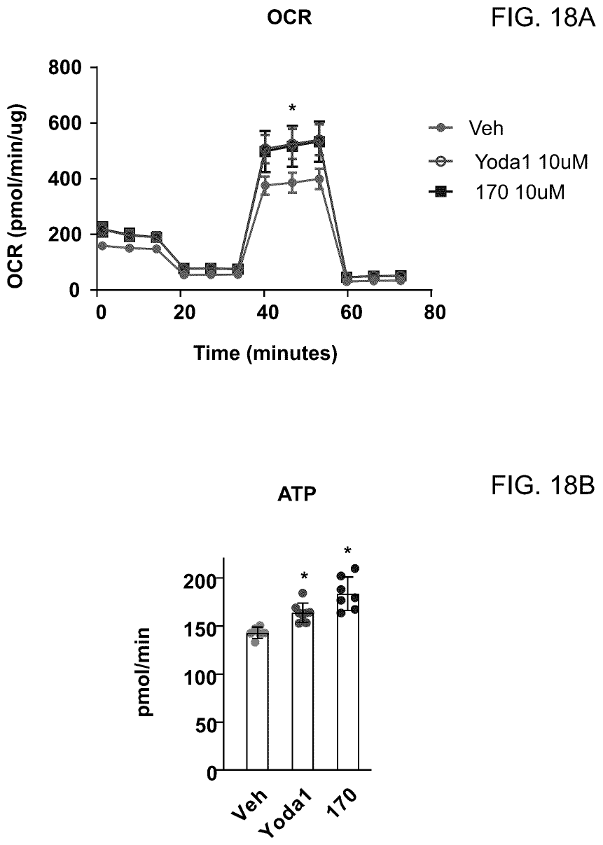

To determine whether activation of Piezo1 by Yoda1 or YW-3-170 is associated with increased mitochondrial function, we measured oxygen consumption rate and ATP production in calvaria cells treated by Yoda1 or YW-3-170. Wildtype and Piezo1 knockout calvarial cells were cultured in α-MEM medium supplemented with 10% fetal bovine serum and treated with 10 μM Yoda1 or YW-3-170 for 1 hour. Oxygen consumption rate and ATP production were measured using seahorse. In wildtype calvaria cells, we found that both Yoda1 and YW-3-170 increased mitochondrial oxygen consumption rate at basal level as well as stimulated level (FIGS. 18A-18B). However, these changes were significantly blunted in calvaria cells lacking Piezo1. Consistent with these results, ATP production was stimulated by Yoda1 in wildtype cells. Moreover, YW-3-170 stimulated more ATP production than Yoda1 at 10 μM. Importantly, these increases were diminished in Piezo1 knockout cells.

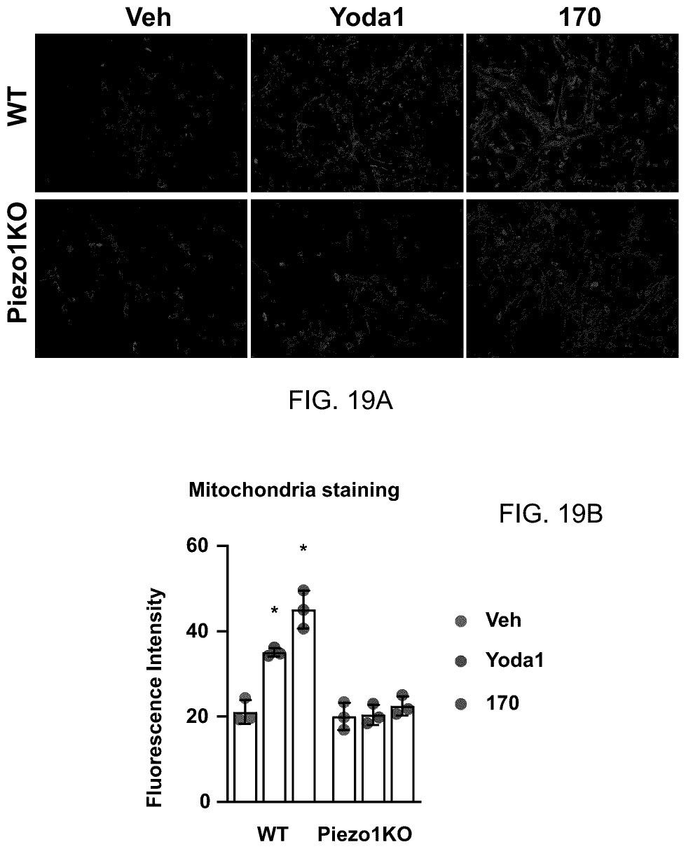

Next, we determined whether Yoda1 or YW-3-170 increase mitochondria number in primary calvaria cells. Wildtype and Piezo1 knockout calvarial cells were treated with 10 μM Yoda1 or YW-3-170 for 24 hours and then were stained for Mitochondria using MitoBright dye. We found that both Yoda1 and compound I increased mitochondria staining in wildtype calvaria cells (FIGS. 19A-19B). However, these increases were blunted in Piezo1 knockout cells. Importantly, YW-3-170 displayed more potent stimulation of mitochondria staining than Yoda1. The fluorescence intensity was quantified using the ImageJ software.

Taken together, these results suggest that activation of Piezo1 by Piezo1 agonists increases mitochondrial biogenesis and function in bone cells.

### REFERENCES

ABED, E., LABELLE, D., MARTINEAU, C., LOGHIN, A. & MOREAU, R. 2009. Expression of transient receptor potential (TRP) channels in human and murine osteoblast-like cells. Mol. Membr. Biol, 26, 146-158.
BIVI, N., CONDON, K. W., ALLEN, M. R., FARLOW, N., PASSERI, G., BRUN, L. R., RHEE, Y., BELLIDO, T. & PLOTKIN, L. I. 2012. Cell autonomous requirement of connexin 43 for osteocyte survival: consequences for endocortical resorption and periosteal bone formation. J. Bone Miner. Res, 27, 374-389.
BONEWALD, L. F. 2011. The amazing osteocyte. J. Bone Miner. Res, 26, 229-238.
CAHALAN, S. M., LUKACS, V., RANADE, S. S., CHIEN, S., BANDELL, M. & PATAPOUTIAN, A. 2015. Piezo1 links mechanical forces to red blood cell volume. Elife, 4.
CHOI, D., PARK, E., JUNG, E., CHA, B., LEE, S., YU, J., KIM, P. M., LEE, S., HONG, Y. J., KOH, C. J., CHO, C. W., WU, Y., LI, J. N., WONG, A. K., SHIN, L., KUMAR, S. R., BERMEJO-MORENO, I., SRINIVASAN, R. S., CHO, I. T. & HONG, Y. K. 2019. Piezo1 incorporates mechanical force signals into the genetic program that governs lymphatic valve development and maintenance. JCI. Insight, 4.
CONG, L., RAN, F. A., COX, D., LIN, S., BARRETTO, R., HABIB, N., HSU, P. D., WU, X., JIANG, W., MARRAFFINI, L. A. & ZHANG, F. 2013. Multiplex genome engineering using CRISPR/C as systems. Science, 339, 819-23.
COX, M. P., PETERSON, D. A. & BIGGS, P. J. 2010. SolexaQA: At-a-glance quality assessment of Illumina second-generation sequencing data. BMC. Bioinformatics, 11, 485.
DEMPSTER, D. W., COMPSTON, J. E., DREZNER, M. K., GLORIEUX, F. H., KANIS, J. A., MALLUCHE, H., MEUNIER, P. J., OTT, S. M., RECKER, R. R. & PARFITT, A. M. 2013. Standardized nomenclature, symbols, and units for bone histomorphometry: a 2012 update of the report of the ASBMR Histomorphometry Nomenclature Committee. J Bone Miner Res, 28, 2-17.
DUPONT, S., MORSUT, L., ARAGONA, M., ENZO, E., GIULITTI, S., CORDENONSI, M., ZANCONATO, F., LE, D. J., FORCATO, M., BICCIATO, S., ELVASSORE, N. & PICCOLO, S. 2011. Role of YAP/TAZ in mechanotransduction. Nature, 474, 179-183.
FOTIOU, E., MARTIN-ALMEDINA, S., SIMPSON, M. A., LIN, S., GORDON, K., BRICE, G., ATTON, G., JEFFERY, I., REES, D. C., MIGNOT, C., VOGT, J., HOMFRAY, T., SNYDER, M. P., ROCKSON, S. G., JEFFERY, S., MORTIMER, P. S., MANSOUR, S. & OSTERGAARD, P. 2015. Novel mutations in PIEZO1 cause an autosomal recessive generalized lymphatic dysplasia with non-immune hydrops fetalis. Nat. Commun, 6, 8085.
FU, Q., JILKA, R. L., MANOLAGAS, S. C. & O'BRIEN, C. A. 2002. Parathyroid hormone stimulates receptor activator of NFkappa B ligand and inhibits osteoprotegerin expression via protein kinase A activation of cAMP-response element-binding protein. J Biol Chem, 277, 48868-75.
GUDIPATY, S. A., LINDBLOM, J., LOFTUS, P. D., REDD, M. J., EDES, K., DAVEY, C. F., KRISHNEGOWDA, V. & ROSENBLATT, J. 2017. Mechanical stretch triggers rapid epithelial cell division through Piezo1. Nature, 543, 118-121.
HANSEN, C. G., MOROISHI, T. & GUAN, K. L. 2015. YAP and TAZ: a nexus for Hippo signaling and beyond. Trends Cell Biol, 25, 499-513.
HINTON, P. S., NIGH, P. & THYFAULT, J. 2015. Effectiveness of resistance training or jumping-exercise to increase bone mineral density in men with low bone mass: A 12-month randomized, clinical trial. Bone, 79, 203-212.
HOLGUIN, N., BRODT, M. D. & SILVA, M. J. 2016. Activation of Wnt Signaling by Mechanical Loading Is Impaired in the Bone of Old Mice. J. Bone Miner. Res, 31, 2215-2226.
HUNG, C. T., POLLACK, S. R., REILLY, T. M. & BRIGHTON, C. T. 1995. Real-time calcium response of cultured bone cells to fluid flow. Clin. Orthop. Relat Res, 256-269.
ILKAN, Z., WRIGHT, J. R., GOODALL, A. H., GIBBINS, J. M., JONES, C. I. & MAHAUT-SMITH, M. P. 2017. Evidence for shear-mediated Ca(2+) entry through mechanosensitive cation channels in human platelets and a megakaryocytic cell line. J. Biol. Chem, 292, 9204-9217.
JOENG, K. S., LEE, Y. C., LIM, J., CHEN, Y., JIANG, M. M., MUNIVEZ, E., AMBROSE, C. & LEE, B. H. 2017. Osteocyte-specific WNT1 regulates osteoblast function during bone homeostasis. J. Clin. Invest, 127, 2678-2688.
KATO, T., TERASHIMA, T., YAMASHITA, T., HATANAKA, Y., HONDA, A. & UMEMURA, Y. 2006. Effect of low-repetition jump training on bone mineral density in young women. J. Appl. Physiol (1985.), 100, 839-843.
KATO, Y., WINDLE, J. J., KOOP, B. A., MUNDY, G. R. & BONEWALD, L. F. 1997. Establishment of an osteocyte-like cell line, MLO-Y4. J Bone Miner Res, 12, 2014-23.
KELLY, N. H., SCHIMENTI, J. C., ROSS, F. P. & VAN DER MEULEN, M. C. 2016. Transcriptional profiling of cortical versus cancellous bone from mechanically-loaded murine tibiae reveals differential gene expression. Bone, 86, 22-29.
KLEIN-NULEND, J., BACABAC, R. G. & BAKKER, A. D. 2012. Mechanical loading and how it affects bone cells: the role of the osteocyte cytoskeleton in maintaining our skeleton. Eur. Cell Mater, 24, 278-291.
KLEIN-NULEND, J., BAKKER, A. D., BACABAC, R. G., VATSA, A. & WEINBAUM, S. 2013. Mechanosensation and transduction in osteocytes. Bone, 54, 182-190.
KONDO, H., NIFUJI, A., TAKEDA, S., EZURA, Y., RITTLING, S. R., DENHARDT, D. T., NAKASHIMA, K., KARSENTY, G. & NODA, M. 2005. Unloading induces osteoblastic cell suppression and osteoclastic cell activation to lead to bone loss via sympathetic nervous system. J. Biol. Chem, 280, 30192-30200.
LEE, K. L., GUEVARRA, M. D., NGUYEN, A. M., CHUA, M. C., WANG, Y. & JACOBS, C. R. 2015. The primary cilium functions as a mechanical and calcium signaling nexus. Cilia, 4, 7.
LEWIS, K. J., FRIKHA-BENAYED, D., LOUIE, J., STEPHEN, S., SPRAY, D. C., THI, M. M., SEREF-FERLENGEZ, Z., MAJESKA, R. J., WEINBAUM, S. & SCHAFFLER, M. B. 2017. Osteocyte calcium signals encode strain magnitude and loading frequency in vivo. Proc. Natl. Acad. Sci. U.S.A, 114, 11775-11780.
LI, H. & DURBIN, R. 2009. Fast and accurate short read alignment with Burrows-Wheeler transform. Bioinformatics, 25, 1754-1760.
LI, J., DUNCAN, R. L., BURR, D. B. & TURNER, C. H. 2002. L-type calcium channels mediate mechanically induced bone formation in vivo. J. Bone Miner. Res, 17, 1795-1800.
LI, J., HOU, B., TUMOVA, S., MURAKI, K., BRUNS, A., LUDLOW, M. J., SEDO, A., HYMAN, A. J., MCKEOWN, L., YOUNG, R. S., YULDASHEVA, N. Y., MAJEED, Y., WILSON, L. A., RODE, B., BAILEY, M. A., KIM, H. R., FU, Z., CARTER, D. A., BILTON, J., IMRIE, H., AJUH, P., DEAR, T. N., CUBBON, R. M., KEARNEY, M. T., PRASAD, R. K., EVANS, P. C., AINSCOUGH, J. F. & BEECH, D. J. 2014. Piezo1 integration of vascular architecture with physiological force. Nature, 515, 279-282.
LI, J., ZHAO, L., FERRIES, I. K., JIANG, L., DESTA, M. Z., YU, X., YANG, Z., DUNCAN, R. L. & TURNER, C. H. 2010. Skeletal phenotype of mice with a null mutation in Cav 1.3 L-type calcium channel. J. Musculoskelet. Neuronal. Interact, 10, 180-187.
LI, X., OMINSKY, M. S., NIU, Q. T., SUN, N., DAUGHERTY, B., D'AGOSTIN, D., KURAHARA, C., GAO, Y., CAO, J., GONG, J., ASUNCION, F., BARRERO, M., WARMINGTON, K., DWYER, D., STOLINA, M., MORONY, S., SAROSI, I., KOSTENUIK, P. J., LACEY, D. L., SIMONET, W. S., KE, H. Z. & PASZTY, C. 2008. Targeted deletion of the sclerostin gene in mice results in increased bone formation and bone strength. J. Bone Miner. Res, 23, 860-869.
LIM, J., BURCLAFF, J., HE, G., MILLS, J. C. & LONG, F. 2017. Unintended targeting of Dmp1-Cre reveals a critical role for Bmpr1a signaling in the gastrointestinal mesenchyme of adult mice. Bone Res, 5, 16049.
LITZENBERGER, J. B., KIM, J. B., TUMMALA, P. & JACOBS, C. R. 2010. Beta1 integrins mediate mechanosensitive signaling pathways in osteocytes. Calcif. Tissue Int, 86, 325-332.
LIVAK, K. J. & SCHMITTGEN, T. D. 2001. Analysis of relative gene expression data using real-time quantitative PCR and the 2(-Delta Delta C(T)) Method. methods, 25, 402-408.
LOVE, M. I., HUBER, W. & ANDERS, S. 2014. Moderated estimation of fold change and dispersion for RNA-seq data with DESeq2. Genome Biol, 15, 550.
LU, X. L., HUO, B., PARK, M. & GUO, X. E. 2012. Calcium response in osteocytic networks under steady and oscillatory fluid flow. Bone, 51, 466-473.
LUTHER, J., YORGAN, T. A., ROLVIEN, T., ULSAMER, L., KOEHNE, T., LIAO, N., KELLER, D., VOLLERSEN, N., TEUFEL, S., NEVEN, M., PETERS, S., SCHWEIZER, M., TRUMPP, A., ROSIGKEIT, S., BOCKAMP, E., MUNDLOS, S., KORNAK, U., OHEIM, R., AMLING, M., SCHINKE, T. & DAVID, J. P. 2018. Wnt1 is an Lrp5-independent bone-anabolic Wnt ligand. Sci. Transl. Med, 10.
MASUYAMA, R., VRIENS, J., VOETS, T., KARASHIMA, Y., OWSIANIK, G., VENNEKENS, R., LIEBEN, L., TORREKENS, S., MOERMANS, K., VANDEN BOSCH, A., BOUILLON, R., NILIUS, B. & CARMELIET, G. 2008. TRPV4-mediated calcium influx regulates terminal differentiation of osteoclasts. Cell Metab, 8, 257-265.
MEAKIN, L. B., GALEA, G. L., SUGIYAMA, T., LANYON, L. E. & PRICE, J. S. 2014. Age-related impairment of bones' adaptive response to loading in mice is associated with sex-related deficiencies in osteoblasts but no change in osteocytes. J. Bone Miner. Res, 29, 1859-1871.
MIYAMOTO, T., MOCHIZUKI, T., NAKAGOMI, H., KIRA, S., WATANABE, M., TAKAYAMA, Y., SUZUKI, Y., KOIZUMI, S., TAKEDA, M. & TOMINAGA, M. 2014. Functional role for Piezo1 in stretch-evoked Ca(2)(+) influx and ATP release in urothelial cell cultures. J. Biol. Chem, 289, 16565-16575.
MIZOGUCHI, F., MIZUNO, A., HAYATA, T., NAKASHIMA, K., HELLER, S., USHIDA, T., SOKABE, M., MIYASAKA, N., SUZUKI, M., EZURA, Y. & NODA, M. 2008. Transient receptor potential vanilloid 4 deficiency suppresses unloading-induced bone loss. J. Cell Physiol, 216, 47-53.
MORRIS, J. A., KEMP, J. P., YOULTEN, S. E., LAURENT, L., LOGAN, J. G., CHAI, R. C., VULPESCU, N. A., FORGETTA, V., KLEINMAN, A., MOHANTY, S. T., SERGIO, C. M., QUINN, J., NGUYEN-YAMAMOTO, L., LUCO, A. L., VIJAY, J., SIMON, M. M., PRAMATAROVA, A., MEDINA-GOMEZ, C., TRAJANOSKA, K., GHIRARDELLO, E. J., BUTTERFIELD, N. C., CURRY, K. F., LEITCH, V. D., SPARKES, P. C., ADOUM, A. T., MANNAN, N. S., KOMLA-EBRI, D. S. K., POLLARD, A. S., DEWHURST, H. F., HASSALL, T. A. D., BELTEJAR, M. G., ADAMS, D. J., VAILLANCOURT, S. M., KAPTOGE, S., BALDOCK, P., COOPER, C., REEVE, J., NTZANI, E. E., EVANGELOU, E., OHLSSON, C., KARASIK, D., RIVADENEIRA, F., KIEL, D. P., TOBIAS, J. H., GREGSON, C. L., HARVEY, N. C., GRUNDBERG, E., GOLTZMAN, D., ADAMS, D. J., LELLIOTT, C. J., HINDS, D. A., ACKERT-BICKNELL, C. L., HSU, Y. H., MAURANO, M. T., CROUCHER, P. I., WILLIAMS, G. R., BASSETT, J. H. D., EVANS, D. M. & RICHARDS, J. B. 2019. An atlas of genetic influences on osteoporosis in humans and mice. Nat. Genet, 51, 258-266.

MURTHY, S. E., DUBIN, A. E. & PATAPOUTIAN, A. 2017. Piezos thrive under pressure: mechanically activated ion channels in health and disease. Nat. Rev. Mol. Cell Biol, 18, 771-783.
NAKAMURA, H., AOKI, K., MASUDA, W., ALLES, N., NAGANO, K., FUKUSHIMA, H., OSAWA, K., YASUDA, H., NAKAMURA, I., MIKUNI-TAKAGAKI, Y., OHYA, K., MAKI, K. & JIMI, E. 2013. Disruption of NF-kappaB1 prevents bone loss caused by mechanical unloading. J. Bone Miner. Res, 28, 1457-1467.
NGUYEN, A. M. & JACOB S, C. R. 2013. Emerging role of primary cilia as mechanosensors in osteocytes. Bone, 54, 196-204.
NOOKAEW, I., PAPINI, M., PORNPUTTAPONG, N., SCALCINATI, G., FAGERBERG, L., UHLEN, M. & NIELSEN, J. 2012. A comprehensive comparison of RNA-Seq-based transcriptome analysis from reads to differential gene expression and cross-comparison with microarrays: a case study in Saccharomyces cerevisiae. Nucleic Acids Res, 40, 10084-10097.
O'BRIEN, C. A., GUBRIJ, I., LIN, S. C., SAYLORS, R. L. & MANOLAGAS, S. C. 1999. STAT3 activation in stromal/osteoblastic cells is required for induction of the receptor activator of NF-kappaB ligand and stimulation of osteoclastogenesis by gp130-utilizing cytokines or interleukin-1 but not 1,25-dihydroxyvitamin D3 or parathyroid hormone. J Biol Chem, 274, 19301-8.
OZCIVICI, E., LUU, Y. K., ADLER, B., QIN, Y. X., RUBIN, J., JUDEX, S. & RUBIN, C. T. 2010. Mechanical signals as anabolic agents in bone. Nat. Rev. Rheumatol, 6, 50-59.
PATHAK, M. M., NOURSE, J. L., TRAN, T., HWE, J., ARULMOLI, J., LE, D. T., BERNARDIS, E., FLANAGAN, L. A. & TOMBOLA, F. 2014. Stretch-activated ion channel Piezo1 directs lineage choice in human neural stem cells. Proc. Natl. Acad. Sci. U.S.A, 111, 16148-16153.
RODE, B., SHI, J., ENDESH, N., DRINKHILL, M. J., WEBSTER, P. J., LOTTEAU, S. J., BAILEY, M. A., YULDASHEVA, N. Y., LUDLOW, M. J., CUBBON, R. M., LI, J., FUTERS, T. S., MORLEY, L., GAUNT, H. J., MARSZALEK, K., VISWAMBHARAN, H., CUTHBERTSON, K., BAXTER, P. D., FOSTER, R., SUKUMAR, P., WEIGHTMAN, A., CALAGHAN, S. C., WHEATCROFT, S. B., KEARNEY, M. T. & BEECH, D. J. 2017. Piezo1 channels sense whole body physical activity to reset cardiovascular homeostasis and enhance performance. Nat. Commun, 8, 350.
RUBIN, J., RUBIN, C. & JACOBS, C. R. 2006. Molecular pathways mediating mechanical signaling in bone. Gene, 367, 1-16.
SHAO, Y., ALICKNAVITCH, M. & FARACH-CARSON, M. C. 2005. Expression of voltage sensitive calcium channel (VSCC) L-type Cav1.2 (alpha1C) and T-type Cav3.2 (alpha1H) subunits during mouse bone development. Dev. Dyn, 234, 54-62.
SUN, D., BRODT, M. D., ZANNIT, H. M., HOLGUIN, N. & SILVA, M. J. 2018. Evaluation of loading parameters for murine axial tibial loading: Stimulating cortical bone formation while reducing loading duration. J. Orthop. Res, 36, 682-691.
SUN, W., CHI, S., LI, Y., LING, S., TAN, Y., XU, Y., JIANG, F., LI, J., LIU, C., ZHONG, G., CAO, D., JIN, X., ZHAO, D., GAO, X., LIU, Z., XIAO, B. & LI, Y. 2019. The mechanosensitive Piezo1 channel is required for bone formation. Elife, 8.
SUZUKI, T., NOTOMI, T., MIYAJIMA, D., MIZOGUCHI, F., HAYATA, T., NAKAMOTO, T., HANYU, R., KAMOLRATANAKUL, P., MIZUNO, A., SUZUKI, M., EZURA, Y., IZUMI, Y. & NODA, M. 2013. Osteoblastic differentiation enhances expression of TRPV4 that is required for calcium oscillation induced by mechanical force. Bone, 54, 172-178.
SYEDA, R., FLORENDO, M. N., COX, C. D., KEFAUVER, J. M., SANTOS, J. S., MARTINAC, B. & PATAPOUTIAN, A. 2016. Piezo1 Channels Are Inherently Mechanosensitive. Cell Rep, 17, 1739-1746.
SYEDA, R., XU, J., DUBIN, A. E., COSTE, B., MATHUR, J., HUYNH, T., MATZEN, J., LAO, J., TULLY, D. C., ENGELS, I. H., PETRASSI, H. M., SCHUMACHER, A. M., MONTAL, M., BANDELL, M. & PATAPOUTIAN, A. 2015. Chemical activation of the mechanotransduction channel Piezo1. Elife, 4.
TURNER, C. H., WARDEN, S. J., BELLIDO, T., PLOTKIN, L. I., KUMAR, N., JASIUK, I., DANZIG, J. & ROBLING, A. G. 2009. Mechanobiology of the skeleton. Sci. Signal, 2, t3.
VAN DER EERDEN, B. C., OEI, L., ROSCHGER, P., FRATZL-ZELMAN, N., HOENDEROP, J. G., VAN SCHOOR, N. M., PETTERSSON-KYMMER, U., SCHREUDERS-KOEDAM, M., UITTERLINDEN, A. G., HOFMAN, A., SUZUKI, M., KLAUSHOFER, K., OHLSSON, C., LIPS, P. J., RIVADENEIRA, F., BINDELS, R. J. & VAN LEEUWEN, J. P. 2013. TRPV4 deficiency causes sexual dimorphism in bone metabolism and osteoporotic fracture risk. Bone, 57, 443-454.
VAREMO, L., NIELSEN, J. & NOOKAEW, I. 2013. Enriching the gene set analysis of genome-wide data by incorporating directionality of gene expression and combining statistical hypotheses and methods. Nucleic Acids Res, 41, 4378-4391.
VLACHOPOULOS, D., BARKER, A. R., UBAGO-GUISADO, E., WILLIAMS, C. A. & GRACIA-MARCO, L. 2018. The effect of a high-impact jumping intervention on bone mass, bone stiffness and fitness parameters in adolescent athletes. Arch. Osteoporos, 13, 128.
WADHWA, S., GODWIN, S. L., PETERSON, D. R., EPSTEIN, M. A., RAISZ, L. G. & PILBEAM, C. C. 2002. Fluid flow induction of cyclo-oxygenase 2 gene expression in osteoblasts is dependent on an extracellular signal-regulated kinase signaling pathway. J. Bone Miner. Res, 17, 266-274.
WANG, K. C., YEH, Y. T., NGUYEN, P., LIMQUECO, E., LOPEZ, J., THOROSSIAN, S., GUAN, K. L., LI, Y. J. & CHIEN, S. 2016. Flow-dependent YAP/TAZ activities regulate endothelial phenotypes and atherosclerosis. Proc. Natl. Acad. Sci. U.S.A, 113, 11525-11530.
XIN, M., KIM, Y., SUTHERLAND, L. B., MURAKAMI, M., QI, X., MCANALLY, J., PORRELLO, E. R., MAHMOUD, A. I., TAN, W., SHELTON, J. M., RICHARDSON, J. A., SADEK, H. A., BASSEL-DUBY, R. & OLSON, E. N. 2013. Hippo pathway effector Yap1 promotes cardiac regeneration. Proc. Natl. Acad. Sci. U.S.A, 110, 13839-13844.
XIONG, J., ALMEIDA, M. & O'BRIEN, C. A. 2018. The YAP/TAZ transcriptional co-activators have opposing effects at different stages of osteoblast differentiation. Bone, 112, 1-9.
XIONG, J., ONAL, M., JILKA, R. L., WEINSTEIN, R. S., MANOLAGAS, S. C. & O'BRIEN, C. A. 2011. Matrix-embedded cells control osteoclast formation. Nat. Med, 17, 1235-1241.
XIONG, J., PIEMONTESE, M., ONAL, M., CAMPBELL, J., GOELLNER, J. J., DUSEVICH, V., BONEWALD, L., MANOLAGAS, S. C. & O'BRIEN, C. A. 2015. Osteocytes, not Osteoblasts or Lining Cells, are the Main Source of the RANKL Required for Osteoclast Formation in Remodeling Bone. PLoS. One, 10, e0138189.
ZHANG, T., CHI, S., JIANG, F., ZHAO, Q. & XIAO, B. 2017. A protein interaction mechanism for suppressing the mechanosensitive Piezo channels. Nat Commun, 8, 1797.
ZHAO, N., NOCITI, F. H., JR., DUAN, P., PRIDEAUX, M., ZHAO, H., FOSTER, B. L., SOMERMAN, M. J. & BONEWALD, L. F. 2016. Isolation and Functional Analysis of an Immortalized Murine Cementocyte Cell Line, IDG-CM6. J. Bone Miner. Res, 31, 430-442.

1. A compound having formula (I) or a pharmaceutically acceptable salt thereof 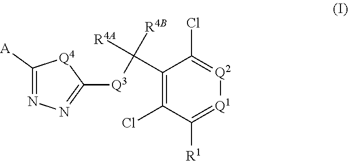
wherein
(a) Q¹ and Q² are:
(i) CR² and CR³, respectively, and each of R¹, R², and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl;
(ii) CR² and N, respectively, and each of R¹ and R² are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl; or
(iii) N and CR³, respectively, and each of R¹ and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl;
(b) R^{4A} and R^{4B} are independently selected from hydrogen or a C₁-C₆ alkyl or R^{4A} and R^{4B} are together selected from an oxo group;
(c) Q³ is selected from S, O, or NH;
(d) Q⁴ is selected from S, O, or NR⁷, wherein R⁷ is selected from hydrogen, a C₁-C₆ alkyl, or an aryl; and
(e) A is a substituted or unsubstituted heteroaryl having at least 1 N in the heteroaryl ring,
wherein each of R¹, R², and R³ are not hydrogen if R^{4A} and R^{4B} are each hydrogen, Q³ and Q⁴ are each S, and A is pyrazin-2-yl.

2. (canceled)

3. (canceled)

4. The compound of any claim 1, wherein R^{4A} and R^{4B} are each hydrogen, Q³ is S, Q⁴ is S, A is a substituted or unsubstituted pyrazinyl, or any combination thereof.

5. (canceled)

6. (canceled)

7. (canceled)

8. (canceled)

9. (canceled)

10. The compound of claim 4, wherein the compound is

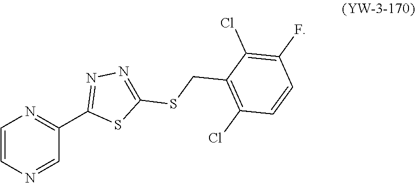 [SMILES: Fc1ccc(Cl)c(CSc2nnc(-c3cnccn3)s2)c1Cl]

11. The compound of claim 1, wherein Q¹ and Q² are CR² and N, respectively, and each of R¹ and R² are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl.

12. The compound of claim 11, wherein R^{4A} and R^{4B} are each hydrogen, Q³ is S, Q⁴ is S, A is a substituted or unsubstituted pyrazinyl, or any combination thereof.

13. (canceled)

14. (canceled)

15. (canceled)

16. (canceled)

17. (canceled)

18. The compound of claim 12, wherein the compound is

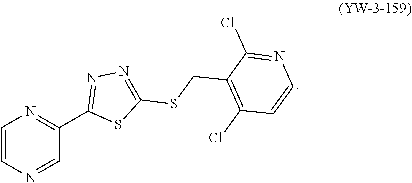 [SMILES: Clc1ccnc(Cl)c1CSc1nnc(-c2cnccn2)s1]

19. The compound of claim 1, wherein Q¹ and Q² are N and CR³, respectively, and each of R¹ and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl.

20. The compound of claim 19, wherein R^{4A} and R^{4B} are each hydrogen, Q³ is S, Q⁴ is S, A is a substituted or unsubstituted pyrazinyl, or any combination thereof.

21. (canceled)

22. (canceled)

23. (canceled)

24. (canceled)

25. (canceled)

26. The compound of claim 20, wherein the compound is YW-3-168-1

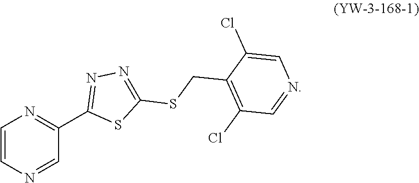 [SMILES: Clc1cncc(Cl)c1CSc1nnc(-c2cnccn2)s1]

27. The compound of claim 1, wherein the compound is selected from

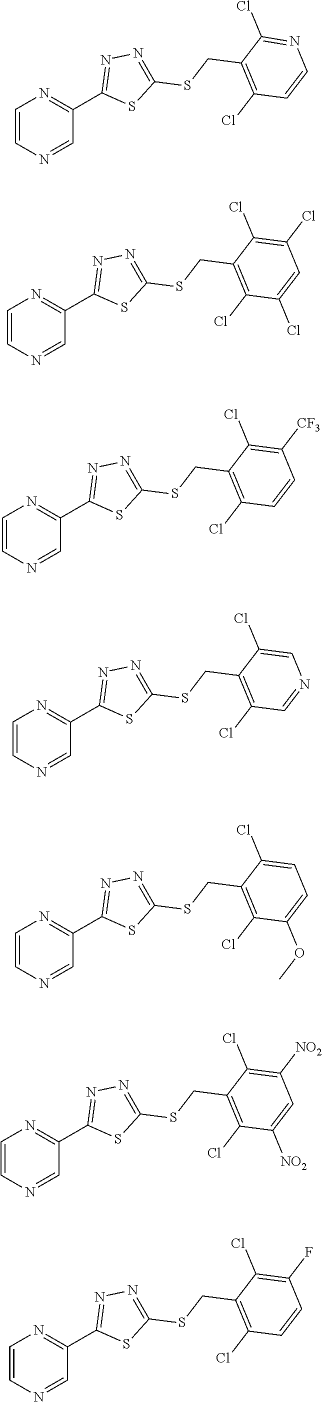

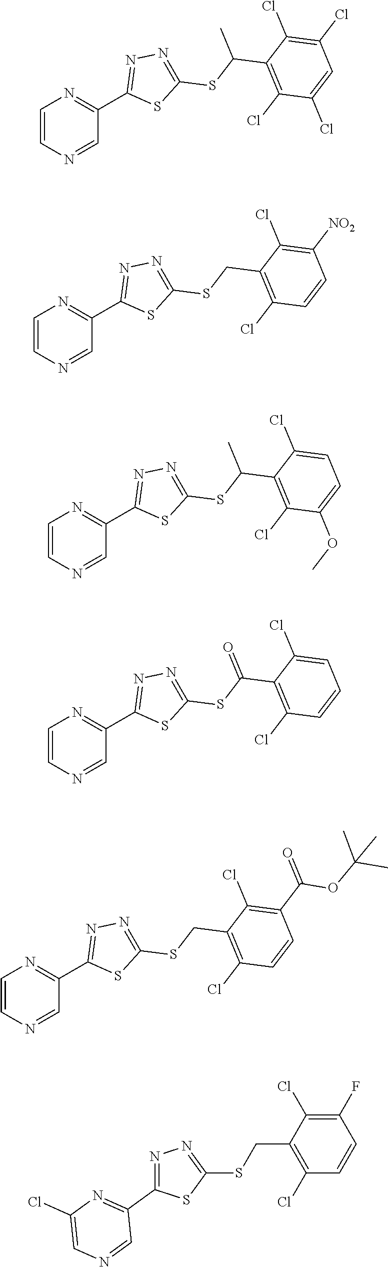 [SMILES: CC(Sc1nnc(-c2cnccn2)s1)c1c(Cl)c(Cl)cc(Cl)c1Cl ; O=[N+]([O-])c1ccc(Cl)c(CSc2nnc(-c3cnccn3)s2)c1Cl ; COc1ccc(Cl)c(C(C)Sc2nnc(-c3cnccn3)s2)c1Cl ; O=C(Sc1nnc(-c2cnccn2)s1)c1c(Cl)cccc1Cl ; CC(C)(C)OC(=O)c1ccc(Cl)c(CSc2nnc(-c3cnccn3)s2)c1Cl ; Fc1ccc(Cl)c(CSc2nnc(-c3cncc(Cl)n3)s2)c1Cl]

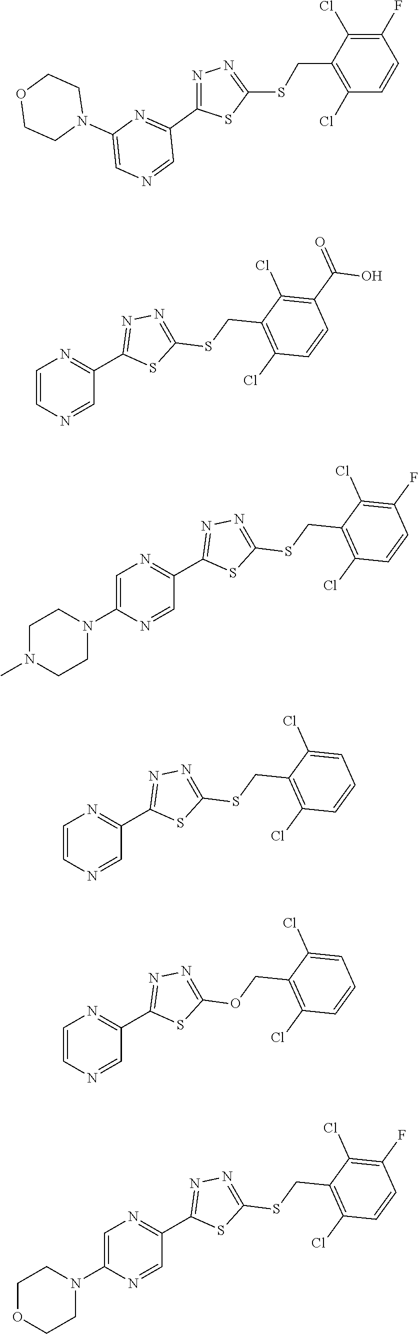 [SMILES: Fc1ccc(Cl)c(CSc2nnc(-c3cncc(N4CCOCC4)n3)s2)c1Cl ; O=C(O)c1ccc(Cl)c(CSc2nnc(-c3cnccn3)s2)c1Cl ; CN1CCN(c2cnc(-c3nnc(SCc4c(Cl)ccc(F)c4Cl)s3)cn2)CC1 ; Clc1cccc(Cl)c1CSc1nnc(-c2cnccn2)s1 ; Clc1cccc(Cl)c1COc1nnc(-c2cnccn2)s1 ; Fc1ccc(Cl)c(CSc2nnc(-c3cnc(N4CCOCC4)cn3)s2)c1Cl]

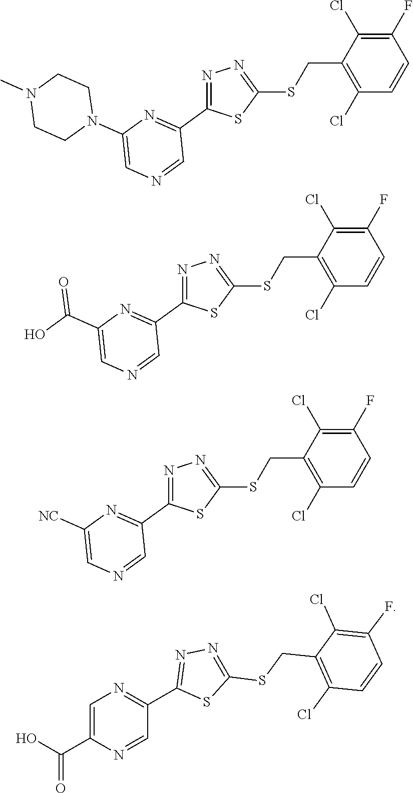 [SMILES: CN1CCN(c2cncc(-c3nnc(SCc4c(Cl)ccc(F)c4Cl)s3)n2)CC1 ; O=C(O)c1cncc(-c2nnc(SCc3c(Cl)ccc(F)c3Cl)s2)n1 ; N#Cc1cncc(-c2nnc(SCc3c(Cl)ccc(F)c3Cl)s2)n1 ; O=C(O)c1cnc(-c2nnc(SCc3c(Cl)ccc(F)c3Cl)s2)cn1]

28. A pharmaceutical composition comprising a therapeutically effective amount of the compound as in claim 1 and a pharmaceutically acceptable carrier, diluent, or excipient.

29. A method for stimulating tissue anabolism in a subject, the method comprising administering to the subject an effective amount of a Piezo1 agonist or a pharmaceutical composition comprising the effective amount of the Piezo1 agonist, wherein the tissue comprises cells expressing Piezo1.

30. The method of claim 29, wherein the tissue is bone tissue and/or the cells expressing Piezo1 are osteoblasts or osteocytes.

31. The method of claim 30, wherein the subject is in need of a treatment to prevent or reverse loss of bone mass, bone mineral content, or bone strength.

32. The method of claim 30, wherein the subject displays or is predisposed to developing a bone-depletive disorder.

33. (canceled)

34. The method of claim 30, wherein the subject is in need of a bone healing treatment.

35. The method of claim 30, wherein the subject has a bone fracture.

36. The method of claim 29, wherein the Piezo1 agonist is compound having formula (I) or a pharmaceutically acceptable salt thereof 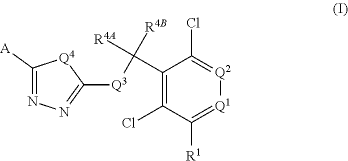
wherein
(a) Q¹ and Q² are:
(i) CR² and CR³, respectively, and each of R¹, R², and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl;
(ii) CR² and N, respectively, and each of R¹ and R² are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl; or
(iii) N and CR³, respectively, and each of R¹ and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl;
(b) R^{4A} and R^{4B} are independently selected from hydrogen or a C₁-C₆ alkyl or R^{4A} and R^{4B} are together selected from an oxo group;
(c) Q³ is selected from S, O, or NH;
(d) Q⁴ is selected from S, O, or NR⁷, wherein R⁷ is selected from hydrogen, a C₁-C₆ alkyl, or an aryl; and
(e) A is a substituted or unsubstituted heteroaryl having at least 1 N in the heteroaryl ring.

37. (canceled)

38. A method for chemically mimicking mechanical stimulation of a cell expressing Piezo1, the method comprising contacting the cell expressing Piezo1 with an effective amount of a Piezo1 agonist.

39. (canceled)

40. (canceled)

41. (canceled)

42. The method of claim 38, wherein the Piezo1 agonist is compound having formula (I) or a pharmaceutically acceptable salt thereof 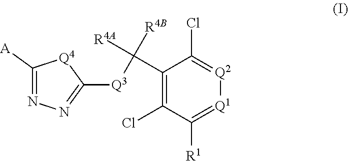
wherein
(a) Q¹ and Q² are:
(i) CR² and CR³, respectively, and each of R¹, R², and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl;
(ii) CR² and N, respectively, and each of R¹ and R² are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl; or
(iii) N and CR³, respectively, and each of R¹ and R³ are independently selected from hydrogen, a halogen, NO₂, a C₁-C₆ haloalkyl, a C(O)OR⁶, or a C₁-C₆ alkoxy, wherein R⁶ is independently hydrogen or a C₁-C₆ alkyl;
(b) R^{4A} and R^{4B} are independently selected from hydrogen or a C₁-C₆ alkyl or R^{4A} and R^{4B} are together selected from an oxo group;
(c) Q³ is selected from S, O, or NH;
(d) Q⁴ is selected from S, O, or NR', wherein R⁷ is selected from hydrogen, a C₁-C₆ alkyl, or an aryl; and
(e) A is a substituted or unsubstituted heteroaryl having at least 1 N in the heteroaryl ring.

43. (canceled)


SEQUENCE LISTING (6 sequences)

SEQ ID NO: 1 - DNA, 58 bp, Artificial Sequence
  Synthetic- Piezo1 shRNA
ccggtcggcgcttgctagaacttcactcgagtgaagttctagcaagcgccgatttttg

SEQ ID NO: 2 - DNA, 58 bp, Artificial Sequence
  Synthetic- Piezo1 shRNA
aattcaaaaatcggcgcttgctagaacttcactcgagtgaagttctagcaagcgccga

SEQ ID NO: 3 - DNA, 20 bp, Artificial Sequence
  Synthetic- Piezo1 sgRNA
ggttattcctgtgaggcccg

SEQ ID NO: 4 - DNA, 20 bp, Artificial Sequence
  Synthetic- Piezo1 sgRNA
ttaggattcggctcacagag

SEQ ID NO: 5 - DNA, 21 bp, Artificial Sequence
  Synthetic- screening primer
gctgtcagggtaagcagtatc

SEQ ID NO: 6 - DNA, 21 bp, Artificial Sequence
  Synthetic- screening primer
ggaatatgaggacagcagtcc